(19) United States
(12) Patent Application Publication
(10) Pub. No.: US 2013/0012491 A1
(43) Pub. Date: Jan. 10, 2013

(54) PYRIMIDINE DERIVATIVES FOR USE AS SPHINGOSINE 1-PHOSPHATE 1 (S1P1) RECEPTOR AGONISTS

(75) Inventors: Xichen Lin, Shanghai (CN); Feng Ren, Shanghai (CN); Haibo Zhang, Shanghai (CN)

(73) Assignee: GLAXO GROUP LIMITED, Greenford, Middlesex (GB)

(21) Appl. No.: 13/635,499

(22) PCT Filed: Mar. 16, 2011
(86) PCT No.: PCT/CN2011/000432

§ 371 Date: Sep. 17, 2012

(30) Foreign Application Priority Data
Mar. 17, 2010 (CN) PCT/CN2010/071101

(51) Int. Cl. C07D 239/26; C07D 401/04; A61P 25/00; A61K 31/506; C07D 401/10; A61K 31/505; C07D 403/10

(52) U.S. Cl. 514/210.18; 544/335; 514/256; 544/333

(57) ABSTRACT

Disclosed are pyrimidine derivatives for use as a sphingosine 1-phosphate 1 (S1P1) receptor agonists, processes for their preparation, pharmaceutical compositions containing them and their use in the treatment of conditions or diseases mediated by S1P1 receptors, particularly multiple sclerosis.

The present invention relates to novel pyrimidine compounds having pharmacological activity, processes for their preparation, pharmaceutical compositions containing them and their use in the treatment of various disorders.

Sphingosine 1-phosphate (SIP) is a bioactive lipid mediator formed by the phosphorylation of sphingosine by sphingosine kinases and is found in high levels in the blood. It is produced and secreted by a number of cell types, including those of hematopoietic origin such as platelets and mast cells (Okamoto et al 1998 J Biol Chem 273(42):27104; Sanchez and Hla 2004, J Cell Biochem 92:913). It has a wide range of biological actions, including regulation of cell proliferation, differentiation, motility, vascularisation, and activation of inflammatory cells and platelets (Pyne and Pyne 2000, Biochem J. 349: 385). Five subtypes of S1P responsive receptor have been described, S1P1 (Edg-1), S1P2 (Edg-5), S1P3 (Edg-3), S1P4 (Edg-6), and S1P5 (Edg-8), forming part of the G-protein coupled endothelial differentiation gene family of receptors (Chun et at 2002 Pharmacological Reviews 54:265, Sanchez and Hla 2004 J Cellular Biochemistry, 92:913). These 5 receptors show differential mRNA expression, with S1P1-3 being widely expressed, S1P4 expressed on lymphoid and hematopoietic tissues and S1P5 primarily in brain and to a lower degree in spleen. They signal via different subsets of G proteins to promote a variety of biological responses (Kluk and Hla 2002 Biochem et Biophysida Acta 1582:72, Sanchez and Hla 2004, J Cellular Biochem 92:913).

Proposed roles for the S1P1 receptor include lymphocyte trafficking, cytokine induction/suppression and effects on endothelial cells (Rosen and Goetzl 2005 Nat Rev Immunol. 5:560). Agonists of the S1P1 receptor have been used in a number of autoimmune and transplantation animal models, including Experimental Autoimmune Encephalomelitis (EAE) models of MS, to reduce the severity of the induced disease (Brinkman et at 2003 JBC 277:21453; Fujino et al 2003 J Pharmacol Exp Ther 305:70; Webb et al 2004 J Neuroimmunol 153:108; Rausch et al 2004 J Magn Reson Imaging 20:16). This activity is reported to be mediated by the effect of S1P1 agonists on lymphocyte circulation through the lymph system. Treatment with S1P1 agonists results in the sequestration of lymphocytes within secondary lymphoid organs such as the lymph nodes, inducing a reversible peripheral lymphopoenia in animal models (Chiba et al 1998, J Immunology 160:5037, Forrest et al 2004 J Pharmacol Exp Ther 309:758; Sanna et at 2004 JBC 279:13839). Published data on agonists suggests that compound treatment induces loss of the S1P1 receptor from the cell surface via internalisation (Grater and Goetzl 2004 FASEB J 18:551; Matloubian et at 2004 Nature 427:355; Jo et at 2005 Chem Biol 12:703) and it is this reduction of S1P1 receptor on immune cells which contributes to the reduction of movement of T cells from the lymph nodes back into the blood stream.

S1P1 gene deletion causes embryonic lethality. Experiments to examine the role of the S1P1 receptor in lymphocyte migration and trafficking have included the adoptive transfer of labelled S1P1 deficient T cells into irradiated wild type mice. These cells showed a reduced egress from secondary lymphoid organs (Matloubian et al 2004 Nature 427:355).

S1P1 has also been ascribed a role in endothelial cell junction modulation (Allende et at 2003 102:3665, Blood Singelton et al 2005 FASEB J 19:1646). With respect to this endothelial action, S1P1 agonists have been reported to have an effect on isolated lymph nodes which may be contributing to a role in modulating immune disorders. S1P1 agonists caused a closing of the endothelial stromal ‘gates’ of lymphatic sinuses which drain the lymph nodes and prevent lymphocyte egress (Wei wt at 2005, Nat. Immunology 6:1228).

The immunosuppressive compound FTY720 (JP11080026-A) has been shown to reduce circulating lymphocytes in animals and man, have disease modulating activity in animal models of immune disorders and reduce remission rates in relapsing remitting Multiple Sclerosis (Brinkman et at 2002 JBC 277:21453, Mandate et at 2002 Science 296:346, Fujino et at 2003 J Pharmacology and Experimental Therapeutics 305:45658, Brinkman et at 2004 American J Transplantation 4:1019, Webb et at 2004 J Neuroimmunology 153:108, Morris et at 2005 Eur J Immunol 35:3570, Chiba 2005 Pharmacology and Therapeutics 108:308, Kahan et at 2003, Transplantation 76:1079, Kappos et at 2006 New Eng J Medicine 335:1124). This compound is a prodrug that is phosphorylated in vivo by sphingosine kinases to give a molecule that has agonist activity at the S1P1, S1P3, S1P4 and S1P5 receptors. Clinical studies have demonstrated that treatment with FTY720 results in bradycardia in the first 24 hours of treatment (Kappos et at 2006 New Eng J Medicine 335:1124). The bradycardia is thought to be due to agonism at the S1P3 receptor, based on a number of cell based and animal experiments. These include the use of S1P3 knock-out animals which, unlike wild type mice, do not demonstrate bradycardia following FTY720 administration and the use of S1P1 selective compounds. (Hale et al 2004 Bioorganic & Medicinal Chemistry Letters 14:3501, Senna et at 2004 JBC 279:13839, Koyrakh et al 2005 American J Transplantation 5:529)

Hence, there is a need for S1P1 receptor agonist compounds with selectivity over S1P3 which might be expected to show a reduced tendency to induce bradycardia.

A structurally novel class of compounds has now been found which provides agonists of the S1P1 receptor.

The present invention therefore provides compounds of formula (I) or a salt thereof:

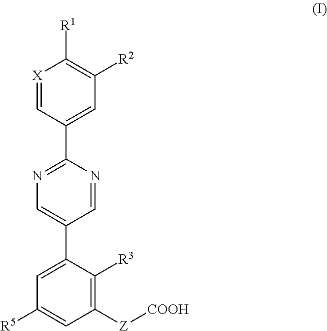

wherein

### X is CH or N;

R¹ is C₁₋₆alkoxy or C₁₋₆alkyl;
R² is cyano, CF₃, halogen C₁₋₄alkoxy or CH₂OCH₃;
R³ is C₁₋₆alkoxy or C₁₋₆alkyl;
Z is C₁₋₅alkyl, C₀₋₃alkylOC₁₋₅alkyl or C₀₋₃alkylNR⁴C₀₋₅alkyl, each of which may be optionally substituted by one to three C₁₋₃alkyl groups;
R⁴ is hydrogen, C₁₋₃alkyl or together with the nitrogen atom to which it is attached forms azetidine, pyrrolidine or piperidine; and
R⁵ is hydrogen, halogen or C₁₋₃alkyl.

In one embodiment X is CH. In another embodiment X is N.

In one embodiment R¹ is C₁₋₆alkoxy or C₁₋₆alkyl. In a further embodiment R¹ is isopropoxy or isobutyl. In another embodiment R¹ is isopropoxy.

In one embodiment R² is cyano, CF₃ or halogen. In another embodiment R² is cyano, CF₃ or chloro. In a further embodiment R² is cyano or chloro.

In one embodiment R³ is C₁₋₆alkoxy or C₁₋₆alkyl. In another embodiment R³ is methoxy or ethyl. In a further embodiment R³ is ethyl.

In one embodiment Z is C₁₋₃alkyl, C₀alkylOC₃alkyl or C₁₋₆alkylNR⁴C₀₋₂alkyl each of which may be optionally substituted by C₁₋₃alkyl. In one embodiment Z is C₁₋₃alkyl or C₁alkylNR⁴C_{0.2}alkyl, each of which may be optionally substituted by C₁₋₃alkyl. In another embodiment Z is (CH₂)₃. In another embodiment Z is C₁alkylNR⁴C_{0.2}alkyl, which may be optionally substituted by C₁₋₃alkyl.

In one embodiment R⁴ is hydrogen, C₁₋₃alkyl or together with the nitrogen atom to which it is attached forms azetidine, pyrollidine or piperidine. In another embodiment R⁴ is hydrogen, methy, ethyl or together with the nitrogen atom to which it is attached forms azetidine, pyrollidine or piperidine.

In one embodiment R⁵ is hydrogen or fluoro.

In one embodiment

### X is CH or N;

R¹ is C₁₋₆alkoxy or C₁₋₆alkyl;
R² is cyano, CF₃ or halogen;
R³ is C₁₋₆alkoxy or C₁₋₆alkyl;
Z is C₁₋₃alkyl, C₀alkylOC₃alkyl or C₁alkylNR⁴C₀₋₂alkyl, each of which may be optionally substituted by C₁₋₃alkyl;
R⁴ is hydrogen, C₁₋₃alkyl or together with the nitrogen atom to which it is attached forms azetidine, pyrollidine or piperidine; and
R⁵ is hydrogen or halogen.

In one embodiment:

### X is CH or N;

R¹ is isopropoxy;
R² is cyano, CF₃ or chloro;
R³ is methoxy or ethyl;
Z is (CH₂)₃ or together with the nitrogen atom to which it is attached forms azetidine, pyrollidine or piperidine and
R⁵ is hydrogen or fluoro.

In one embodiment:

### X is CH or N;

R¹ is isopropoxy;
R² is cyano, CF₃ or chloro;
R³ is methoxy or ethyl;
Z is C₁alkylNR⁴C₀₋₂alkyl, which may be optionally substituted by C₁₋₃alkyl;
R⁴ is hydrogen, methy, ethyl or together with the nitrogen atom to which it is attached forms azetidine, pyrollidine or piperidine; and
R⁵ is hydrogen.

In a further embodiment:

### X is CH or N;

R¹ is isopropoxy;
R² is cyano, CF₃ or chloro;
R³ is methoxy or ethyl;
Z is C₁alkylNR⁴C_{0.2}alkyl, which may be optionally substituted by cyclopropyl;
R⁴ is hydrogen, methy, ethyl or together with the nitrogen atom to which it is attached forms azetidine, pyrollidine or piperidine; and
R⁵ is hydrogen.

The term “alkyl” as a group or part of a group e.g. alkoxy refers to a straight or branched alkyl group in all isomeric forms. The term “C₍₁₋₆₎ alkyl” refers to an alkyl group, as defined above, containing at least 1, and at most 6 carbon atoms Examples of such alkyl groups include methyl, ethyl, propyl, iso-propyl, n-butyl, iso-butyl, sec-butyl, or tert-butyl. Examples of such alkoxy groups include methoxy, ethoxy, propoxy, iso-propoxy, butoxy, iso-butoxy, sec-butoxy and tert-butoxy.

In a further aspect, this invention provides processes for preparation of a compound of formula (I), where R₁ and R₂, and X in scheme I are as defined for formula (I).

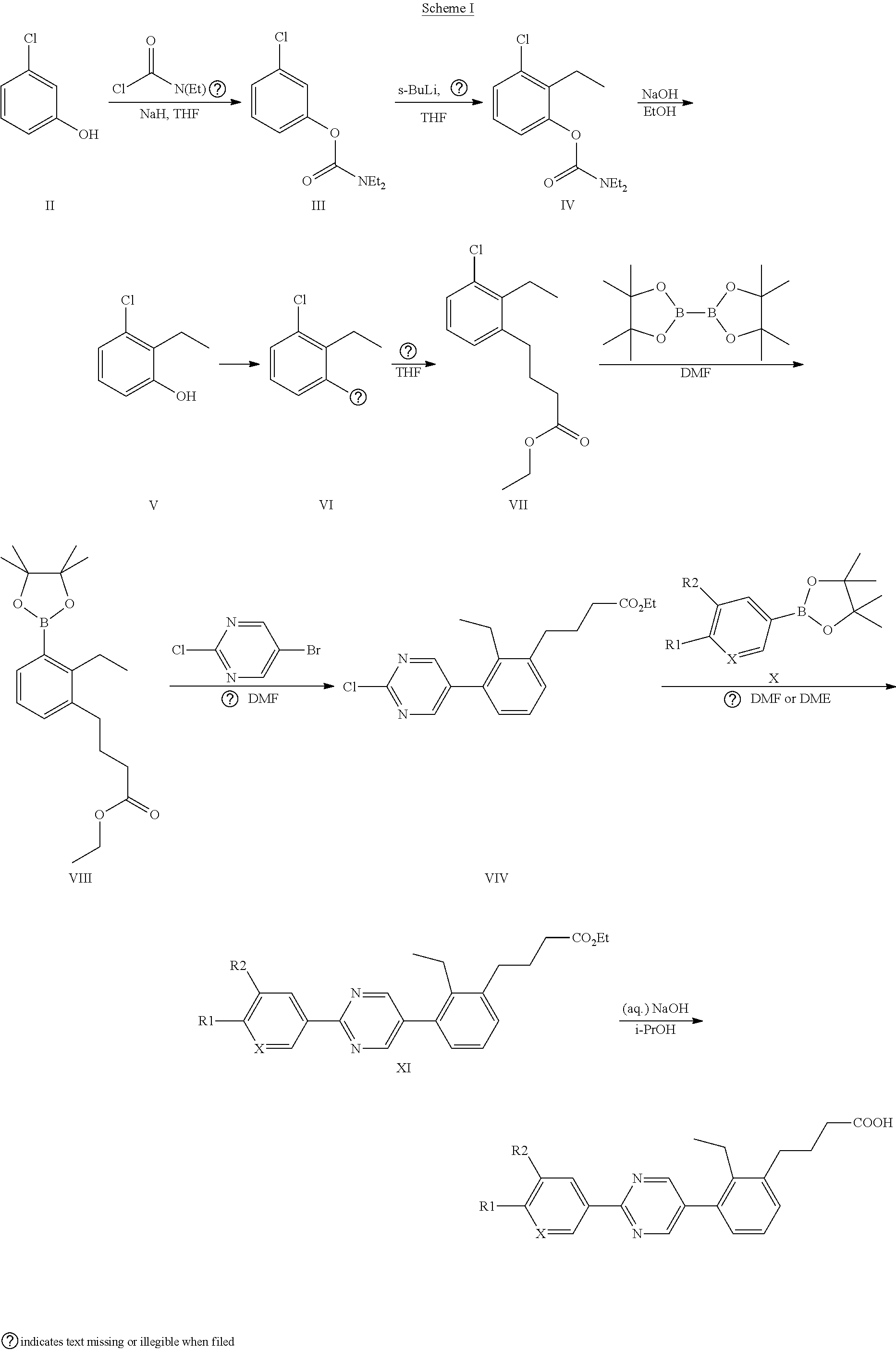

The first step of the process (II to III) is carried out in a suitable solvent such as THF at room temperature. In the second step of the process (III to IV) suitable reagents include s-BuLi and LDA in a solvent such as THF at a temperature between −70° C. and room temperature. The third step of the process (IV to V) is carried out by treatment with basic (such as sodium hydroxide in a suitable solvent such as methanol or alternatively ethanol) conditions and may be carried out at 120° C. under microwave condition. After the step of protection (V to VI), formula (VI) can be converted to (VII) by treatment with suitable reagent BrZn(CH₂)₃COOEt in a suitable solvent such as THF at elevated temperature under microwave condition. In the followed two steps of Suzuki coupling process (VIII to XI) suitable reagents include Pd(PPh₃)₄ and K₃PO₄ in a solvent such as DMF or DME under microwave condition are employed. The last step of the process (XI to I) is carried out by treatment with basic (such as sodium hydroxide in a suitable solvent such as isopropanol) conditions and may be carried out at room temperature. Compounds of formula (X) can be prepared as described in the experimental section.

In another aspect, compounds of formula (I) can be prepared by the process in Scheme II and Scheme III where R₂ and R₄ are as defined for formula (I):

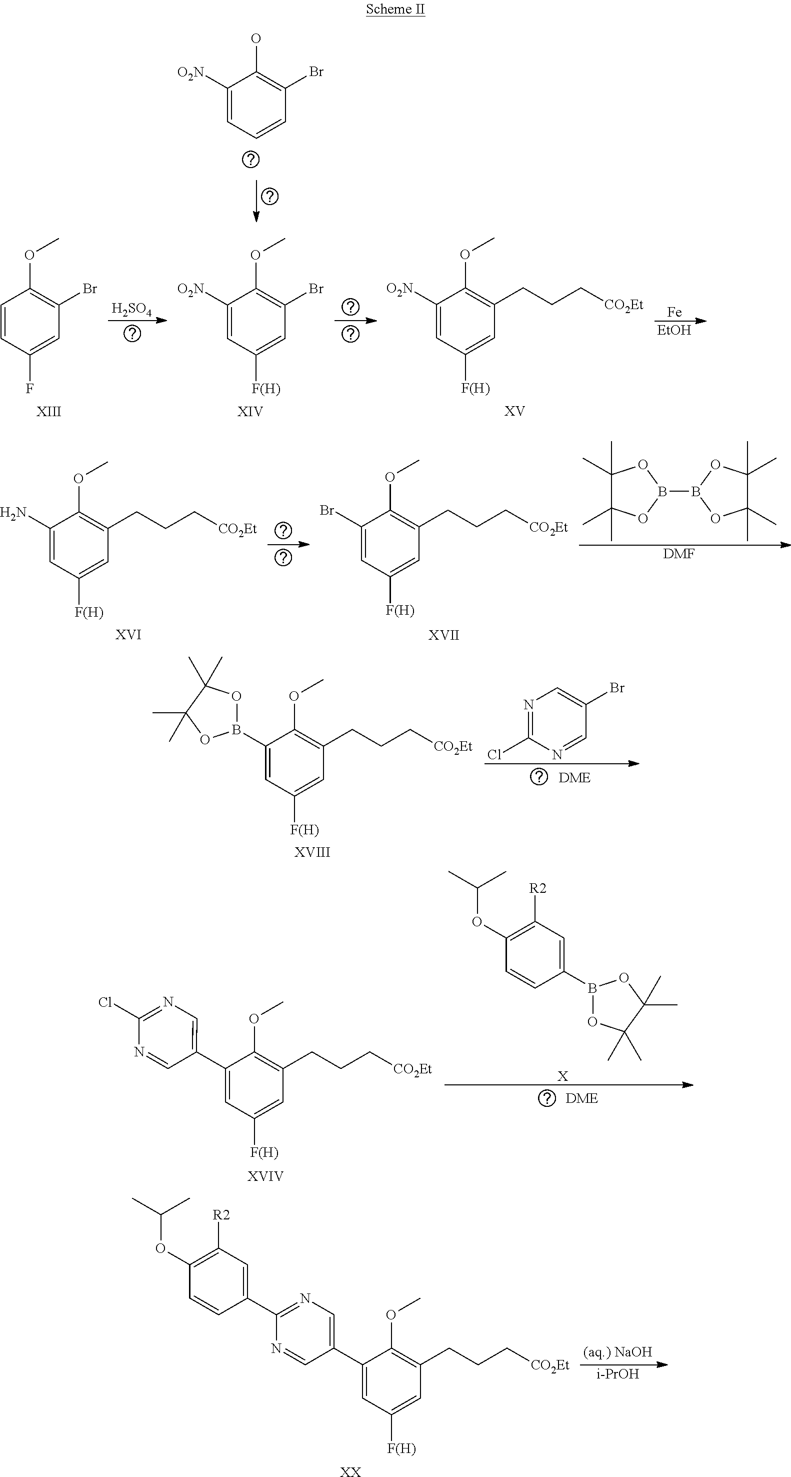

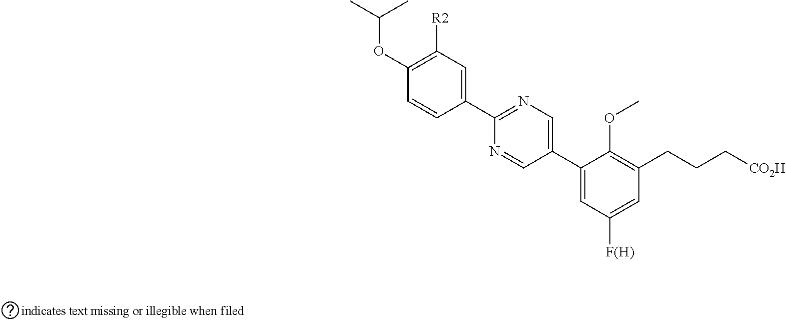

The first step of the process (XIII to XIV) is carried out with suitable reagents including sulfuric acid and nitric acid at −10° C. After the Negishi coupling (XIV to XV), XV was reduced to amine (XVI) by iron. The fourth step (XVI to XVII) is carried out in a suitable solvent such as CH₃CN. The followed processes (XVII to I) were similar with the conversion (VII to I) in scheme I.

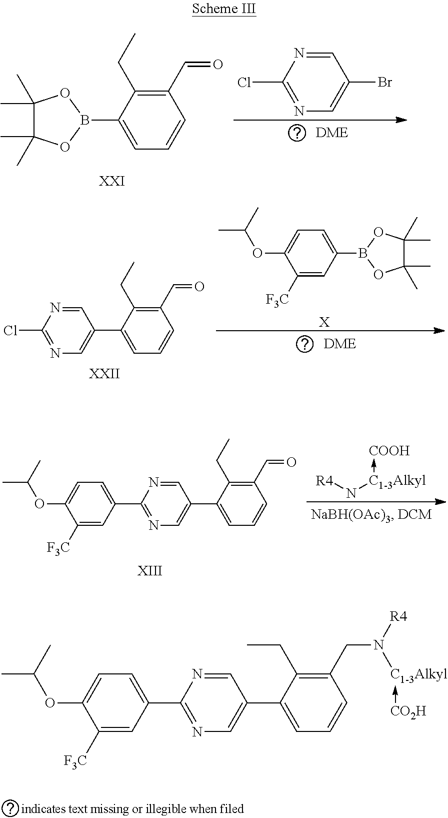

The conversion of formula XXI to XXII can be prepared followed by the steps of VIII to Xi in scheme I. The last step is carried out in suitable reagents includes amino acid and NaBH(OAc)₃ in suitable regent such as CH₂Cl₂.

In certain of the compounds of formula (I), dependent upon the nature of the substituent there are chiral carbon atoms and therefore compounds of formula (I) may exist as stereoisomers. The invention extends to all optical isomers such as stereoisomeric forms of the compounds of formula (I) including enantiomers, diastereoisomers and mixtures thereof, such as racemates. The different stereoisomeric forms may be separated or resolved one from the other by conventional methods or any given isomer may be obtained by conventional stereoselective or asymmetric syntheses.

In certain of the compounds of formula (I), dependent upon the nature of the substituent there are chiral carbon atoms and therefore compounds of formula (I) may exist as stereoisomers. The invention extends to all optical isomers such as stereoisomeric forms of the compounds of formula (I) including enantiomers, diastereoisomers and mixtures thereof, such as racemates. The different stereoisomeric forms may be separated or resolved one from the other by conventional methods or any given isomer may be obtained by conventional stereoselective or asymmetric syntheses.

Certain of the compounds herein can exist in various tautomeric forms and it is to be understood that the invention encompasses all such tautomeric forms.

Suitable compounds of formula (I) are:
4-[3-(2-{3-cyano-4-(1-methylethyl)oxy)phenyl}-5-pyrimidinyl)-2-ethylphenyl]butanoic acid
4-(2-ethyl-3-{2-[4-[(1-methylethyl)oxy]-3-(trifluoromethyl)phenyl]-5-pyrimidinyl}phenyl)butanoic acid
4-(2-ethyl-3-{2-[6-[(1-methylethyl)oxy]-5-(trifluoromethyl)-3-pyridinyl]-5-pyrimidinyl}phenyl)butanoic acid
4-[3-(2-{3-chloro-4-[(1-methylethyl)oxy]phenyl}-5-pyrimidinyl)-2-ethylphenyl]butanoic acid
4-[5-fluoro-3-{2-[4-[(1-methylethyl)oxy]-3-(trifluoromethyl)phenyl]-5-pyrimidinyl}-2-(methyloxy)phenyl]butanoic acid
4-[3-(2-{3-cyano-4-[(1-methylethyl)oxy]phenyl}-5-pyrimidinyl)-5-fluoro-2-(methyloxy)phenyl]butanoic acid
4-[3-(2-{3-cyano-4-[(1-methylethyl)oxy]phenyl}-5-pyrimidinyl)-2-(methyloxy) phenyl]butanoic acid
N-[(2-ethyl-3-{2-[4-[(1-methylethyl)oxy]-3-(trifluoromethyl)phenyl]-5-pyrimidinyl}phenyl)methyl]-N-methylglycine
N-ethyl-N-[(2-ethyl-3-{2-[4-[(1-methylethyl)oxy]-3-(trifluoromethyl)phenyl]-5-pyrimidinyl}phenyl)methyl]glycine
N-[(2-ethyl-3-{2-[4-[(1-methylethyl)oxy]-3-(trifluoromethyl)phenyl]-5-pyrimidinyl}phenyl)methyl]-b-alanine
1-[(2-ethyl-3-{2-(4-[(1-methylethyl)oxy]-3-(trifluoromethyl)phenyl]-5-pyrimidinyl}phenyl)methyl]-3-azetidinecarboxylic acid
1-{[(2-ethyl-3-{2-[4-[(1-methylethyl)oxy]-3-(trifluoromethyl)phenyl]-5-pyrimidinyl}phenyl)methyl]amino}cyclopropanecarboxylic acid
1-[(2-ethyl-3-{2-[4-[(1-methylethyl)oxy]-3-(trifluoromethyl)phenyl]-5-pyrimidinyl}phenyl)methyl]-4-piperidinecarboxylic acid
4-(3-{2-[3-cyano-4-(2-methylpropyl)phenyl]-5-pyrimidinyl}-2-ethylphenyl)butanoic acid
3-{4-[3-(2-{3-chloro-4-[(1-methylethyl)oxy]phenyl}-5-pyrimidinyl)-2-ethylphenyl]-1-piperidinyl}propanoic acid
4-{4-[3-(2-{3-chloro-4-[(1-methylethyl)oxy]phenyl}-5-pyrimidinyl)-2-ethylphenyl]-1-piperidinyl}butanoic acid
N-{[3-(2-{3-chloro-4-[(1-methylethyl)oxy]phenyl}-5-pyrimidinyl)-2-ethylphenyl]methyl}-N-methylglycine
N-{[3-(2-{3-chloro-4-[(1-methylethyl)oxy]phenyl}-5-pyrimidinyl)-2-ethylphenyl]methyl}-N-methyl-b-alanine
1-{[3-(2-{3-chloro-4-[(1-methylethyl)oxy]phenyl}-5-pyrimidinyl)-2-ethylphenyl]methyl}-3-azetidinecarboxylic acid
1-{[3-(2-{3-chloro-4-[(1-methylethyl)oxy]phenyl}-5-pyrimidinyl)-2-ethylphenyl]methyl}-4-piperidine carboxylic acid
N-[(3-{2-[3-cyano-4-(2-methylpropyl)phenyl]-5-pyrimidinyl}-2-ethylphenyl)methyl]-N-methylglycine
N-[(3-{2-[3-cyano-4-(2-methylpropyl)phenyl]-5-pyrimidinyl}-2-ethylphenyl)methyl]-N-methyl-b-alanine
1-[(3-{2-[3-cyano-4-(2-methylpropyl)phenyl]-5-pyrimidinyl}-2-ethylphenyl)methyl]-3-azetidinecarboxylic acid
1-[(3-{2-[3-cyano-4-(2-methylpropyl)phenyl]-5-pyrimidinyl}-2-ethylphenyl)methyl]-3-pyrrolidinecarboxylic acid
1-[(3-{2-[3-cyano-4-(2-methylpropyl)phenyl]-5-pyrimidinyl}-2-ethylphenyl)methyl]-3-piperidinecarboxylic acid trifluoroacetate
1-{[3-(2-{3-cyano-4-[(1-methylethyl)oxy]phenyl}-5-pyrimidinyl)-2-ethylphenyl]methyl}-3-azetidinecarboxylic acid
N-{2-[3-(2-{3-chloro-4-[(1-methylethyl)oxy]phenyl}-5-pyrimidinyl)-2-ethylphenyl]ethyl}-N-methylglycine
N-{2-[3-(2-{3-chloro-4-[(1-methylethyl)oxy]phenyl}-5-pyrimidinyl)-2-ethylphenyl]ethyl}-3-azetidinecarboxylic acid
1-{2-[3-(2-{3-chloro-4-[(1-methylethyl)oxy]phenyl}-5-pyrimidinyl)-2-ethylphenyl]ethyl}-3-pyrrolidinecarboxylic acid
(3S)-1-{2-[3-(2-{3-chloro-4-[(1-methylethyl)oxy]phenyl}-5-pyrimidinyl)-2-ethylphenyl]ethyl}-3-pyrrolidinecarboxylic acid
1-{2-[3-(2-{3-chloro-4-[(1-methylethyl)oxy]phenyl}-5-pyrimidinyl)-2-ethylphenyl]ethyl}-3-piperidinecarboxylic acid
(3S)-1-{2-[3-(2-{3-chloro-4-[(1-methylethyl)oxy]phenyl}-5-pyrimidinyl)-2-ethylphenyl]ethyl}-3-piperidinecarboxylic acid
1-{2-[3-(2-{3-chloro-4-[(1-methylethyl)oxy]phenyl}-5-pyrimidinyl)-2-ethylphenyl]ethyl}-4-piperidinecarboxylic acid
N-[2-(3-{2-[3-cyano-4-(2-methylpropyl)phenyl]-5-pyrimidinyl}-2-ethylphenyl)ethyl]-N-methylglycine
N-[2-(3-{2-[3-cyano-4-(2-methylpropyl)phenyl]-5-pyrimidinyl}-2-ethylphenyl)ethyl]-N-methyl-b-alanine
1-[2-(3-{2-{3-cyano-4-(2-methylpropyl)phenyl}-5-pyrimidinyl}-2-ethylphenyl)ethyl]-3-azetidinecarboxylic acid
1-[2-(3-{2-[3-cyano-4-(2-methylpropyl)phenyl]-5-pyrimidinyl}-2-ethylphenyl)ethyl]-3-pyrrolidinecarboxylic acid
1-[2-(3-{2-[3-cyano-4-(2-methylpropyl)phenyl]-5-pyrimidinyl}-2-ethylphenyl)ethyl]-3-piperidinecarboxylic acid
(3S)-1-[2-(3-{2-[3-cyano-4-(2-methylpropyl)phenyl]-5-pyrimidinyl}-2-ethylphenyl)ethyl]-3-piperidinecarboxylic acid
1-[2-(3-{2-[3-cyano-4-(2-methylpropyl)phenyl]-5-pyrimidinyl}-2-ethylphenyl)ethyl]-4-piperidinecarboxylic acid
4-{[3-(2-{3-chloro-4-[(1-methylethyl)oxy]phenyl}-5-pyrimidinyl)-2-ethylphenyl]oxy}butanoic acid
4-{[3-(2-{3-cyano-4-[(1-methylethyl)oxy]phenyl}-5-pyrimidinyl)-2-ethylphenyl]oxy}butanoic acid
4-[(3-{2-[3-cyano-4-(2-methylpropyl)phenyl]-5-pyrimidinyl}-2-ethylphenyl)oxy]butanoic acid
   or salts thereof.

Pharmaceutically acceptable derivatives of compounds of formula (I) include any pharmaceutically acceptable salt, ester or salt of such ester of a compound of formula (I) which, upon administration to the recipient is capable of providing (directly or indirectly) a compound of formula (I) or an active metabolic or residue thereof.

The compounds of formula (I) can form salts. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. Certain of the compounds of formula (I) may form acid addition salts with one or more equivalents of the acid. The present invention includes within its scope all possible stoichiometric and non-stoichiometric forms. Salts may also be prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glutamine, glucosamine, histidine, hydrabamine, isopropylamine, lysine, methylglucamine, morpholine, piperazine, piperidine, procaine, purines, theobromine, triethylamine, trimethylamine, tripropylamine, tris(hydroxymethyl)aminomethane (IRIS, trometamol) and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

The compounds of formula (I) may be prepared in crystalline or non-crystalline form, and, if crystalline, may optionally be hydrated or solvated. This invention includes within its scope stoichiometric hydrates or solvates as well as compounds containing variable amounts of water and/or solvent.

Included within the scope of the invention are all salts, solvates, hydrates, complexes, polymorphs, prodrugs, radiolabelled derivatives, stereoisomers and optical isomers of the compounds of formula (I).

Pharmaceutically acceptable salts may be prepared conventionally by reaction with the appropriate acid or acid derivative.

The potencies and efficacies of the compounds of this invention for the S1P1 receptor can be determined by S1P1 Tango assay performed on the human cloned receptor as described herein. Compounds of formula (I) have demonstrated agonist activity at the S1P1 receptor, using functional assays described herein.

Compounds of formula (I) and their pharmaceutically acceptable salts are therefore of use in the treatment of conditions or disorders which are mediated via the S1P1 receptor. In particular the compounds of formula (I) and their pharmaceutically acceptable salts are of use in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

Compounds of formula (I) and their pharmaceutically acceptable salts are therefore of use in the treatment of multiple sclerosis.

Compounds of formula (I) and their pharmaceutically acceptable salts may also be of use in the treatment of Parkinson's Disease, Alzheimer's disease, Huntington's chorea, amyotrophic lateral sclerosis, spinal muscular atrophy, polyglutamine expansion disorders, vascular dementia, Down's syndrome, HIV dementia, dementia, ocular diseases including glaucoma, aged related macular degeneration, cataracts, traumatic eye injury, diabetic retinopathy, traumatic brain injury, stroke, tauopathies and hearing loss.

It is to be understood that “treatment” as used herein includes prophylaxis as well as alleviation of established symptoms.

Thus the invention also provides a compound of formula (I) or a pharmaceutically acceptable salt thereof, for use as a therapeutic substance, in particular in the treatment of the conditions or disorders mediated via the S1P1 receptor. In particular the invention provides a compound of formula (I) or a pharmaceutically acceptable salt thereof for use as a therapeutic substance in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes. The invention further provides a method of treatment of conditions or disorders in mammals including humans which can be mediated via the S1P1 receptor, which comprises administering to the sufferer a therapeutically safe and effective amount of a compound of formula (I) or a pharmaceutically acceptable salt thereof.

Compounds of formula (I) and their pharmaceutically acceptable salts are of use as therapeutic substances in the treatment of multiple sclerosis.

In another aspect, the invention provides for the use of a compound of formula (I) or a pharmaceutically acceptable salt thereof in the manufacture of a medicament for use in the treatment of the conditions or disorders mediated via the S1P1 receptor

The invention provides a method of treatment of multiple sclerosis, which comprises administering to the sufferer a therapeutically safe and effective amount of a compound of formula (I) or a pharmaceutically acceptable salt thereof.

In order to use the compounds of formula (I) and pharmaceutically acceptable salts thereof in therapy, they will normally be formulated into a pharmaceutical composition in accordance with standard pharmaceutical practice. The present invention also provides a pharmaceutical composition, which comprises a compound of formula (I) or a pharmaceutically acceptable salt thereof, and a pharmaceutically acceptable carrier or excipient.

In a further aspect, the present invention provides a process for preparing a pharmaceutical composition, the process comprising mixing a compound of formula (I) or a pharmaceutically acceptable salt thereof and a pharmaceutically acceptable carrier or excipient.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents (e.g. pregelatinised maize starch, polyvinylpyrrolidone or hydroxypropyl methylcellulose); fillers (e.g. lactose, microcrystalline cellulose or calcium hydrogen phosphate); tabletting lubricants (e.g. magnesium stearate, talc or silica); disintegrants (e.g. potato starch or sodium starch glycollate); and acceptable wetting agents (e.g. sodium lauryl sulphate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound.

For parenteral administration, fluid unit dosage forms are prepared utilising a compound of the invention or pharmaceutically acceptable salts thereof and a sterile vehicle. Formulations for injection may be presented in unit dosage form e.g. in ampoules or in multi-dose, utilising a compound of the invention or pharmaceutically acceptable derivatives thereof and a sterile vehicle, optionally with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of formula (I) or pharmaceutically acceptable salts thereof may also be formulated in rectal compositions such as suppositories or retention enemas, e.g. containing conventional suppository bases such as cocoa butter or other glycerides.

The compounds of formula (I) or pharmaceutically acceptable salts thereof may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of formula (I) or pharmaceutically acceptable salts thereof, may be formulated as solutions for administration via a suitable metered or unitary dose device or alternatively as a powder mix with a suitable carrier for administration using a suitable delivery device. Thus compounds of formula (I) or pharmaceutically acceptable salts thereof may be formulated for oral, buccal, parenteral, topical (including ophthalmic and nasal), depot or rectal administration or in a form suitable for administration by inhalation or insufflation (either through the mouth or nose).

The compounds of formula (I) or pharmaceutically acceptable salts thereof may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, 1.0 to 500 mg or 1.0 to 200 mg and such unit doses may be administered more than once a day, for example two or three times a day.

Compounds of formula (I) or pharmaceutically acceptable salts thereof may be used in combination preparations. For example, the compounds of the invention may be used in combination with cyclosporin A, methotrexate, steriods, rapamycin, proinflammatory cytokine inhibitors, immunomodulators including biologicals or other therapeutically active compounds.

The subject invention also includes isotopically-labeled compounds, which are identical to those recited in formulas I and following, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹⁸F ¹²³I and ¹²⁵I.

Compounds of the present invention and pharmaceutically acceptable salts of said compounds that contain the aforementioned isotopes and/or other isotopes of other atoms are within the scope of the present invention. Isotopically-labeled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula (I) and following of this invention can generally be prepared by carrying out the procedures disclosed in the Schemes and/or in the Examples below, by substituting a readily available isotopically labelled reagent for a non-isotopically labeled reagent.

In a further aspect, this invention provides processes for preparation of a compound of formula (I).

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of compounds of the invention.

### Abbreviations

g—grams
mg—milligrams
ml—millilitres
min—minute
ul—microlitres
MeCN—acetonitrile
MeOH—methanol
EtOH—ethanol
Et₂O—diethyl ether
EtOAc—ethyl acetate
DABCO—1,4-diazabiclo[2,2,2]octane
DCM—dichloromethane
DIAD—diisopropyl azodicarboxylate
DME—1,2-bis(methyloxy)ethane

### DMF—N,N-dimethylformamide

DMSO—dimethylsulphoxide
EDAC—N-(3-Dimethylaminopropyl)-W-ethylcarbodiimide hydrochloride
EDC—N-(3-Dimethylaminopropyl)-AP-ethylcarbodiimide hydrochloride
EDCl—N-(3-Dimethylaminopropyl)-N′-ethylcarbodiimide hydrochloride

### HOBT/HOBt—Hydroxybenzotriazole

IPA—isopropylalcohol

### NCS—N-chlorosuccinimide

PyBOP—Benzotriazol-1-yl-oxytripyrrolidinophosphonium hexafluorophosphate
THF—tetrahydrofuran
dba—dibenzylidene acetone
RT—room temperature
° C.—degrees Celsius

### M—Molar

H—proton
s—singlet
d—doublet
t—triplet
q—quartet
MHz—megahertz
MeOD—deuterated methanol

### LCMS—Liquid Chromatography Mass Spectrometry

### LC/MS—Liquid Chromatography Mass Spectrometry

MS—mass spectrometry

### ES—Electrospray

MH⁺—mass ion+H⁺
MDAP—mass directed automated preparative liquid chromatography.
sat.—saturated

### General Chemistry Section

The intermediates for the preparation of the examples may not necessarily have been prepared from the specific batch of precursor described.

### Chromatography

Unless stated otherwise, all chromatography was carried out using silica columns.

### LCMS

### 1) Acidic Condition:

Mobile phase: water containing 0.05% TFA/acetonitrile

Column: XBridge™ C18 30×100 mm-5 microns

Detection: MS and photodiode array detector (PDA)

### 2) Basic Condition:

Mobile phase: water containing 0.08% NH₄HCO₃/acetonitrile

Column: XBridge™ C18 30×100 mm-5 microns;

Detection: MS and photodiode array detector (PDA)

### MDAP

1) acidic Condition 1:

Instrument: Waters instrument

Column: Sunfire Prep C18 column (5 um, 19×50 mm)

Mobile phase: water containing 0.05% TFA/acetonitrile.

2) acidic Condition 2:

Instrument: Gilson GX-281

Column: Sunfire prep C18 OBD; 5 um, 100 mm*30 mm;

Mobile phase: A: 0.05% TFA/H₂O; B: MeCN;

### 3) Basic Condition 1:

Instrument: Waters instrument

Column: Xbridge Prep C18 column (5 um, 19×50 mm)

Mobile phase: water containing 0.04% ammonia/acetonitrile.

4) basic condition 2:

Instrument: Gilson 281(PHG-005);

Column: Shimadzu PRC-ODS 20×250 mm, 15 um two connected in series;

Mobile phase: A: 10 mM NH₄HCO₃ B:MeCN;

5) basic Condition 3:

Instrument: Gilson GX-281;

Column: Agela Durashell RP 21.5*250 mm 10 μm;

Mobile phase: A: 0.04% NH₃H₂O/water; CH₃CN;

### Description for D1

3-chlorophenyl diethylcarbamate (D1)

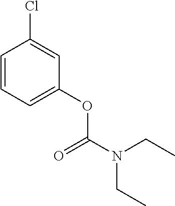

To a solution of 3-chlorophenol (10 g) in tetrahydrofuran (THF) (100 mL) was added NaH (6.22 g) at room temperature. The result suspension was stirred for 2 h. Diethylcarbamic chloride (21.09 g) was added and the reaction mixture was stirred for another 3 h. The reaction was quenched with water (10 mL), washed with brine (20 mL) for 3 times. The organic phase was concentrated and the residue was purified by column chromatography to give 3-chlorophenyl diethylcarbamate (D1) (14.2 g) as a light oil. δH (CDCl₃, 400 MHz): 1.24 (6H, m), 3.41 (4H, m), 7.05 (1H, m), 7.19 (2H, m), 7.27 (1H, m). MS (ES): C₁ᵢH₁₄ClNO₂ requires 227; found 228.1 (M+H⁺).

### Description for D2

3-chloro-2-ethylphenyl diethylcarbamate (D2)

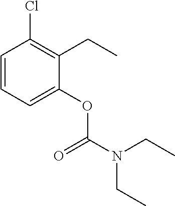 [SMILES: CCc1c(Cl)cccc1OC(=O)N(CC)CC]

To a solution of TMEDA (16.94 mL) and 3-chlorophenyl diethylcarbamate (D1) (14.2 g) in dry tetrahydrofuran (THF) (25 mL) at −70° C. was added sec-butyllithium (86 mL). The reaction mixture was stirred at this temperature for 2 h. Ethyl iodide (15.12 mL) was added and the reaction mixture was stirred at −70° C. for 1 hour. Then the reaction mixture was warmed to room temperature and stirred overnight. The reaction was quenched with saturated aqueous NH₄Cl (10 mL) and partitioned between brine (10 mL) and ethyl acetate (50 mL). The organic phase was concentrated and the residue was purified by column chromatography to give 3-chloro-2-ethylphenyl diethylcarbamate (D2) (11.7 g) as a light yellow oil. MS (ES): C₁₃H₁₈ClNO₂ requires 255; found 256.1 (M+H⁺).

### Description for D3

3-chloro-2-ethylphenol (D3)

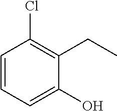

To a solution of 3-chloro-2-ethylphenyl diethylcarbamate (D2) (2.5 g) in ethanol (8 mL) was added NaOH (2.5 g) at room temperature. The reaction vessel was sealed and heated under microwave at 120° C. for 90 min. The mixture was concentrated to remove the solvent and the pH value was adjusted to about 6 with conc. HCl and 2M Ha under ice bath. The mixture was partitioned between ethyl acetate and brine. The organic phase was concentrated to give 3-chloro-2-ethylphenol (D3) (990 mg) as a light brown oil. δH (DMSO-d₆, 400 MHz): 1.09 (3H, t), 2.71 (2H, m), 6.80 (1H, d), 6.85 (1H, d), 7.03 (1H, t), 9.80 (1H, s).

### Description for D4

3-chloro-2-ethylphenyl trifluoromethanesulfonate (D4)

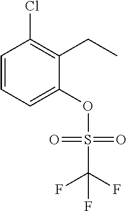 [SMILES: CCc1c(Cl)cccc1OS(=O)(=O)C(F)(F)F]

To the mixture of 3-chloro-2-ethylphenol (D3) (12.4 g) and DMAP (13.54 g) in dichloromethane (DCM) (50 mL) was added 1,1,1-trifluoro-N-phenyl-N-[(trifluoromethyl)sulfonyl]methanesulfonamide (28.3 g). The reaction mixture was stirred at room temperature overnight. Water (1 mL) was added and the mixture was stirred for 2 min. The resulting mixture was concentrated to remove the solvent. Petroleum ether (50 mL) was added and most of suspension emerged. The mixture was filtered, the filtrate was concentrated and the residue was purified by column chromatography to give 3-chloro-2-ethylphenyl trifluoromethanesulfonate (D4) (10.0 g). δH (CDCl₃, 400 MHz): 1.15 (3H, t), 2.80 m), 7.14 (2H, m), 7.33 (1H, m).

### Description for D5

ethyl 4-(3-chloro-2-ethylphenyl)butanoate (D5)

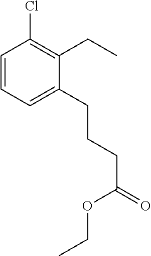 [SMILES: CCOC(=O)CCCc1cccc(Cl)c1CC]

To the mixture of Pd₂(dba)₃ (0.095 g) and 1,1′-bis(diphenylphosphino)ferrocene (0.058 g) in tetrahydrofuran (THE) (8 mL) was added 3-chloro-2-ethylphenyl trifluoromethanesulfonate (D4) (1.5 g) and bromo[4-(ethyloxy)-4-oxobutyl]zinc (18.71 mL) under nitrogen at room temperature. The reaction vessel was sealed and heated under microwave at 120° C. for 45 min, The reaction was quenched with water, the mixture was concentrated in vacuo and the residue was purified by column chromatography to give ethyl 4-(3-chloro-2-ethylphenyl)butanoate (D5) (0.66 g) as a brown oil. MS (ES): C₁₄H₁₉ClO₂ requires 254; found 255.1 (M+H⁺).

### Description for D6

ethyl 4-[2-ethyl-3-(4,4,5,5-tetramethyl-1,3,2-dioxaborolan-2-yl)phenyl]butanoate (D6)

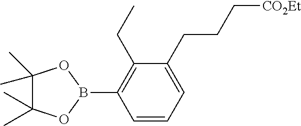 [SMILES: CCOC(=O)CCCc1cccc(B2OC(C)(C)C(C)(C)O2)c1CC]

Tricyclohexylphosphine (0.550 g) and Pd₂(dba)₃ (0.144 g) in N,N-dimethylformamide (DMF) (12 mL) were stirred under nitrogen for 30 min. Ethyl 4-(3-chloro-2-ethylphenyl)butanoate (D5) (1 g), 4,4,4′,4′,5,5,5′,5′-octamethyl-2,2′-bi-1,3,2-dioxaborolane (1.794 g) and potassium acetate (0.770 g) were added to the reaction mixture under nitrogen at room temperature. The reaction vessel was sealed and heated under microwave at 180° C. for 90 min. After cooling the reaction, the reaction was filtered, the filtrate was concentrated in vacuo and the residue was purified by column chromatography to give ethyl 4-[2-ethyl-3-(4,4,5,5-tetramethyl-1,3,2-dioxaborolan-2-yl)phenyl]butanoate (D6) (0.617 g) as a brown oil. δH (CDCl₃, 400 MHz): 1.15 (3H, t), 1.28 (3H, t), 1.36 (12H, s), 1.92 (2H, m), 2.38 (2H, t), 2.68 (2H, t), 2.96 (2H, m), 4.15 (2H, m), 7.13 (1H, m), 7.21 (1H, d), 7.63 (1H, d). MS (ES): C₂₀H₃₁BO₄ requires 346; found 347.3 (M+H⁺).

### Description for D7

ethyl 4-[3-(2-chloro-5-pyrimidinyl)-2-ethylphenyl]butanoate (D7)

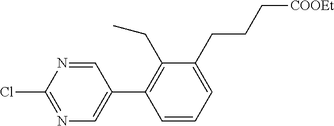 [SMILES: CCOC(=O)CCCc1cccc(-c2cnc(Cl)nc2)c1CC]

To a solution of in ethyl 4-[2-ethyl-3-(4,4,5,5-tetramethyl-1,3,2-dioxaborolan-2-yl)phenyl]butanoate (D6) (500 mg), tripotassium phosphate (613 mg) and 5-bromo-2-chloropyridine (559 mg) in N,N-dimethylformamide (DMF) (4 mL) and water (1 mL) under nitrogen at room temperature was added Pd(Ph₃P)₄ (167 mg) in one charge. The reaction vessel was sealed and heated under microwave at 120° C. for 10 min. After cooling the reaction, the reaction mixture was concentrated in vacuo and the residue was purified by column chromatography to give ethyl 4-[3-(2-chloro-5-pyrimidinyl)-2-ethylphenyl]butanoate (D7) (200 mg) as a brown oil. MS (ES): C₁₈H₂₁ClN₂O₂ requires 332; found 333.1 (M+H⁺).

### Description for D8

5-bromo-2[(1-methylethyl)oxy]benzonitrile (D8)

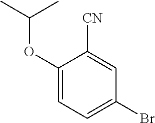

To a solution of 5-bromo-2-hydroxybenzonitrile (25 g) in acetonitrile (150 mL) was added 2-iodopropane (15.14 mL) and potassium carbonate (34.9 g). The reaction mixture was stirred at room temperature for two days. The solvent was removed in vacuo, the residue was dissolved in ethyl acetate (150 mL), washed with water (2*30 mL), the organic phase was dried over sodium sulphate and concentrated to afford 5-bromo-2-[(1-methylethyl)oxy]benzonitrile (D8) (29.8 g) as a white solid without further purification. δH (CDCl₃, 400 MHz): 1.39 (6H, d), 4.61 (1H, m), 6.85 (1H, d), 7.58 (1H, dd), 7.64 (1H, d). MS (ES): C₁₀H₁₀BrNO requires 239; found 240.0 (M+H⁺).

### Description for D9

2-[(1-methylethyl)oxy]-5-(4,4,5,5-tetramethyl-1,3,2-dioxaborolan-2-yl)benzonitrile (D9)

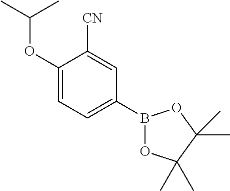 [SMILES: CC(C)Oc1ccc(B2OC(C)(C)C(C)(C)O2)cc1C#N]

To a suspension of 5-bromo-2-[(1-methylethyl)oxy]benzonitrile (D8) (123 mg), 4,4,4′,″, 5,5,5′,5′-octamethyl-2,2′-bi-1,3,2-dioxaborolane (156 mg) and potassium acetate (101 mg) in N,N-dimethylformamide (DMF) (150 mL) stirred under nitrogen at room temperature was added PdCl₂(dppf)-CH₂Cl₂ adduct (25.1 mg). The reaction vessel was sealed and heated under microwave at 120° C. for 1 h. After cooling the reaction, the reaction mixture was concentrated in vacuo and the residue was purified by column chromatography to give 2-[(1-methylethyl)oxy]-5-(4,4,5,5-tetramethyl-1,3,2-dioxaborolan-2-yl)benzonitrile (D9) (54 mg). MS (ES): C₁₆H₂₂BNO₃ requires 287; found 288.2 (M+H⁺).

### Description for D10

4-bromo-2-chloro-1-[(1-methylethyl)oxy]benzene (D10)

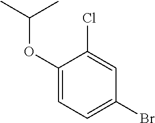

To a solution of 4-bromo-2-chlorophenol (50 g) in N,N-dimethylformamide (DMF) (250 mL) stirred under nitrogen at room temperature was added K₂CO₃ (100 g) and 2-bromopropane (136 mL) in one charge. The reaction mixture was stirred at 85° C. for 16 h. After cooling the reaction, the reaction mixture was filtered, the solvent of the filtrate was removed in vacuo. The residue was dissolved in diethyl ether (300 mL), washed with water (6*100 mL), the organic phase was dried over MgSO₄ and concentrated to give 4-bromo-2-chlorophenyl 1-methylethyl ether (D10) (56 g) as a yellow oil. δH (CDCl₃, 400 MHz): 1.37 (6H, d), 4.52 (1H, m), 6.82 (1H, d), 7.29 (1H, m), 7.50 (1H, d).

### Description for D11

2-{3-chloro-4-[(1-methylethyl)oxy]phenyl}-4,4,5,5-tetramethyl-1,3,2-dioxaborolane (D11)

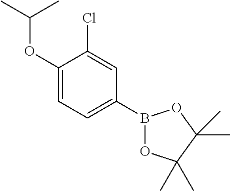 [SMILES: CC(C)Oc1ccc(B2OC(C)(C)C(C)(C)O2)cc1Cl]

To a suspension of 4-bromo-2-chloro-1-[(1-methylethyl)oxy]benzene (D10) (10 g), 4,4,4′,4′,5,5,5′,5′-octamethyl-2,2′-bi-1,3,2-dioxaborolane (15.26 g) and potassium acetate (15.73 g) in N,N-dimethylformamide (DMF) (150 mL) stirred under nitrogen at room temperature was added PdCl₂(dppf)-CH₂Cl₂ adduct (1.964 g). The reaction mixture was stirred at 80° C. overnight. After cooling the reaction, the reaction mixture was concentrated in vacuo, the residue was diluted with ethyl acetate and filtered through celite, the filtrate was washed with water and brine, the organic phase was dried over anhydrous Na₂SO₄. After removing the solvent, the residue was purified by column chromatography to give 2-{3-chloro-4-[(1-methylethyl)oxy]phenyl}-4,4,5,5-tetramethyl-1,3,2-dioxaborolane (D11) (11.8 g). MS (ES): C₁₅H₂₂BClO₃ requires 296; found 297.1 (M+H⁺).

### Description for D12

4-bromo-1-fluoro-2-(trifluoromethyl)benzene (D12)

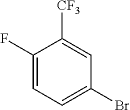 [SMILES: Fc1ccc(Br)cc1C(F)(F)F]

To a suspension of copper(II) bromide (7.48 g) in acetonitrile (50 mL) was added 1,1-dimethylethyl nitrite (5.02 mL) dropwise under ice-cooling, the mixture was stirred under nitrogen for 5 min. A solution of 4-fluoro-3-(trifluoromethyl)aniline (5 g) in acetonitrile was added to the reaction mixture under ice-cooling, the mixture was stirred at room temperature under nitrogen for 2 h. To the resulting suspension was added 1N HCl and the reaction mixture was concentrated in vacuo. The residue was diluted with water and extracted with ethyl acetate, the combined organic phases were dried over sodium sulphate and concentrated, the residue was purified by column chromatography to give 4-bromo-1-fluoro-2-(trifluoromethyl)benzene (D12) (2 g). δH (CDCl₃, 400 MHz): 7.11 (1H, t), 7.66 (1H, m), 7.74 (1H, dd). δF (CDCl₃, 376 MHz): −116.2, −61.7.

### Description for D13

4-bromo-1-[(1-methylethyl)oxy]-2-(trifluoromethyl)benzene (D13)

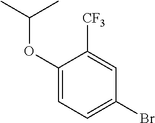

To a solution of 2-propanol (1.997 mL) in dry tetrahydrofuran (THF) (50 mL) under nitrogen was added potassium tert-butoxide (3.49 g). The reaction mixture was heated to 50° C. for 10 min, then 4-bromo-1-fluoro-2-(trifluoromethyl)benzene (D12) (6.3 g) was added. The resulting mixture was stirred at 50° C. overnight. After cooling the reaction, the solvent was removed in vacuo, the residue was diluted with ethyl acetate (100 mL), washed with water, the organic phase was dried over sodium sulphate, concentrated and the residue was purified by column chromatography to give 4-bromo-1-[(1-methylethyl)oxy]-2-(trifluoromethyl)benzene (D13) (5.21 g) as a clear oil. δH (CDCl₃, 600 MHz): 1.36 (6H, d), 4.60 (1H, m), 6.88 (1H, d), 7.55 (1H, dd), 7.66 (1H, d).

### Description for D14

4,4,5,5-tetramethyl-2-[4-[(1-methylethyl)oxy]-3-(trifluoromethyl)phenyl]-1,3,2-dioxaborolane (D14)

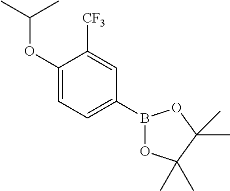 [SMILES: CC(C)Oc1ccc(B2OC(C)(C)C(C)(C)O2)cc1C(F)(F)F]

To a solution of 4-bromo-1-[(1-methylethyl)oxy]-2-(trifluoromethyl)benzene (D13) (5.21 g) in dry tetrahydrofuran (THF) (50 mL) was added n-BuLi (12.65 mL) dropwise at −78° C. (maintaining the temperature <−60° C.). The resulting solution was stirred at −78° C. for 30 min before triisopropyl borate (5.13 mL) was added dropwise (<−60° C.). The reaction mixture was allowed to warm to room temperature, then pinacol (0.70 g) and AcOH (2.107 mL) was added and the reaction mixture stirred at room temperature overnight. The reaction was quenched with saturated aqueous NH₄Cl, the mixture was diluted with ethyl acetate and washed with saturated aqueous NaHCO₃ and brine, the organic phase was dried over sodium sulphate and concentrated. The residue was purified by column chromatography to give 4,4,5,5-tetramethyl-2-[4-[(1-methylethyl)oxy]-3-(trifluoromethyl)phenyl]-1,3,2-dioxaborolane (D14) (5.3 g) as a clear oil. δH (CDCl₃, 600 MHz): 1.33 (12H, s), 1.36 (6H, d), 4.69 (1H, m), 6.97 (1H, d), 7.88 (1H, d), 7.99 (1H, s). MS (ES): C₁₋₅H₂₂BF₃O₃ requires 330; found 331.2 (M÷H⁺).

### Description for D15

3-(trifluoromethyl)-2(1H)-pyridinone (D15)

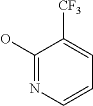 [SMILES: Oc1ncccc1C(F)(F)F]

To a solution of 2-chloro-3-(trifluoromethyl)pyridine (10 g) in tert-Butanol (20 mL) at room temperature was added KOH (6.18 g). The reaction mixture was stirred at 90° C. overnight. The reaction was neutralized with HOAc, the resulting mixture was extracted with ethyl acetate, the combined organic phases were dried over sodium sulphate and concentrated to give 3-(trifluoromethyl)-2(1H)-pyridinone (D15) (5.8 g). MS (ES): C₅H₄F₃NO requires 163; found 164.1 (M+H⁺).

### Description for D16

5-bromo-3-(trifluoromethyl)-2(1H)-pyridinone (D16)

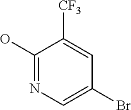 [SMILES: Oc1ncc(Br)cc1C(F)(F)F]

To a solution of 3-(trifluoromethyl)-2(1H)-pyridinone (D15) (10 g) in tetrahydrofuran (THF) (100 mL) stirred in air at room temperature was added NBS (14.19 g) portionwise. The reaction mixture was stirred for 4 h, then diluted with water (100 mL), extracted with ethyl acetate (3*50 mL). The combined organic phases were dried over sodium sulphate and concentrated to give 5-bromo-3-(trifluoromethyl)-2(1H)-pyridinone (D16) (14 g) as yellow solid. MS (ES): C₆H₃BrF₃NO requires 241; found 242.0 (M+H⁺).

### Description for D17

5-bromo-2-[(1-methylethyl)oxy]-3-(trifluoromethyl)pyridine (D17)

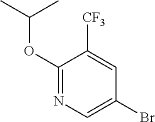

To a solution of 5-bromo-3-(trifluoromethyl)-2(1H)-pyridinone (D16) (2 g) and silver carbonate (6.84 g) in toluene (25 mL) stirred in air at room temperature was added 2-iodopropane (5.79 mL) dropwise. The reaction mixture was stirred at room temperature for 1 day. The reaction mixture was filtered and the filtrate was concentrated, the residue was purified by column chromatography to give 5-bromo-2-[(1-methylethyl)oxy]-3-(trifluoromethyl)pyridine (D17) (1.2 g).

### Description for D18

2-[(1-methylethyl)oxy]-5-(4,4,5,5-tetramethyl-1,3,2-dioxaborolan-2-yl)-3-(trifluoromethyl)pyridine (D18)

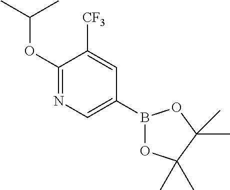 [SMILES: CC(C)Oc1ncc(B2OC(C)(C)C(C)(C)O2)cc1C(F)(F)F]

To a solution of 5-bromo-2-[(1-methylethyl)oxy]-3-(trifluoromethyl)pyridine (D17) (828 mg) in dry tetrahydrofuran (THF) (10 mL) stirred under nitrogen at −78° C. was added n-BuLi (2.004 mL) dropwise over 1 min. The reaction mixture was stirred at −78° C. for 20 min, then triisopropyl borate (0.812 mL) was added dropwise in 30 sec. After 20 min, the reaction mixture was allowed to warm to room temperature, then pinacol (413 mg) was added followed by AcOH (0.334 mL). The mixture was stirred overnight. The reaction was quenched with saturated aqueous NH₄Cl, the mixture was diluted with ethyl acetate and washed with saturated aqueous NaHCO₃ and brine, the organic phase was dried over sodium sulphate and concentrated. The residue was purified by column chromatography to give 2-[(1-methylethyl)oxy]-5-(4,4,5,5-tetramethyl-1,3,2-dioxaborolan-2-yl)-3-(trifluoromethyl)pyridine (D18) (438 mg) as a yellow oil. MS (ES): C₁₅H₂₁BF₃NO₃ requires 331; found 332.2 (M+H⁺).

### Description for D19

Ethyl 4-[3-(2-{3-cyano-4-[(1-methylethyl)oxy]phenyl}-5-pyrimidinyl)-2-ethylphenyl]butanoate (D19)

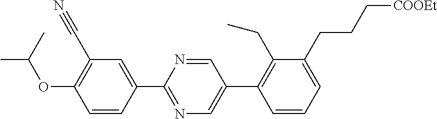 [SMILES: CCOC(=O)CCCc1cccc(-c2cnc(-c3ccc(OC(C)C)c(C#N)c3)nc2)c1CC]

To a solution of ethyl 4-[3-(2-chloro-5-pyrimidinyl)-2-ethylphenyl]butanoate (D7) (96 mg), 2-[(1-methylethyl)oxy]-5-(4,4,5,5-tetra methyl-1,3,2-dioxaborolan-2-yl) Benzonitrile (D9) (83 mg) and tripotassium phosphate (122 mg) in N,N-dimethylformamide (DMF) (4 mL) and water (1 mL) stirred under nitrogen at room temperature was added Pd(Ph₃P)₄ (33.3 mg) in one charge. The reaction vessel was sealed and heated under microwave at 130° C. for 10 min. After cooling the reaction, the reaction mixture was concentrated and the residue was purified by column chromatography to give ethyl 4-[3-(2-{3-cyano-4-[(1-methylethyl)oxy]phenyl}-5-pyrimidinyl)-2-ethylphenyl]butanoate (D19) (65 mg) as a light oil. MS (ES): C₂₈H₃₁N₃O₃ requires 457; found 458.3 (M+H⁺).

### Description for D20

ethyl4-(2-ethyl-3-{2-[4-[(1-methylethyl)oxy]-3-(trifluoromethyl)phenyl]-5-pyrimidinyl}phenyl)butanoate (D20)

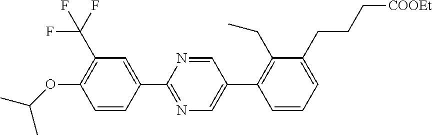 [SMILES: CCOC(=O)CCCc1cccc(-c2cnc(-c3ccc(OC(C)C)c(C(F)(F)F)c3)nc2)c1CC]

To a solution of ethyl 4-[3-(2-chloro-5-pyrimidinyl)-2-ethylphenyl]butanoate (D7) (70 mg), 4,4,5,5-tetramethyl-2-[4-[(1-methylethyl)oxy]-3-(trifluormethyl)phenyl]-1,3,2-dioxaborolane (D14) (69.4 mg) and tripotassium phosphate (112 mg) in 1,2-dimethoxyethane (DME) (5 mL) and water (1.250 mL) stirred under nitrogen at room temperature was added Pd(Ph₃P)₄ (24.30 mg, 0.021 mmol) in one charge. The reaction vessel was sealed and heated under microwave at 120° C. for 15 min. After cooling the reaction, the reaction mixture was filtered and the filtrate was partitioned between ethyl acetate 250 mL and saturated brine 50 mL. The organic phase was dried over sodium sulphate and evaporated in vacuo to give the crude product ethyl 4-(2-ethyl-3-{2-[4-[(1-methylethyl)oxy]-3-(trifluoromethyl)phenyl}-5-pyrimidinyl]phenyl)butanoate (D20) (105 mg) without further purification. MS (ES): C₂₈H₃₁F₃N₂O₃ requires 500; found 501.3 (M+H⁺).

### Description for D21

ethyl 4-(2-ethyl-3-{2-[6-[(1-methylethyl)oxy]-5-(trifluoromethyl)-3-pyridinyl]-5-pyrimidinyl}phenyl)butanoate (D21)

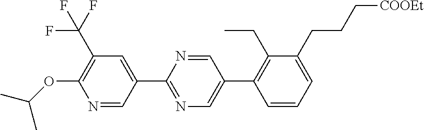 [SMILES: CCOC(=O)CCCc1cccc(-c2cnc(-c3cnc(OC(C)C)c(C(F)(F)F)c3)nc2)c1CC]

To a solution of ethyl 4-[3-(2-chloro-5-pyrimidinyl)-2-ethylphenyl]butanoate (D7) (65.3 mg), 2-[(1-methylethyl)oxy]-5-(4,4,5,5-tetramethyl-1,3,2-dioxaborolan-2-yl)-3-(trifluoromethyl)pyridine (D18) (65 mg) and tripotassium phosphate (104 mg) in 1,2-dimethoxyethane (DME) (3 mL) and water (0.750 mL) stirred under nitrogen at room temperature was added Pd(Ph₃P)₄ (22.68 mg) in one charge. The reaction vessel was sealed and heated under microwave at 130° C. for 15 min. After cooling the reaction, the reaction mixture was filtered and the filtrate was partitioned between ethyl acetate (50 mL) and saturated brine (20 mL). The organic phase was dried over sodium sulphate and evaporated in vacuo, the residue was purified by column chromatography to afford ethyl 4-(2-ethyl-3-{2-[6-[(1-methylethyl)oxy]-5-(trifluoromethyl)-3-pyridinyl]-5-pyrimidinyl}phenyl)butanoate (D21) (65 mg). MS (ES): C₂₇H₃₀F₃N₃O₃ requires 501; found 502.3 (M+H⁺).

### Description for D22

ethyl 4-[3-(2-{3-chloro-4-[(1-methylethyl)oxy]phenyl}-5-pyrimidinyl)-2-ethylphenyl]butanoate (D22)

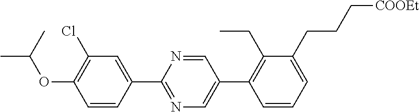 [SMILES: CCOC(=O)CCCc1cccc(-c2cnc(-c3ccc(OC(C)C)c(Cl)c3)nc2)c1CC]

To a solution of {3-chloro-4-[(1-methylethyl)oxy]phenyl}boronic acid (58.6 mg), ethyl 4-[3-(2-chloro-5-pyrimidinyl)-2-ethylphenyl]butanoate (D7) (70 mg) and cesium carbonate (171 mg) in 1,2-dimethoxyethane (DME) (3 mL) and water (0.600 mL) stirred under nitrogen at room temperature was added Pd(Ph₃P)₄ (24.30 mg) in one charge. The reaction vessel was sealed and heated under microwave at 130° C. for 15 min. After cooling the reaction, the reaction mixture was filtered and the filtrate was partitioned between ethyl acetate (100 mL) and saturated brine (50 mL). The organic phase was dried over sodium sulphate and evaporated in vacuo to give the crude product ethyl 4-[3-(2-{3-chloro-4-[(1-methylethyl)oxy]phenyl}-5-pyrimidinyl)-2-ethylphenyl]butanoate (D22) (220 mg) without further purification. MS (ES): C₂₇H₃₁ClN₂O₃ requires 466; found 467.2 (M+H⁺).

### Description for D23

1-bromo-5-fluoro-2-(methyloxy)-3-nitrobenzene (D23)

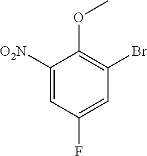

To a stirred solution of 2-bromo-4-fluoro-1-(methyloxy)benzene 2-bromo-4-fluorophenyl methyl ether (8 g) in sulfuric acid (31.2 ml) at −10° C. was added nitric acid (2.79 ml) dropwise. After stirring for 30 min, the mixture was poured into ice, extracted with EtOAc (4*50 mL). The combined organic phases were washed with brine, dried over Na₂SO₄ and concentrated, the residue was purified by column chromatography to afford 1-bromo-5-fluoro-2-(methyloxy)-3-nitrobenzene (D23) (4.09 g). δH (CDCl₃, 400 MHz): 4.00 (3H, t), 7.55 (2H, m).

### Description for D24

ethyl 4-[5-fluoro-2-(methyloxy)-3-nitrophenyl]butanoate (D24)

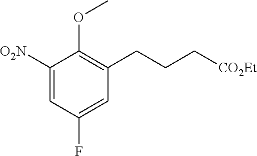 [SMILES: CCOC(=O)CCCc1cc(F)cc([N+](=O)[O-])c1OC]

To a suspension of 1-bromo-5-fluoro-2-(methyloxy)-3-nitrobenzene (D23) (4.09 g), cesium carbonate (2.132 g) and tri-t-butylphosphine (1.898 g) in tetrahydrofuran

(THF) (30 mL) under nitrogen was added bromo[4-(ethyloxy)-4-oxobutyl]zinc (65.4 mL) followed by Pd₂ dba₃ (1.498 g). The reaction mixture was stirred at room temperature overnight. Saturated aqueous ammonium chloride solution was added to quench the reaction. The aqueous phase was extracted with ethyl acetate. The combined organic phases were dried over anhydrous sodium sulphate and concentrated, the residue was purified by column chromatography to give ethyl 4-[5-fluoro-2-(methyloxy)-3-nitrophenyl]butanoate (D24) (3.64 g). MS (ES): C₁₃H₁₆FNO₅ requires 285; found 286.1 (M+H⁺).

### Description for D25

ethyl 4-[3-amino-5-fluoro-2-(methyloxy)phenyl]butanoate (D25)

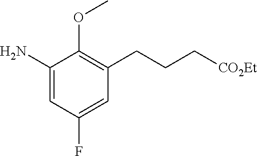 [SMILES: CCOC(=O)CCCc1cc(F)cc(N)c1OC]

A mixture of ethyl 4-[5-fluoro-2-(methyloxy)-3-nitrophenyl]butanoate (D24) (4.19 g), iron (8.20 g) and saturated aqueous ammonium chloride solution (20 mL) was stirred at 90° C. overnight. After cooling the reaction, the reaction mixture was filtered and the filtrate was evaporated to remove organic solvent, the residue was extracted with ethyl acetate. The combined organic phases were dried over anhydrous sodium sulphate and concentrated, the residue was purified by column chromatography to give ethyl 4-[3-amino-5-fluoro-2-(methyloxy)phenyl]butanoate (D25) (2.27 g). MS (ES): C₁₃H₁₈FNO₃ requires 255; found 256.2 (M+H⁺).

### Description for D26

ethyl 4-[3-bromo-5-fluoro-2-(methyloxy)phenyl]butanoate (D26)

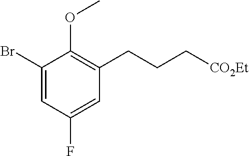 [SMILES: CCOC(=O)CCCc1cc(F)cc(Br)c1OC]

To a solution of ethyl 4-[3-amino-5-fluoro-2-(methyloxy)phenyl]butanoate (D25) (1.7 g) in acetonitrile (50 mL) was added HBr (7.23 mL) at 0° C. Then a solution of sodium nitrite (0.919 g) in water (5 mL) was added to the reaction mixture. After stirring for 10 min, copper(II) bromide (2.97 g) and copper(I) bromide (0.191 g) were added, The reaction mixture was stirred at 50° C. for 1 h. After cooling the reaction, aqueous NH₄Cl solution was added, the resulting suspension was extracted with ethyl acetate. The combined organic phases were dried over anhydrous sodium sulphate and concentrated. The residue was purified by column chromatography to give ethyl 4-[3-bromo-5-fluoro-2-(methyloxy)phenyl]butanoate (D26) (1.84 g). MS (ES): C₁₃H₁₆BrFO₃ requires 318; found 319.1 (M+H⁺).

### Description for D27

Ethyl 4-[5-fluoro-2-(methyloxy)-3-(4,4,5,5-tetramethyl-1,3,2-dioxaborolan-2-yl)phenyl]butanoate (D27)

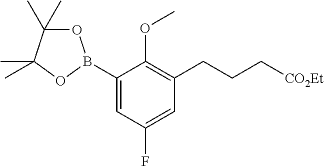 [SMILES: CCOC(=O)CCCc1cc(F)cc(B2OC(C)(C)C(C)(C)O2)c1OC]

To a solution of ethyl 4-[3-bromo-5-fluoro-2-(methyloxy)phenyl]butanoate (D26) (234 mg), 4,4,4′,4′,5,5,5′,5′-octamethyl-2,2′-bi-1,3,2-dioxaborolane (335 mg), tricyclohexylphosphine (103 mg) and potassium acetate (144 mg) in N,N-dimethylformamide (DMF) (2 mL) was added Pd₂ dba₃ (26.9 mg) under nitrogen. The reaction vessel was sealed and heated under microwave at 150° C. for 45 min. After cooling the reaction, the mixture was diluted with brine, extracted with ethyl acetate, the combined organic phases were dried over anhydrous sodium sulphate and concentrated, the residue was purified by column chromatography to give ethyl 4-[5-fluoro-2-(methyloxy)-3-(4,4,5,5-tetramethyl-1,3,2-dioxaborolan-2-yl)phenyl]butanoate (D27) (113 mg). MS (ES): C₁₉H₂₈BFO₅ requires 366; found 367.3 (M+H⁺).

### Description for D28

Ethyl 4-[3-(2-chloro-5-pyrimidinyl)-5-fluoro-2-(methyloxy)phenyl]butanoate (D28)

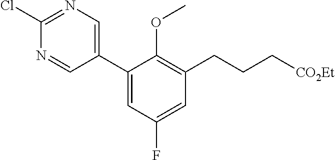 [SMILES: CCOC(=O)CCCc1cc(F)cc(-c2cnc(Cl)nc2)c1OC]

To a mixture of ethyl 4-[5-fluoro-2-(methyloxy)-3-(4,4,5,5-tetramethyl-1,3,2-dioxaborolan-2-yl)phenyl]butanoate (D27) (200 mg), 5-bromo-2-chloropyrimidine (169 mg) and tripotassium phosphate (290 mg) in 1,2-dimethoxyethane (DME) (10 mL) and water (2 mL) under nitrogen was added Pd(Ph₃P)₄ (63.1 mg). The reaction vessel was sealed and heated under microwave at 120° C. for 15 min. After cooling the reaction, the mixture was concentrated and the residue was purified by column chromatography to give ethyl 4-[3-(2-chloro-5-pyrimidinyl)-5-fluoro-2-(methyloxy)phenyl]butanoate (D28) (80 mg) as a brown oil. MS (ES): C₁₇H₁₈ClFN₂O₃ requires 352; found 353.2 (M+H⁺).

### Description for D29

Ethyl 4-[5-fluoro-3-{2-[4-[(1-methylethyl)oxy]-3-(trifluoromethyl)phenyl]-5-pyrimidinyl}-2-(methyloxy)phenyl]butanoate (D29)

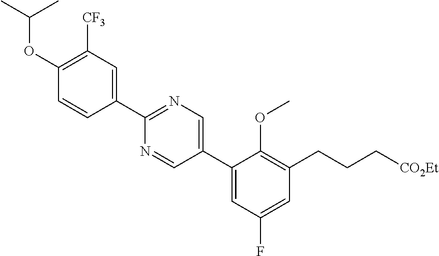 [SMILES: CCOC(=O)CCCc1cc(F)cc(-c2cnc(-c3ccc(OC(C)C)c(C(F)(F)F)c3)nc2)c1OC]

To a mixture of 4,4,5,5-tetramethyl-2-[4-[(1-methylethyl)oxy]-3-(trifluoromethyl)phenyl]-1,3,2-dioxaborolane (44.9 mg) (D14), ethyl 4-[3-(2-chloro-5-pyrimidinyl)-5-fluoro-2-(methyloxy)phenyl]butanoate (D28) (40 mg) and tripotassium phosphate (48.1 mg) in 1,2-dimethoxyethane (DME) (10 mL) and water (2 mL) under nitrogen was added Pd(Ph₃P)₄ (13.10 mg). The reaction vessel was sealed and heated under microwave at 120° C. for 15 min. After cooling the reaction, the mixture was concentrated and the residue was purified by column chromatography to give ethyl 4-[5-fluoro-3-{2-[4-[(1-methylethyl)oxy]-3-(trifluoromethyl)phenyl]-5-pyrimidinyl}-2-(methyloxy)phenyl]butanoate (D29) (40 mg) as a brown oil. MS (ES): C₂₇H₂₈F₄N₂O₄ requires 520; found 521.1 (M+H⁺).

### Description for D30

Ethyl 4-[3-(2-{3-cyano-4-[(1-methylethyl)oxy]phenyl}-5-pyrimidinyl)-5-fluoro-2-(methyloxy)phenyl]butanoate (D30)

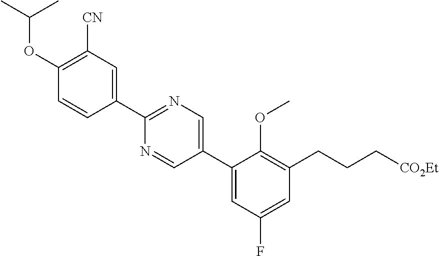 [SMILES: CCOC(=O)CCCc1cc(F)cc(-c2cnc(-c3ccc(OC(C)C)c(C#N)c3)nc2)c1OC]

To a mixture of 2-[(1-methylethyl)oxy]-5-(4,4,5,5-tetramethyl-1,3,2-dioxaborolan-2-yl)benzonitrile (D9) (39.1 mg), ethyl 4-[3-(2-chloro-5-pyrimidinyl)-5-fluoro-2-(methyloxy)phenyl]butanoate (D28) (40 mg) and tripotassium phosphate (48.1 mg) in 1,2-dimethoxyethane (DME) (10 mL) and water (2 mL) under nitrogen was added Pd(Ph₃P)₄ (13.10 mg). The reaction vessel was sealed and heated under microwave at 120° C. for 15 min. After cooling the reaction, the mixture was concentrated and the residue was purified by column chromatography to give ethyl 4-[3-(2-{3-cyano-4-[(1-methylethyl)oxy]phenyl}-5-pyrimidinyl)-5-fluoro-2-(methyloxy)phenyl]butanoate (D30) (50 mg) as a brown oil. MS (ES): C₂₇H₂₈FN₃O₄ requires 477; found 478.2 (M+H⁺).

### Description for D31

2-bromo-6-nitrophenyl methyl ether (D31)

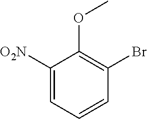

To a mixture of K₂CO₃ (2.54 g) and 2-bromo-6-nitrophenol (2 g) in acetone (15 mL) at room temperature was added Mel (3.442 mL). The reaction mixture was heated to 80° C. for 6 h, after cooling the reaction, the mixture was filtered and the filtrate was concentrated to afford 2-bromo-6-nitrophenyl methyl ether (D31) (2.1 g). δH (CDCl₃, 400 MHz): 3.96 (3H, s), 4.91 (1H, m), 7.06 (1H, t), 7.19 (1H, s), 7.72 (1H, m).

### Description for D32

Ethyl 4-[2-(methyloxy)-3-nitrophenyl]butanoate (D32)

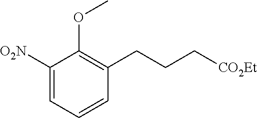 [SMILES: CCOC(=O)CCCc1cccc([N+](=O)[O-])c1OC]

To a mixture of 2-bromo-6-nitrophenyl methyl ether (D31) (1.02 g), Pd2(dba)₃ (0.604 g), tri-t-butylphosphine (0.381 g) and cesium carbonate (0.573 g) in tetrahydrofuran (THF) (40 mL) was added bromo[4-(ethyloxy)-4-oxobutyl]zinc (17.58 mL) under nitrogen. The reaction mixture was stirred at room temperature overnight. The reaction was quenched with saturated NH₄Cl solution. The organic phase was separated and the aqueous phase was extracted with EtOAc. The combined organic solution was dried over anhydrous sodium sulphate. After filtration and concentration, the residue was purified by column chromatography to give ethyl 4-[2-(methyloxy)-3-nitrophenyl]butanoate (D32) (1.2 g). MS (ES): C₁₃H₁₇NO₅ requires 267; found 268.1 (M+H⁺).

### Description for D33

Ethyl 4-[3-amino-2-(methyloxy)phenyl]butanoate (D33)

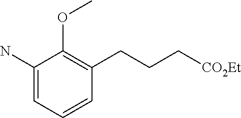 [SMILES: CCOC(=O)CCCc1cccc(N)c1OC]

To a solution of ethyl 4-[2-(methyloxy)-3-nitrophenyl]butanoate (D32) (1.2 g) in ethanol (40 mL) was added Raney nickle (0.063 g) under nitrogen. The reaction mixture was stirred under hydrogen at room temperature overnight. The mixture was filtered and the filtrate was concentrated and the residue was purified by column chromatography to give ethyl 4-[3-amino-2-(methyloxy)phenyl]butanoate (D33) (0.96 g). MS (ES): C₁₃H₁₉NO₃ requires 237; found 238.2 (M+H⁺).

### Description for D34

Ethyl 4-[3-bromo-2-(methyloxy)phenyl]butanoate (D34)

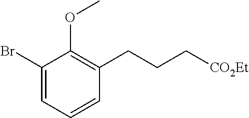 [SMILES: CCOC(=O)CCCc1cccc(Br)c1OC]

To a cold solution of HBr (3 mL,) was added ethyl 4-[3-amino-2-(methyloxy)phenyl]butanoate (D33) (0.7 g) to form a suspension of HBr salt at 0˜5° C.

The cold suspension was added to a cold solution of sodium nitrite (0.214 g) in water (2 mL) at 0˜5° C. The suspension was stirred at this temperature for 10 min and then added to a hot mixture of copper(I) bromide (0.423 g) in HBr (1.8 mL) at 80° C. The reaction mixture was stirred at 80° C. for 10 min. After cooling the reaction, the mixture was neutralized with solid sodium bicarbonate. The reaction mixture was filtered and the filtrate was partitioned between ethyl acetate (300 mL) and saturated brine (50 mL). The organic phase was dried over sodium sulphate and concentrated to give the mixture of ethyl 4-[3-bromo-2-(methyloxy)phenyl]butanoate (D34) and 4-[3-bromo-2-(methyloxy)phenyl]butanoic acid (0.51 g). MS (ES): C₁₃H₁₇BrO₃ requires 300; found 301.1 (M+H⁺).

### Description for D35

Ethyl 4-[2-(methyloxy)-3-(4,4,5,5-tetramethyl-1,3,2-dioxaborolan-2-yl)phenyl]butanoate (D35)

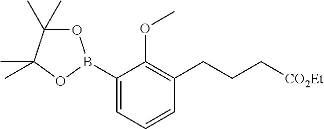 [SMILES: CCOC(=O)CCCc1cccc(B2OC(C)(C)C(C)(C)O2)c1OC]

To a suspension of ethyl 4-[3-bromo-2-(methyloxy)phenyl]butanoate (D34) (230 mg), 4,4,4′,4′,5,5,5′,5′-octamethyl-2,2′-bi-1,3,2-dioxaborolane (291 mg) and potassium acetate (187 mg) in N,N-dimethylformamide (DMF) (30 mL) stirred under nitrogen at room temperature was added PdCl₂(dppf) (55.9 mg). The reaction mixture was stirred at 90° C. for 16 h. After cooling the reaction, the mixture was concentrated, the residue was diluted with ethyl acetate (50 mL) and filtered through Celite. The filtrate was washed with water and separated. The organic phase was dried over anhydrous sodium sulfate. After removing the solvent, the crude product was purified by column chromatography to give ethyl 4-[2-(methyloxy)-3-(4,4,5,5-tetramethyl-1,3,2-dioxaborolan-2-yl)phenyl]butanoate (D35) (170 mg). MS (ES): C₁₉H₂₉BO₅ requires 348; found 349.2 (M+H⁺).

### Description for D36

Ethyl 4-[3-(2-chloro-5-pyrimidinyl)-2-(methyloxy)phenyl]butanoate (D36)

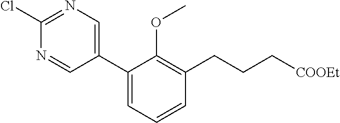 [SMILES: CCOC(=O)CCCc1cccc(-c2cnc(Cl)nc2)c1OC]

To a solution of 5-bromo-2-chloropyrimidine (222 mg), ethyl 4-[2-(methyloxy)-3-(4,4,5,5-tetramethyl-1,3,2-dioxaborolan-2-yl)phenyl]butanoate (D35) (200 mg) and tripotassium phosphate (305 mg) in 1,2-dimethoxyethane (DME) (5 mL) and water (1.250 mL) stirred under nitrogen at room temperature was added Pd(Ph₃P)₄ (66.4 mg) in one charge. The reaction vessel was sealed and heated under microwave at 130° C. for 15 min. After cooling the reaction, the reaction mixture was filtered and the filtrate partitioned between ethyl acetate (250 mL) and saturated brine (50 mL). The organic phase was dried over sodium sulphate and concentrated, the residue was purified by column chromatography to give ethyl 4-[3-(2-chloro-5-pyrimidinyl)-2-(methyloxy)phenyl]butanoate (D36) (162 mg). MS (ES): C₁₇H₁₉ClN₂O₃ requires 334; found 335.1 (M+H⁻¹).

### Description for D37

Ethyl 4-[3-(2-{3-cyano-4-[(1-methylethyl)oxy]phenyl}-5-pyrimidinyl)-2-(methyloxy)phenyl]butanoate (D37)

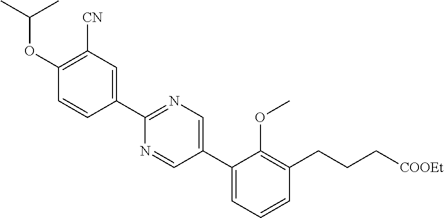 [SMILES: CCOC(=O)CCCc1cccc(-c2cnc(-c3ccc(OC(C)C)c(C#N)c3)nc2)c1OC]

To a solution of 2-[(1-methylethyl)oxy]-5-(4,4,5,5-tetramethyl-1,3,2-dioxaborolan-2-yl)benzonitrile (D9) (139 mg), ethyl 4-[3-(2-chloro-5-pyrimidinyl)-2-(methyloxy)phenyl]butanoate (D36) (162 mg) and tripotassium phosphate (257 mg) in 1,2-dimethoxyethane (DME) (3 mL) and water (0.750 mL) stirred under nitrogen at room temperature was added Pd(Ph₃P)₄ (55.9 mg) in one charge. The reaction vessel was sealed and heated under microwave at 130° C. for 15 min. After cooling the reaction, the reaction mixture was filtered and the filtrate was partitioned between ethyl acetate (70 mL) and saturated brine (20 mL). The organic phase was dried over sodium sulphate and concentrated to give ethyl 4-[3-(2-{3-cyano-4-[(1-methylethyl)oxy]phenyl}-5-pyrimidinyl)-2-(methyloxy)phenyl]butanoate (D37) (120 mg). MS (ES): C₂₇H₂₉N₃O₄ requires 459; found 460.1 (M+H⁺).

### Description for D38

3-(2-chloro-5-pyrimidinyl)-2-ethylbenzaldehyde (D38)

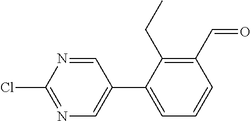 [SMILES: CCc1c(C=O)cccc1-c1cnc(Cl)nc1]

To a mixture of 2-ethyl-3-(4,4,5,5-tetramethyl-1,3,2-dioxaborolan-2-yl)benzaldehyde (700 mg), 5-bromo-2-chloropyrimidine (531 mg) and tripotassium phosphate (1428 mg) in 1,2-dimethoxyethane (DME) (10 mL) and water (2 mL) under nitrogen was added Pd(Ph₃P)₄ (311 mg). The mixture stirred at 120° C. for 2 h. The solvent was removed in vacuo, the residue was purified by column chromatography to give 3-(2-chloro-5-pyrimidinyl)-2-ethylbenzaldehyde (D38) (680 mg) as a brown oil. MS (ES): C₁₃H₁₁ClN₂O requires 246; found 247.1 (M+H⁺).

### Description for D39

2-ethyl-3-{2-[4-[(1-methylethyl)oxy]-3-(trifluoromethyl)phenyl]-5-pyrimidinyl}Benzaldehyde (D39)

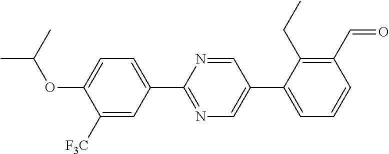 [SMILES: CCc1c(C=O)cccc1-c1cnc(-c2ccc(OC(C)C)c(C(F)(F)F)c2)nc1]

To a mixture of 3-(2-chloro-5-pyrimidinyl)-2-ethylbenzaldehyde (D38) (680 mg), 4,4,5,5-tetramethyl-2-[4-[(1-methylethyl)oxy]-3-(trifluoromethyl)phenyl]-1,3,2-dioxaborolane (D14) (1001 mg) and tripotassium phosphate (1170 mg) in N,N-dimethylformamide (DMF) (10 mL) and water (2 mL) under nitrogen was added Pd(Ph₃P)₄ (319 mg). The reaction mixture was sealed and heated under microwave at 120° C. for 15 min. The solvent was removed in vacuo, the residue was purified by column chromatography to give 2-ethyl-3-{2-[4-[(1-methylethyl)oxy]-3-(trifluoromethyl)phenyl]-5-pyrimidinyl}benzaldehyde (D39) (800 mg) as a brown oil. MS (ES): C₂₃H₂₁F₃N₂O₂ requires 414; found 415.2 (M+H⁺).

### Description for D40

ethyl N-[(2-ethyl-3-{2-[4-[(1-methylethyl)oxy]-3-(trifluoromethyl)phenyl]-5-pyrimidinyl}phenyl)methyl]-b-alaninate (D40)

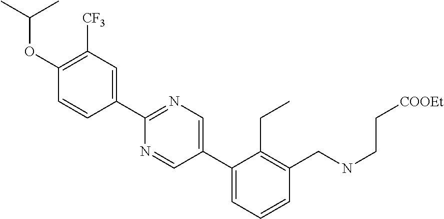 [SMILES: CCOC(=O)CCNCc1cccc(-c2cnc(-c3ccc(OC(C)C)c(C(F)(F)F)c3)nc2)c1CC]

To a mixture of 2-ethyl-3-{2-[4-[(1-methylethyl)oxy]-3-(trifluoromethyl)phenyl]-5-pyrimidinyl}benzaldehyde (D39) (50 mg) and ethyl β-alaninate (70.7 mg) in ethanol (10.00 mL) at room temperature was added sodium acetate (49.5 mg) and acetic acid (36.2 mg). After stirring for 20 min, the solvent was removed in vacuo and dichloromethane (DCM) (10 mL) was added into the residue followed by sodium triacetoxyborohydride (77 mg). The mixture was stirred at room temperature overnight. Sat. NaHCO₃ (5 mL) was added into the reaction mixture and the resulting solution was stirred for 5 min. The mixture was partitioned between brine (20 mL) and EtOAc (100 mL). The organic phase was concentrated and dried over sodium sulphate to afford the crude product ethyl N-[(2-ethyl-3-{2-[4-[(1-methylethyl)oxy]-3-(trifluoromethyl)phenyl]-5-pyrimidinyl}phenyl)methyl]-b-alaninate (D40) (50 mg). MS (ES): C₂₈H₃₂F₃N₃O₃ requires 515; found 516.1 (M+H⁺).

### Description for D41

ethyl 1-[(2-ethyl-3-{2-[4-[(1-methylethyl)oxy]-3-(trifluoromethyl)phenyl]-5-pyrimidinyl}phenyl)methyl]-4-piperidinecarboxylate (D41)

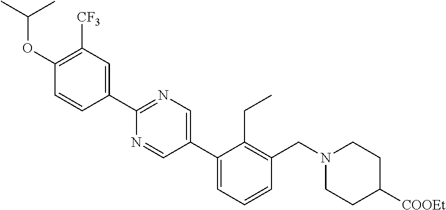 [SMILES: CCOC(=O)C1CCN(Cc2cccc(-c3cnc(-c4ccc(OC(C)C)c(C(F)(F)F)c4)nc3)c2CC)CC1]

To a mixture of 2-ethyl-3-{2-[4-[(1-methylethyl)oxy]-3-(trifluoromethyl)phenyl]-5-pyrimidinyl}benzaldehyde (D39) (80 mg) and ethyl 4-piperidinecarboxylate (182 mg) in ethanol (20.00 mL) at room temperature was added acetic acid (11.59 mg). After stirring for 1 h, the solvent was removed in vacuo and dichloromethane (DCM) (20 mL) was added into the residue followed by sodium triacetoxyborohydride (123 mg). The mixture was stirred at room temperature overnight. Sat. NaHCO₃ (5 mL) was added into the reaction mixture and the resulting solution was stirred for 5 min. The mixture was partitioned between brine (20 mL) and EtOAc (100 mL). The organic phase was concentrated and dried over sodium sulphate to afford the crude product ethyl 1-[(2-ethyl-3-{2-[4-[(1-methylethyl)oxy]-3-(trifluoromethyl)phenyl]-5-pyrimidinyl}phenyl)methyl]-4-piperidinecarboxylate (D41) (70 mg). MS (ES): C₃₁H₃₆F₃N₃O₃ requires 555; found 556.3 (M+H⁺).

### Description for D42

2-(2-methylpropyl)-5-nitrobenzonitrile (D42)

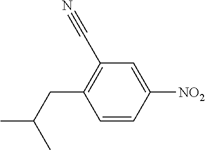

To a solution of isobutylboronic acid (5.89 g), 2-bromo-5-nitrobenzo nitrile (12.5 g) and Cs₂CO₃ (35.9 g) in toluene (150 mL) and water (5 mL) stirred under nitrogen at room temperature was added solid PdCl₂(dppf)-CH₂Cl₂ adduct (2.248 g) in one charge. The reaction mixture was stirred at 100° C. for 16 h. After cooling the reaction, the solvent was removed in vacuo. The residue was purified by column chromatography to give 2-(2-methylpropyl)-5-nitrobenzonitrile (D42) (11 g) as a light yellow oil. δH (CDCl₃, 400 MHz): 1.00 (6H, d), 2.06 (1H, m), 2.86 (2H, d), 7.52 (1H, d), 8.37 (1H, dd), 8.51 (1H, d).

### Description for D43

5-amino-2-(2-methylpropyl)benzonitrile (D43)

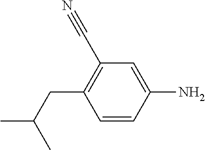

To a solution of 2-isobutyl-5-nitrobenzonitrile (D42) (7.5 g) in methanol (80 mL) and water (80 mL) was added ammonium formate (51.9 g) and zinc (26.9 g). The reaction mixture was stirred at 80° C. for 4 h. After cooling the reaction, the solid was filtered, the filtrate was concentrated under reduced pressure, the residue was extracted with EtOAc (300 mL*2), the combined organic layers were washed with water (30 mL*2), dried and concentrated to give 5-amino-2-(2-methylpropyl)benzonitrile (D43) (17.5 g) as a colorless solid. MS (ES): C₁₁H₁₄N₂ requires 174; found 175.1 (M+H⁺).

### Description for D44

5-bromo-2-(2-methylpropyl)benzonitrile (D44)

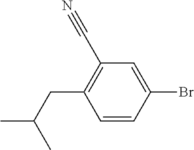

To a solution of 5-amino-2-isobutylbenzonitrile (D43) (34 g) in acetonitrile (500 mL) was added HBr (24.37 mL) at 0° C. Then a solution of sodium nitrite (16.16 g) in water (50 mL) was added to the reaction mixture. After stirring for 30 min, copper(II) bromide (87 g) and copper(I) bromide (5.60 g) were added to the reaction mixture. The mixture was stirred at room temperature for 16 h. The reaction was quenched with saturated aqueous sodium bicarbonate solution. The mixture was extracted with EA. The combined organic solution was dried over anhydrous sodium sulphate. After filtration and concentration, the residue was purified by column chromatography to give 5-bromo-2-(2-methylpropyl)benzonitrile (D44) (36 g) as a colorless oil. δH (CDCl₃, 400 MHz): 0.96 (6H, d), 1.98 (1H, m), 2.69 (2H, d), 7.18 (1H, d), 7.64 (1H, dd), 7.55 (1H, d).

### Description for D45

2-(2-methylpropyl)-5-(4,4,5,5-tetramethyl-1,3,2-dioxaborolan-2-yl)benzonitrile (D45)

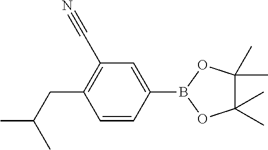 [SMILES: CC(C)Cc1ccc(B2OC(C)(C)C(C)(C)O2)cc1C#N]

To a solution of 5-bromo-2-isobutylbenzonitrile (D44) (10 g, 42.0 mmol), 4,4,4′,4′,5,5,5′,5′-octamethyl-2,2′-bi(1,3,2-dioxaborolane) (11.20 g) and potassium acetate (12.36 g) in 1,4-dioxane (150 mL) stirred at room temperature under nitrogen was added PdCl₂(dppf)-CH₂Cl₂ adduct (1.715 g). The reaction mixture was heated and stirred at 80° C. for 12 h. The solvent was removed under reduced pressure. The residue was dissolved in EA (300 mL) and the organic phase was washed with water and brine, dried over anhydrous Na₂SO₄. The dried solution was concentrated and the residue was purified by column chromatography to give 2-(2-methylpropyl)-5-(4,4,5,5-tetramethyl-1,3,2-dioxaborolan-2-yl)benzonitrile (D45) (10.1 g) as a white solid. δH (CDCl₃, 400 MHz): 0.96 (6H, d), 1.37 (12H, s), 2.01 (1H, m), 2.74 (2H, d), 7.29 (1H, d), 7.90 (1H, d), 8.07 (1H, s).

### Description for D46

ethyl 4-(3-{2-[3-cyano-4-(2-methylpropyl)phenyl]-5-pyrimidinyl}-2-ethylphenyl) butanoate (D46)

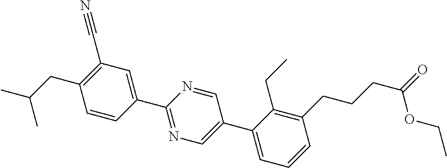 [SMILES: CCOC(=O)CCCc1cccc(-c2cnc(-c3ccc(CC(C)C)c(C#N)c3)nc2)c1CC]

To a solution of 2-(2-methylpropyl)-5-(4,4,5,5-tetramethyl-1,3,2-dioxaborolan-2-yl)benzonitrile (D45) (76 mg, 0.267 mmol), ethyl 4-[3-(2-chloro-5-pyrimidinyl)-2-ethylphenyl]butanoate (D7) (89 mg, 0.267 mmol) and tripotassium phosphate (142 mg, 0.669 mmol) in 1,2-Dimethoxyethane (DME) (4 mL) and Water (1.000 mL) stirred under nitrogen at room temperature was added Pd(Ph₃P)₄ (15.45 mg, 0.013 mmol) in one charge. The reaction vessel was sealed and heated in Biotage Initiator using initial high to 120° C. for 40 min. After cooling the reaction, the reaction mixture was partitioned between ethyl acetate (250 mL) and saturated brine (50 mL). The organic phase was dried over sodium sulphate and evaporated in vacuo to give the crude product. The crude product was purified by column chromatography to afford ethyl 4-(3-{2-[3-cyano-4-(2-methylpropyl)phenyl}-5-pyrimidinyl}-2-ethylphenyl) butanoate (D46) (75 mg). MS (ES): C₂₉H₃₃N₃O₂ requires 455.2; found 456.1 (M+H⁺).

### Description for D47

5-bromo-2-(3-chloro-4-isopropoxy-phenyl)-pyrimidine (D47)

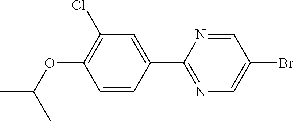 [SMILES: CC(C)Oc1ccc(-c2ncc(Br)cn2)cc1Cl]

A mixture of 2-(3-chloro-4-isopropoxy-phenyl)-4,4,5,5-tetramethyl-[1,3,2]dioxaborolane (D11) (10 g, 33.7 mmol), 5-bromo-2-iodo-pyrimidine (14.25 g, 50.0 mmol), Pd(dppf)Cl₂ (1.37 g, 1.68 mmol) and Na₂CO₃ (10.6 g, 100.0 mmol) in DME/H₂O (100 mL, 1:1) was refluxed overnight under nitrogen. Then the reaction mixture was cooled to room temperature, extracted with DCM, dried over sodium sulphate and concentrated in vacuo. The residue was purified by column chromatography to afford 5-bromo-2-(3-chloro-4-isopropoxy-phenyl)-pyrimidine (D47) (5.8 g). δH (DMSO-d₅, 400 MHz): 8.98 (2H, s), 8.26 (1H, s), 8.21 (1H, d), 7.27 (1H, d), 4.76 (1H, m), 1.28 (6H, d).

### Description for D48

1-benzyl-4-(3-bromo-2-ethyl-phenyl)-piperidin-4-ol (D48)

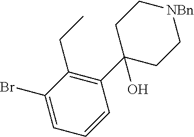 [SMILES: CCc1c(Br)cccc1C1(O)CCN(Cc2ccccc2)CC1]

To a solution of 1,3-dibromo-2-ethyl-benzene (30 g, 113.65 mmol) in THF (150 mL) was added 45.5 mL of BuLi (2.5M in hexane, 113.65 mmol) under nitrogen. The mixture was stirred for 2 hr at −78° C. Then N-benzyl-4-piperidone (21.51 g, 113.65 mmol) was added to the above mixture and stirred overnight. The mixture was poured into sat. aqueous NH₄Cl solution and extracted with DCM. The organic layer was separated, dried over Na₂SO₄, and concentrated in vacuo to afford 1-benzyl-4-(3-bromo-2-ethyl-phenyl)-piperidin-4-ol (D48) (30.4 g). MS (ES): C₂₀H₂₄BrNO requires 373; found 374 (M+H⁺).

### Description for D49

1-benzyl-4-(3-bromo-2-ethyl-phenyl)-1,2,3,6-tetrahydro-pyridine (D49)

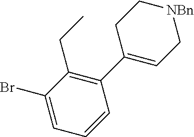 [SMILES: CCc1c(Br)cccc1C1=CCN(Cc2ccccc2)CC1]

To a solution of 1-benzyl-4-(3-bromo-2-ethyl-phenyl)-piperidin-4-ol (D116) (30 g, 80.15 mmol) in HOAc (300 mL) was added hydrochloric acid (45 mL, 37%). The reaction mixture was refluxed for 12 hr and concentrated in vacuo. The residue was purified by column chromatography to afford 1-benzyl-4-(3-bromo-2-ethyl-phenyl)-1,2,3,6-tetrahydro-pyridine (D49) (24 g). MS (ES): C₂₀H₂₂BrN requires 355; found 356 (M+H⁺).

### Description for D50

1-Benzyl-4-(2-ethyl-3-(4,4,5,5-tetramethyl-[1,3,2]dioxaborolan-2-yl)-phenyl]-1,2,3,6-tetrahydro-pyridine (D50)

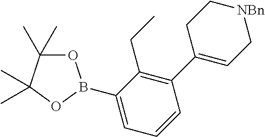 [SMILES: CCc1c(B2OC(C)(C)C(C)(C)O2)cccc1C1=CCN(Cc2ccccc2)CC1]

A mixture of 1-benzyl-4-(3-bromo-2-ethyl-phenyl)-1,2,3,6-tetrahydro-pyridine (D49) (20 g, 56.13 mmol), 4,4,4′,4′,5,5,5′,5′-octamethyl-2,2′-bi-1,3,2-dioxaborolane (17.46 g, 68.76 mmol) and potassium acetate (16.52 g, 168.40 mmol), PdCl2(dppf)-CH₂Cl₂ adduct (4.58 g, 5.613 mmol) in dioxane (300 mL) was degassed with nitrogen, followed by bubbling nitrogen gas through the stirred reaction mixture for 5 minutes. The reaction mixture was heated to 90° C. overnight. The reaction mixture was allowed to cool to room temperature and dioxane removed in vacuo. The residue was partitioned between ethyl acetate and water. The mixture was filtered through celite, washed with ethyl acetate. The combined organic phases were concentrated and purified by column chromatography to afford 1-benzyl-4-[2-ethyl-3-(4,4,5,5-tetramethyl-[1,3,2]dioxaborolan-2-yl)-phenyl]-1,2,3,6-tetrahydro-pyridine (D50) (15 g). MS (ES): C₂₆H₃₄BNO₂ requires 403; found 404 (M+H⁺)

### Description for D51

4-[2-ethyl-3-(4,4,5,5-tetramethyl-1,3,2-dioxaborolan-2-yl)phenyl]piperidine (D51)

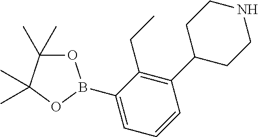 [SMILES: CCc1c(B2OC(C)(C)C(C)(C)O2)cccc1C1CCNCC1]

To a solution of 1-benzyl-4-[2-ethyl-3-(4,4,5,5-tetramethyl-[1,3,2]dioxaborolan-2-yl)-phenyl]-1,2,3,6-tetrahydro-pyridine (D50) (8 g, 19.83 mmol) in EtOAc (300 mL) was added dry Pd/C (8 g, 10%). The reaction mixture was hydrogenated under 55 psi at 50° C. for 12 hr, and then filtered. The filtrate was concentrated in vacuo. The residue was purified by Mass Directed AutoPrep to give 4-[2-Ethyl-3-(4,4,5,5-tetramethyl-[1,3,2]dioxaborolan-2-yl)-phenyl]-piperidine (D51) (3.5 g). MS (ES): C₁₉H₃₀BNO₂ requires 315.2; found 316.2 (M−H⁺)

### Description for D52

2-(3-chloro-4-[(1-methylethyl)oxy]phenyl}-5-[2-ethyl-3-(4-piperidinyl)phenyl]pyrimidine (D52)

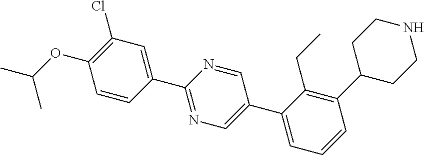 [SMILES: CCc1c(-c2cnc(-c3ccc(OC(C)C)c(Cl)c3)nc2)cccc1C1CCNCC1]

To a solution of 4-[2-Ethyl-3-(4,4,5,5-tetramethyl-[1,3,2]dioxaborolan-2-yl)-phenyl]-piperidine (D51) (346 mg, 0.806 mmol), 5-bromo-2-(3-chloro-4-isopropoxy-phenyl)-pyrimidine (D47) (240 mg, 0.733 mmol) and cesium carbonate (597 mg, 1.831 mmol) in 1,2-Dimethoxyethane (DME) (10 mL) and Water (2.500 mL) stirred under nitrogen at room temp° C. was added Pd(Ph3P)₄ (42.3 mg, 0.037 mmol) in one charge. The reaction vessel was sealed and heated in Biotage Initiator using initial high to 120° C. for 30 min. After cooling the reaction, the reaction mixture was filtered and the filtrate was partitioned between ethyl acetate 250 mL and saturated brine 50 mL. The organic phase was dried over sodium sulphate and evaporated in vacuo to afford 2-{3-chloro-4-[(1-methylethyl)oxy]phenyl}-5-[2-ethyl-3-(4-piperidinyl)phenyl]pyrimidine (D52) (0.5 g), which was used for next step without further purification.

### Description for D53

ethyl 3-{4-[3-(2-{3-chloro-4-[(1-methylethyl)oxy]phenyl}-5-pyrimidinyl)-2-ethylphenyl]-1-piperidinyl}propanoate (D53)

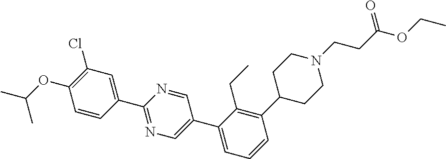 [SMILES: CCOC(=O)CCN1CCC(c2cccc(-c3cnc(-c4ccc(OC(C)C)c(Cl)c4)nc3)c2CC)CC1]

To a solution of 2-{3-chloro-4-[(1-methylethyl)oxy]phenyl}-5-[2-ethyl-3-(4-piperidinyl)phenyl]pyrimidine (D52) (0.25 g, 0.350 mmol) and ethyl 2-propenoate (0.140 g, 1.399 mmol) in Acetonitrile (10 mL) stirred under nitrogen at room temp° C. was added DBU (0.026 mL, 0.175 mmol) in one charge. The reaction vessel was sealed and heated to 80° C. for 3 h. After cooling the reaction, the reaction mixture was partitioned between ethyl acetate 250 mL and saturated brine 50 mL. The organic phase was dried over sodium sulphate and evaporated in vacuo to give the crude product. The crude product was purified by column chromatography to afford ethyl 3-{4-[3-(2-{3-chloro-4-[(1-methylethyl)oxy]phenyl}-5-pyrimidinyl)-2-ethylphenyl]-1-piperidinyl}propanoate (D53) (120 mg). MS (ES): C₃₁H₃₈ClN₃O₃ requires 535.2; found 536.1 (M+H⁺).

### Description for D54

ethyl 4-{4-[3-(2-{3-chloro-4-[(1-methylethyl)oxy]phenyl}-5-pyrimidinyl)-2-ethylphenyl]-1-piperidinyl}butanoate (D54)

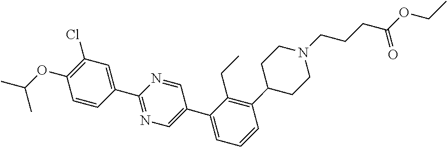 [SMILES: CCOC(=O)CCCN1CCC(c2cccc(-c3cnc(-c4ccc(OC(C)C)c(Cl)c4)nc3)c2CC)CC1]

To a solution of 2-{3-chloro-4-[(1-methylethyl)oxy]phenyl}-5-[2-ethyl-3-(4-piperidinyl)phenyl]pyrimidine (D52) (0.25 g, 0.350 mmol) and potassium carbonate (0.145 g, 1.049 mmol) in Acetonitrile (10 mL) stirred under nitrogen at room temp° C. was added ethyl 4-bromobutanoate (0.273 g, 1.399 mmol) in one charge. The reaction vessel was sealed and heated to 80° C. for 3 h. After cooling the reaction, the reaction mixture was partitioned between ethyl acetate and saturated brine. The organic phase was dried over sodium sulphate and evaporated in vacuo to give the crude product. The crude product was purified by column chromatography to afford ethyl 4-{4-[3-(2-{3-chloro-4-[(1-methylethyl)oxy]phenyl}-5-pyrimidinyl)-2-ethylphenyl]-1-piperidinyl}butanoate (D54) (130 mg). MS (ES): C₃₂H₄₀ClN₃O₃ requires 549.2; found 550.1 (M+H⁺)

### Description for D55

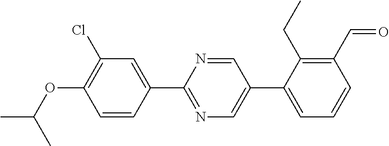 [SMILES: CCc1c(C=O)cccc1-c1cnc(-c2ccc(OC(C)C)c(Cl)c2)nc1]

To a solution of {3-chloro-4-[(1-methylethyl)oxy]phenyl}boronic acid (1.865 g, 8.70 mmol), 3-(2-chloro-5-pyrimidinyl)-2-ethylbenzaldehyde (D38) (1.43 g, 5.80 mmol) and cesium carbonate (4.72 g, 14.49 mmol) in 1,2-Dimethoxyethane (DME) (15 mL) and Water (3.75 mL) stirred under nitrogen at room temperature was added Pd(Ph₃P)₄ (0.335 g, 0.290 mmol) in one charge. The reaction vessel was sealed and heated in Biotage Initiator using initial high to 120° C. for 15 min. After cooling the reaction, the reaction mixture was filtered and the filtrate was partitioned between ethyl acetate and saturated brine. The organic phase was dried over sodium sulphate and evaporated in vacuo to afford 3-(2-{3-chloro-4-[(1-methylethyl)oxy]phenyl}-5-pyrimidinyl)-2-ethylbenzaldehyde (D55) (2.3 g), which was used for next step without further purification. δH (CDCl₃-d, 400 MHz): 1.17 (3H, t), 1.46 (6H, d), 3.03 (2H, q), 4.72 (1H, dt), 7.07 (1H, d), 7.48 (2H, mj, 7.99 (1H, dd), 8.37 (1H, dd), 8.57 (1H, d), 8.74 (2H, s), 10.40 (1H, s).

### Description for D56

5-[5-(2-ethyl-3-formylphenyl)-2-pyrimidinyl]-2-(2-methylpropyl)benzonitrile (D56)

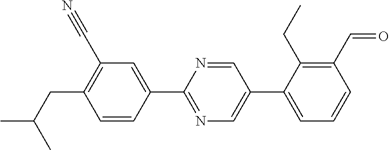 [SMILES: CCc1c(C=O)cccc1-c1cnc(-c2ccc(CC(C)C)c(C#N)c2)nc1]

To a solution of 3-(2-chloro-5-pyrimidinyl)-2-ethylbenzaldehyde (D38) (0.865 g, 3.51 mmol), 2-(2-methylpropyl)-5-(4,4,5,5-tetramethyl-1,3,2-dioxaborolan-2-yl)benzonitrile (D45) (1 g, 3.51 mmol) and tripotassium phosphate (1.861 g, 8.77 mmol) in 1,2-Dimethoxyethane (DME) (16 mL) and Water (4.00 mL) stirred under nitrogen at room temp° C. was added Pd(Ph₃P)₄ (0.405 g, 0.351 mmol) in one charge. The reaction vessel was sealed and heated in Biotage Initiator using initial high to 130° C. for 15 min. After cooling the reaction, the reaction mixture was filtered and the filtrate was partitioned between ethyl acetate (250 mL) and saturated brine (50 mL). The organic phase was dried over sodium sulphate and evaporated in vacuo to give the crude product. The crude product was purified by column chromatography to afford 5-[5-(2-ethyl-3-formylphenyl)-2-pyrimidinyl]-2-(2-methylpropyl)benzonitrile (D56) (1.25 g). MS (ES): C₂₄H₂₃N₃O requires 369.2; found 370.1 (M+H⁺).

### Description for D57

5-(5-bromo-pyrimidin-2-O-2-isopropoxy-benzonitrile (D57)

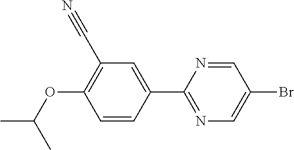 [SMILES: CC(C)Oc1ccc(-c2ncc(Br)cn2)cc1C#N]

A mixture of 2-isopropoxy-5-(4,4,5,5-tetramethyl-[1,3,2]dioxaborolan-2-yl) -benzonitrile (D9) (10 g, 34.8 mmol), 5-bromo-2-iodo-pyrimidine (11.9 g, 41.8 mmol), Pd(dppf)Cl₂ (1.42 g, 1.74 mmol) and Na₂CO₃(7.4 g, 69.6 mmol) in DME/H₂O (200 mL, 1:1) was stirred at reflux under nitrogen overnight. Then the mixture was cooled to room temperature, extracted with DCM, dried and concentrated. The residue was purified by column chromatography to afford 5-(5-bromo-pyrimidin-2-yl)-2-isopropoxy-benzonitrile (D57) (6 g). MS (ES): C₁₄H₁₂BrN₃O requires 317; found 318 (M+H⁺).

### Description for D58

5-[5-(2-ethyl-3-formylphenyl)-2-pyrimidinyl]-2-[(1-methylethyl)oxy]benzonitrile (D58)

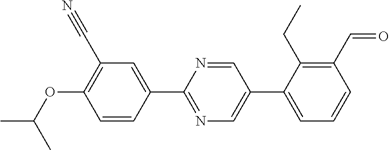 [SMILES: CCc1c(C=O)cccc1-c1cnc(-c2ccc(OC(C)C)c(C#N)c2)nc1]

To a solution of 5-(5-bromo-pyrimidin-2-yl)-2-isopropoxy-benzonitrile (D57) (489 mg, 1.538 mmol), 2-ethyl-3-(4,4,5,5-tetramethyl-1,3,2-dioxaborolan-2-yl)benzaldehyde (400 mg, 1.538 mmol) and tripotassium phosphate (816 mg, 3.84 mmol) in 1,2-Dimethoxyethane (DME) (10 mL) and Water (2.500 mL) stirred under nitrogen at room temp° C. was added Pd(Ph3P)₄ (178 mg, 0.154 mmol) in one charge. The reaction vessel was sealed and heated in Biotage Initiator using initial high to 120° C. for 1 h. After cooling the reaction, the reaction mixture was filtered and the filtrate was partitioned between ethyl acetate and saturated brine. The organic phase was dried over sodium sulphate and evaporated in vacuo to give the crude product. The crude product was purified by column chromatography to afford 5-[5-(2-ethyl-3-formylphenyl)-2-pyrimidinyl]-2-[(1-methylethyl)oxy]benzonitrile (D58) (269 mg). MS (ES): C₂₃H₂₁N₃O₂ requires 371.1; found 372.0 (M+H⁺).

### Description for D59

Methyl 1-{[3-(2-[3-cyano-4-(1-methylethyl)oxy]phenyl}-5-pyrimidinyl)-2-ethylphenyl]methyl]-3-azetidinecarboxylate (D59)

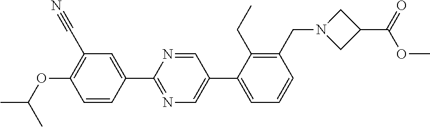 [SMILES: CCc1c(CN2CC(C(=O)OC)C2)cccc1-c1cnc(-c2ccc(OC(C)C)c(C#N)c2)nc1]

To a solution of 515-(2-ethyl-3-formylphenyl)-2-pyrimidinyl]-2-[(1-methylethyl)oxy]benzonitrile (D58) (249 mg, 0.670 mmol) and sodium acetate (220 mg, 2.68 mmol) in Dichloromethane (DCM) (10 mL) stirred under nitrogen at room temperature was added methyl 3-azetidinecarboxylate (254 mg, 1.676 mmol) in one charge. The reaction mixture was stirred at room temperature for 30 min. Then sodium triacetoxyborohydride (284 mg, 1.341 mmol) was added to the above mixture. The reaction mixture was stirred at room temperature for 2 h. The reaction mixture was partitioned between ethyl acetate and saturated brine. The organic phase was dried over sodium sulphate and evaporated in vacuo to afford methyl 1-{[3-(2-{3-cyano-4-[(1-methylethyl)oxy]phenyl}-5-pyrimidinyl)-2-ethylphenyl]methyl)-3-azetidinecarboxylate (D59) (315 mg), which was used for next step without further purification. MS (ES): C₂₈H₃₀N₄O₃ requires 470.2; found 471.1 (M+H⁺).

### Description for D60

5-(5-bromo-pyrimidin-2-yl)-2-isobutyl-benzonitrile (D60)

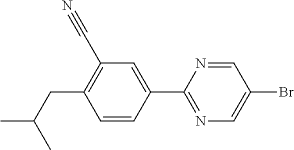 [SMILES: CC(C)Cc1ccc(-c2ncc(Br)cn2)cc1C#N]

To a solution of 2-isobutyl-5-(4,4,5,5-tetramethyl-[1,3,2]dioxaborolan-2-yl)-benzonitrile (D45) (6 g, 21 mmol) in DME/H₂O (60 mL/60 mL) was added 5-bromo-2-iodo-pyrimidine (7.2 g, 25 mmol), Na₂CO₃ (4.5 g, 42 mmol) and Pd(dppf)Cl₂ (1.5 g, 2.1 mmol). The mixture was degassed with nitrogen and stirred at 90° C. overnight. Then the mixture was cooled to room temperature and extracted with DCM. The combined organic layer was dried over Na₂SO₄, concentrated in vacuo and purified by column chromatography to afford 5-(5-bromo-pyrimidin-2-yl)-2-isobutyl-benzonitrile (D60) (5.6 g). δH (CDCl₃-d, 400 MHz): 8.83 (2H, s), 8.67 (1H, d,), 8.50 (1H, dd), 7.38 (1H, d), 2.77 (2H, d), 2.03 (1H, m), 0.96 (6H, d).

### Description for D61

2-[2-Ethyl-3-(2-methoxy-vinyl)-phenyl]-4,4,5,5-tetramethyl-[1,3,2]dioxa-borolane (D61)

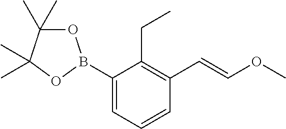

A mixture of 2-Ethyl-3-(4,4,5,5-tetramethyl-[1,3,2]dioxaborolan-2-yl)-benzaldehyde (100 g; 384.41 mmol), (Methoxymethyl)triphenylphosphonium chloride (197.66 g, 576.61 mmol) in THE (1000 mL) was added Potassium tert-butoxide (69.04 g, 615.05 mmol) at 0° C. The reaction mixture was stirred for 12 hr at room temperature. The residue was added between ethyl acetate (2000 mL) and H₂O (1000 mL). The organic layer was separated, dried over Na₂SO₄, and were concentrated in vacuo. Then the residue was added a mixture of EtOAc and hexane (1:20, 3000 mL). The mixture was filtered, and the filtrate were concentrated in vacuo. The residue was purified by column chromatography to afford 2-[2-Ethyl-3-(2-methoxy-vinyl)-phenyl]-4,4,5,5-tetramethyl-[1,3,2]dioxaborolane (D61) (82 g). MS (ES): C₁₇H₂₅BO₃ requires 288; found 289 (M÷H⁺).

### Description for D62

2-chloro-5-{2-ethyl-3-[(E)-2-(methyloxy)ethenyl]phenyl}pyrimidine (D62)

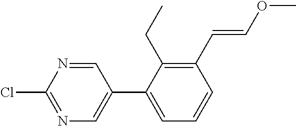 [SMILES: CCc1c(/C=C/OC)cccc1-c1cnc(Cl)nc1]

To a solution of 5-bromo-2-chloropyrimidine (2.014 g, 10.41 mmol), 2-{2-ethyl-3-[(E)-2-(methyloxy)ethenyl]phenyl}-4,4,5,5-tetramethyl-1,3,2-dioxaborolane (D61) (1.5 g, 5.20 mmol) and tripotassium phosphate (2.76 g, 13.01 mmol) in N,N-Dimethylformamide (DMF) (15 mL) and Water (3.75 mL) stirred under nitrogen at room temp° C. was added Pd(Ph₃P)₄ (0.601 g, 0.520 mmol) in one charge. The reaction vessel was sealed and heated in Biotage Initiator using initial high to 120° C. for 15 min. After cooling the reaction, LCMS showed the reaction was completed. The reaction mixture was filtered and the filtrate was partitioned between ethyl acetate (600 mL) and saturated brine (150 mL). The organic phase was dried over sodium sulphate and evaporated in vacuo. The crude product was purified by column chromatography to afford 2-chloro-5-{2-ethyl-3-[(E)-2-(methyloxy)ethenyl]phenyl}pyrimidine (D62) (1.13 g). MS (ES): C₁₅H₁₅ClN₂O requires 274.1; found 275.1 (M+H⁺),

### Description for D63

2-{3-chloro-4-[(1-methylethyl)oxy]phenyl}-5-{2-ethyl-3-[(E)-2-(methyloxy)ethenyl]phenyl}pyrimidine (D63)

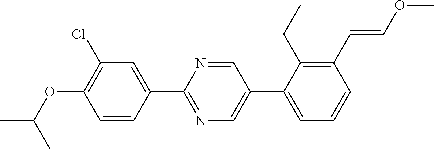 [SMILES: CCc1c(/C=C/OC)cccc1-c1cnc(-c2ccc(OC(C)C)c(Cl)c2)nc1]

To a solution of {3-chloro-4-[(1-methylethyl)oxy]phenyl}boronic acid (539 mg, 2.51 mmol), 2-chloro-5-{2-ethyl-3-[(E)-2-(methyloxy)ethenyl]phenyl}pyrimidine (D62) (460 mg, 1.674 mmol) and cesium carbonate (1364 mg, 4.19 mmol) in 1,2-Dimethoxyethane (DME) (10 mL) and Water (2.500 mL) stirred under nitrogen at room temp° C. was added Pd(Ph3P)₄ (97 mg, 0.084 mmol) in one charge. The reaction vessel was sealed and heated in Biotage Initiator using initial high to 120° C. for 15 min. After cooling the reaction, the reaction mixture was filtered and the filtrate was partitioned between ethyl acetate and saturated brine. The organic phase was dried over sodium sulphate and evaporated in vacuo. The crude product was purified by column chromatography to afford 2-{3-chloro-4-[(1-methylethyl)oxy]phenyl}-5-{2-ethyl-3-[(E)-2-(methyloxy)ethenyl]phenyl}pyrimidine (D63) (0.8 g). MS (ES): C₂₄H₂₅ClN₂O₂ requires 408.2; found 409.2 (M+H⁺).

### Description for D64

[3-(2-{3-chloro-4-[(1-methylethyl)oxy]phenyl}-5-pyrimidinyl)-2-ethylphenyl]acetaldehyde (D64)

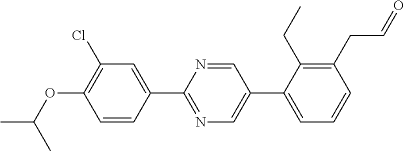 [SMILES: CCc1c(CC=O)cccc1-c1cnc(-c2ccc(OC(C)C)c(Cl)c2)nc1]

To a solution of 2-{3-chloro-4-[(1-methylethyl)oxy]phenyl}-5-{2-ethyl-3-[(E)-2-(methyloxy)ethenyl]phenyl}pyrimidine (D63) (0.8 g, 1.956 mmol) in Tetrahydrofuran (THF) (30 mL) stirred under nitrogen at room temp° C. was added HCl (2 mL, 4.00 mmol) in one charge. The reaction mixture was heated to 70° C. for 6 h. After cooling the reaction, the reaction mixture was concentrated in vacuo to afford [3-(2-{3-chloro-4-[(1-methylethyl)oxy]phenyl}-5-pyrimidinyl)-2-ethylphenyl]acetaldehyde (D64) (1 g), which was used for next step without further purification. MS (ES): C₂₃H₂₃ClN₂O₂ requires 394.1; found 395.1 (M+H⁺).

### Description for D65

5-(5-(2-ethyl-3-[(E)-2-(methyloxy)ethenyl]phenyl}-2-pyrimidinyl)-2-(2-methyl propyl)benzonitrile (D65)

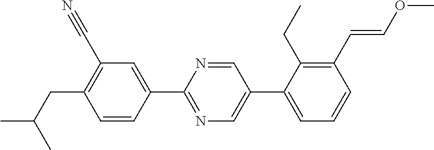 [SMILES: CCc1c(/C=C/OC)cccc1-c1cnc(-c2ccc(CC(C)C)c(C#N)c2)nc1]

To a solution of [(methyloxy)methyl](triphenyl)phosphonium chloride (835 mg, 2.436 mmol) in Tetrahydrofuran (THF) (20 mL) at −78° C., n-BuLi (1.522 mL, 2.436 mmol) was added slowly. The resulting solution was stirred for 20 mins at −78° C., then for 20 mins at room temperature. The reaction mixture was cooled down to −78° C. A solution of 5-[5-(2-ethyl-3-formylphenyl)-2-pyrimidinyl]-2-(2-methylpropyl)benzonitrile (D56) (600 mg, 1.624 mmol) in THF (5 mL) was added to the above reaction mixture slowly. The reaction was quenched with saturated NH4Cl. The residue was extracted with EA. The organic layer was combined, dried over Na2SO4, and evaporate in vacuo. The crude product was purified by column chromatography to afford 5-(5-{2-ethyl-3-[(E)-2-(methyloxy)ethenyl]phenyl}-2-pyrimidinyl)-2-(2-methyl propyl)benzonitrile (D65) (292 mg). MS (ES): C₂₆H₂₇N₃O requires 397.2; found 398.2 (M÷H⁺).

### Description for D66

5-{5-[2-ethyl-3-(2-oxoethyl)phenyl]-2-pyrimidinyl}-2-(2-methylpropyl)benzonitrile (D66)

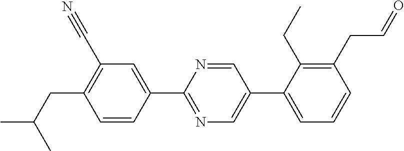 [SMILES: CCc1c(CC=O)cccc1-c1cnc(-c2ccc(CC(C)C)c(C#N)c2)nc1]

5-(5-(2-ethyl-3-[(E)-2-(methyloxy)ethenyl]phenyl}-2-pyrimidinyl)-2-(2-methylpropyl)benzonitrile (D65) (420 mg, 1.057 mmol) was dissolved in 10 ml acetonitrile, and then sodium iodide (317 mg, 2.113 mmol) was added. The resulting solution was cooled down to 0° C., and TMSCl (0.270 mL, 2.113 mmol) was added slowly. The reaction mixture was stirred for 20 mins. The reaction was quenched with water. CH₃CN was evaporated in vacuo. The resulting solution was washed with EA. The organic layer was combined, dried over Na₂SO₄, evaporated in vacuo to afford 5-{5-[2-ethyl-3-(2-oxoethyl)phenyl]-2-pyrimidinyl}-2-(2-methylpropyl)benzonitrile (D66) (180 mg), which was used for next step without further purification. MS (ES): C₂₅H₂₅N₃O requires 383.2; found 384.1 (M+H⁺).

### Description for D67

ethyl 4-{[2-ethyl-3-(4,4,5,5-tetramethyl-1,3,2-dioxaborolan-2-yl)phenyl]oxy}butanoate (D67)

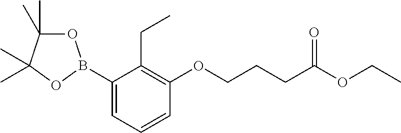 [SMILES: CCOC(=O)CCCOc1cccc(B2OC(C)(C)C(C)(C)O2)c1CC]

To a solution of 2-ethyl-3-(4,4,5,5-tetramethyl-1,3,2-dioxaborolan-2-yl)phenol (1 g, 4.03 mmol) and potassium carbonate (1.671 g, 12.09 mmol) in Acetonitrile (20 mL) stirred under nitrogen at room temperature was added ethyl 4-bromobutanoate (2.358 g, 12.09 mmol) in one charge. The reaction vessel was sealed and heated to 80° C. for 3 h. After cooling the reaction, the reaction mixture was partitioned between ethyl acetate and saturated brine. The organic phase was dried over sodium sulphate and evaporated in vacuo to give the crude product ethyl 4-{[2-ethyl-3-(4,4,5,5-tetramethyl-1,3,2-dioxaborolan-2-yl)phenyl]oxy}butanoate (D67) (1.46 g), which was used for next step without further purification. MS (ES): C₂₀H₃₁BO₅ requires 462.2; found 363.1 (M+H⁺).

### Description for D68

Ethyl 4-{[3-(2-{3-chloro-4-[(1-methylethyl)oxy]phenyl}-5-pyrimidinyl)-2-ethylphenyl]oxy}butanoate (D68)

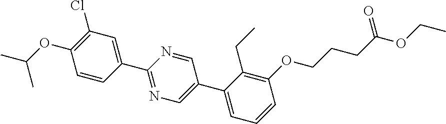 [SMILES: CCOC(=O)CCCOc1cccc(-c2cnc(-c3ccc(OC(C)C)c(Cl)c3)nc2)c1CC]

To a solution of 5-bromo-2-(3-chloro-4-[(1-methylethyl)oxy]phenyl}pyrimidine (D47) (180 mg, 0.549 mmol), ethyl 44{2-ethyl-3-(4,4,5,5-tetramethyl-1,3,2-dioxaborolan-2-yl)phenyl]oxy}butanoate (D67) (498 mg, 1.374 mmol) and tripotassium phosphate (292 mg, 1.374 mmol) in 1,2-Dimethoxyethane (DME) (4 mL) and Water (1.000 mL) stirred under nitrogen at room temperature was added Pd(Ph3P)₄ (31.7 mg, 0.027 mmol) in one charge. The reaction vessel was sealed and heated in Biotage Initiator using initial high to 120° C. for 15 min. After cooling the reaction, the reaction mixture was filtered and the filtrate was partitioned between ethyl acetate and saturated brine. The organic phase was dried over sodium sulphate and evaporated in vacuo to give the crude product. The crude product was purified by column chromatography to afford ethyl 4-([3-(2-{3-chloro-4-[(1-methylethyl)oxy]phenyl}-5-pyrimidinyl)-2-ethylphenyl]oxy}butanoate (D68) (200 mg). MS (ES): C₂₇H₃₁ClN₂O₄ requires 482.2; found 483.2 (M-4-H⁺).

### Description for D69

ethyl 4-{[3-(2-{3-cyano-4-(1-methylethyl)oxy]phenyl}-5-pyrimidinyl)-2-ethylphenyl]oxy}butanoate (D69)

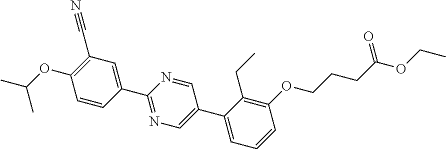 [SMILES: CCOC(=O)CCCOc1cccc(-c2cnc(-c3ccc(OC(C)C)c(C#N)c3)nc2)c1CC]

A mixture of 5-(5-bromo-pyrimidin-2-yl)-2-isopropoxy-benzonitrile (D57) (176 mg, 0.552 mmol), ethyl 4-{[2-ethyl-3-(4,4,5,5-tetramethyl-1,3,2-dioxaborolan-2-yl)phenyl]oxy}butanoate (D67) (200 mg, 0.552 mmol), Pd(Ph3P)₄ (63.8 mg, 0.055 mmol) and tripotassium phosphate (293 mg, 1.380 mmol) in 1,2-Dimethoxyethane (DME) (5.00 mL) and Water (1 mL) was heated at 120° C. in Biotage Initiator for 15 min. The mixture was extracted with ethyl acetate (50 mL). The organic layers were combined, dried over sodium sulfate, and evaporated in vacuo and purified by column chromatography to afford ethyl 4-{[3-(2-{3-cyano-4-[(1-methylethyl)oxy]phenyl}-5-pyrimidinyl)-2-ethylphenyl]oxy}butanoate (D69). MS (ES): C₂₈H₃₁N₃O₄ requires 473.2; found 474.1 (M+H⁺).

### Description for D70

ethyl 4-{[3-(2-{3-cyano-4-(1-methylethyl)oxy]phenyl}-5-pyrimidinyl)-2-ethyl phenyl]oxy}butanoate (D70)

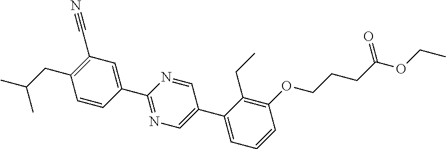 [SMILES: CCOC(=O)CCCOc1cccc(-c2cnc(-c3ccc(CC(C)C)c(C#N)c3)nc2)c1CC]

A mixture of 5-(5-bromo-pyrimidin-2-yl)-2-isobutyl-benzonitrile (D60) (175 mg, 0.552 mmol), ethyl 4-{[2-ethyl-3-(4,4,5,5-tetramethyl-1,3,2-dioxaborolan-2-yl)phenyl]oxy}butanoate (D67) (200 mg, 0.552 mmol), Pd(Ph3P)₄ (63.8 mg, 0.055 mmol) and tripotassium phosphate (293 mg, 1.380 mmol) in 1,2-Dimethoxyethane (DME) (10.00 mL) and Water (2 mL) was heated at 120° C. in Biotage Initiator for 15 min. The mixture was extracted with ethyl acetate (50 mL). The organic layers were combined, dried over sodium sulfate, and evaporated in vacuo and purified by column chromatography to afford ethyl 4-([3-(2-{3-cyano-4-[(1-methylethyl)oxy]phenyl}-5-pyrimidinyl)-2-ethyl phenyl]oxy}butanoate (D70) (220 mg). MS (ES): C₂₈H₃₁N₃O₃ requires 471.2; found 472.1 (M+H⁺).

### EXAMPLE 1

4-[3-(2-{3-cyano-4-[(1-methylethyl)oxy]phenyl}-5-pyrimidinyl)-2-ethylphenyl]butanoic acid (E1)

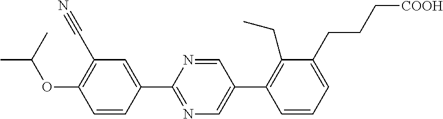 [SMILES: CCc1c(CCCC(=O)O)cccc1-c1cnc(-c2ccc(OC(C)C)c(C#N)c2)nc1]

To a solution of ethyl 4-[3-(2-{3-cyano-4-[(1-methylethyl)oxy]phenyl}-5-pyrimidinyl)-2-ethylphenyl]butanoate (D19) (200 mg) in isopropanol (20 mL) and water (5 mL) at room temperature was added NaOH (17.48 mg). The reaction mixture was stirred at room temperature for 3 h. The solvent was removed in vacuo. The residue was dissolved in water and the pH value was adjusted to about 6, then the mixture was partitioned between brine (10 mL) and THF (40 mL). The organic phase was concentrated and the residue was purified by Mass Directed Auto Prep to give 4-[3-(2-{3-cyano-4-[(1-methylethyl)oxy]phenyl}-5-pyrimidinyl)-2-ethylphenyl]butanoic acid (E1) (178 mg) as white solid. δH (DMSO-d₆, 400 MHz): 0.86 (3H, t), 1.32 (6H, d), 1.75 (2H, m), 2.28 (2H, t), 2.52 (2H, m), 2.63 (2H, t), 4.86 (1H, m), 7.05 (1H, d), 7.22 (2H, m), 7.42 (1H, d), 8.59 (2H, m), 8.82 (2H, s), 12.06 (1H, br s). MS (ES): C₂₆H₂₇N₃O₃ requires 429; found 430.2 (M+H⁺).

### EXAMPLE 2

4-(2-ethyl-3-{2-[4-[(1-methylethyl)oxy]-3-(trifluoromethyl)phenyl]-5-pyrimidinyl}phenyl)butanoic acid (E2)

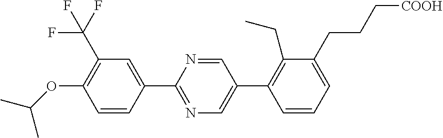 [SMILES: CCc1c(CCCC(=O)O)cccc1-c1cnc(-c2ccc(OC(C)C)c(C(F)(F)F)c2)nc1]

To a solution of ethyl 4-(2-ethyl-3-{2-[4-[(1-methylethyl)oxy]-3-(trifluoromethyl)phenyl]-5-pyrimidinyl}phenyl)butanoate (D20) (105 mg) in isopropanol (3 mL) and water (0.750 mL) stirred at room temperature was added 2M NaOH (0.105 mL) in one charge. The reaction mixture was stirred at room temperature for 2 h. The solvent was removed in vacuo. The residue was dissolved in water and the pH value was adjusted to about 6. Then the mixture was partitioned between brine (10 mL) and THF (40 mL). The organic phase was concentrated and the residue was purified by Mass Directed Auto Prep to give 4-(2-ethyl-3-{2-[4-[(1-methylethyl)oxy]-3-(trifluoromethyl)phenyl]-5-pyrimidinyl}phenyl)butanoic acid (E2) (50 mg). δH (DMSO-d₆, 400 MHz): 0.86 (3H, t), 1.28 (6H, d), 1.76 (2H, m), 2.28 (2H, t), 2.52 (2H, m), 2.63 (2H, t), 4.86 (1H, m), 7.05 (1H, d), 7.22 (2H, m), 7.44 (1H, d), 8.59 (2H, m), 8.82 (2H, s), 12.07 (1H, br s). δF (DMSO-d₆, 376 MHz): −61.2. MS (ES): C₂₆H₂₇F₃N₂O₃ requires 472; found 473.2 (M+H⁺).

### EXAMPLE 3

4-(2-ethyl-3-{2-[6-[(1-methylethyl)oxy]-5-(trifluoromethyl)-3-pyridinyl]-5-pyrimidinyl}phenyl)butanoic acid (E3)

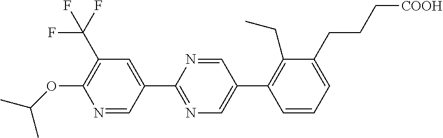 [SMILES: CCc1c(CCCC(=O)O)cccc1-c1cnc(-c2cnc(OC(C)C)c(C(F)(F)F)c2)nc1]

To a solution of ethyl 4-(2-ethyl-3-{2-[6-[(1-methylethyl)oxy]-5-(trifluoromethyl)-3-pyridinyl]-5-pyrimidinyl}phenyl)butanoate (D21) (65 mg) in isopropanol (3 mL) and water (0.750 mL) stirred at room temperature was added 2M NaOH (0.233 mL) in one charge. The reaction mixture was stirred at room temperature for 2 h. The solvent was removed in vacuo. The residue was dissolved in water and the pH value was adjusted to about 6. Then the mixture was partitioned between brine (10 mL) and THF (40 mL). The organic phase was concentrated and the residue was purified by Mass Directed Auto Prep to give 4-(2-ethyl-3-{2-[6-[(1-methylethyl)oxy]-5-(trifluoromethyl)-3-pyridinyl]-5-pyrimidinyl}phenyl)butanoic acid (E3) (5 mg). δH (DMSO-d₆, 400 MHz): 0.86 (3H, t), 1.32 (6H, d), 1.76 (2H, m), 2.28 (2H, t), 2.52 (2H, m), 2.63 (2H, t), 5.48 (1H, m), 7.05 (1H, d), 7.22 (2H, m), 8.83 (1H, d), 8.87 (2H, s), 9.33 (1H, d), 12.05 (1H, br s). δF (DMSO-d₆, 376 MHz): −62.7. MS (ES): C₂₅H₂₆F₃N₃O₃ requires 473; found 474.2 (M+H⁺).

### EXAMPLE 4

4-[3-(2-{3-chloro-4-[(1-methylethyl)oxy]phenyl}-5-pyrimidinyl)-2-ethylphenyl]butanoic acid (E4)

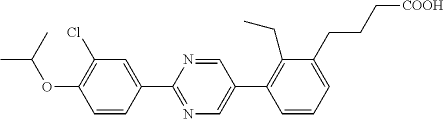 [SMILES: CCc1c(CCCC(=O)O)cccc1-c1cnc(-c2ccc(OC(C)C)c(Cl)c2)nc1]

To a solution of ethyl 4-[3-(2-{3-chloro-4-[(1-methylethyl)oxy]phenyl}-5-pyrimidinyl)-2-ethylphenyl]butanoate (D22) (240 mg) in isopropanol (3 mL) and water (0.750 mL) stirred under nitrogen at room temperature was added 20% NaOH solution (216 mg) in one charge. The reaction mixture was stirred at rt for 2 h. The solvent was removed in vacuo. The residue was dissolved in water and the pH value was adjusted to about 6. Then the mixture was partitioned between brine (10 mL) and THF (40 mL). The organic phase was concentrated and the residue was purified by Mass Directed AutoPrep to give 4-[3-(2-{3-chloro-4-[(1-methylethyl)oxy]phenyl}-5-pyrimidinyl)-2-ethylphenyl]butanoic acid (E4) (31 mg). δH (DMSO-d₆, 400 MHz): 0.86 (3H, t), 1.28 (6H, d), 1.75 (2H, m), 2.28 (2H, t), 2.52 (2H, m), 2.62 (2H, t), 4.76 (1H, m), 7.04 (1H, d), 7.21 (2H, m), 7.29 (1H, d), 8.30 (1H, d), 8.36 (1H, d), 8.79 (2H, s), 12.12 (1H, br s). MS (ES): C₂₅H₂₇ClN₂O₃ requires 438; found 439.2 (M+W).

### EXAMPLE 5

4-[5-fluoro-3-{2-[4-[(1-methylethyl)oxy]-3-(trifluoromethyl)phenyl]-5-pyrimidinyl}-2-(methyloxy)phenyl]butanoic acid (E5)

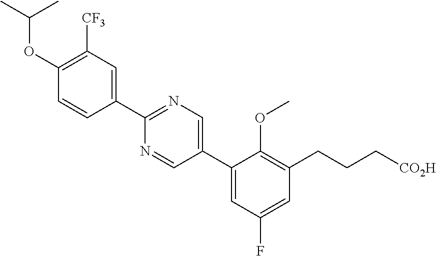 [SMILES: COc1c(CCCC(=O)O)cc(F)cc1-c1cnc(-c2ccc(OC(C)C)c(C(F)(F)F)c2)nc1]

To a solution of ethyl 4-[5-fluoro-3-{2-[4-[(1-methylethyl)oxy]-3-(trifluoromethyl)phenyl]-5-pyrimidinyl}-2-(methyloxy)phenyl]butanoate (D29) (40 mg) in isopropanol (8 mL) and water (2 mL) was added NaOH (3.07 mg). The reaction mixture was stirred at room temperature for 5 h. The mixture was concentrated, the residue was dissolved in water and the pH value was adjusted to about 7, then the mixture was partitioned between brine (10 mL) and THF (40 mL). The organic phase was concentrated and the residue was purified by Mass Directed AutoPrep to give 4-[5-fluoro-3-{2-[4-[(1-methylethyl)oxy]-3-(trifluoromethyl)phenyl]-5-pyrimidinyl}-2-(methyloxy)phenyl]butanoic acid (E5) (12 mg) as a white solid. δH (DMSO-d₄, 400 MHz): 1.34 (6H, d), 1.85 (2H, m), 2.29 (2H, t), 2.69 (2H, t), 3.37 (3H, s), 4.91 (1H, m), 7.21 (1H, dd), 7.34 (1H, dd), 7.48 (1H, d), 8.66 (2H, m), 9.09 (2H, s), 12.11 (1H, br s). δF (DMSO-d₆, 376 MHz): −117.8, −61.2. MS (ES): C₂₅H₂₄F₄N₂O₄ requires 492; found 493.2 (M+H⁺).

### EXAMPLE 6

4-[3-(2-{3-cyano-4-[(1-methylethyl)oxy]phenyl}-5-pyrimidinyl)-5-fluoro-2-(methyloxy)phenyl]butanoic acid (E6)

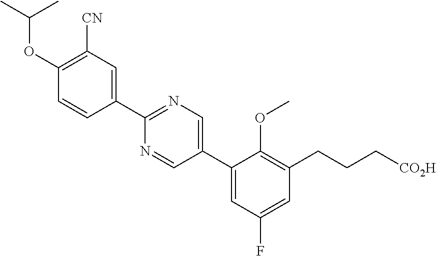 [SMILES: COc1c(CCCC(=O)O)cc(F)cc1-c1cnc(-c2ccc(OC(C)C)c(C#N)c2)nc1]

To a solution of ethyl 4-[3-(2-{3-cyano-4-[(1-methylethyl)oxy]phenyl}-5-pyrimidinyl)-5-fluoro-2-(methyloxy)phenyl]butanoate (D30) (40 mg) in isopropanol (8 mL) and water (2 mL) was added NaOH (6.7 mg). The reaction mixture was stirred at room temperature for 4 h. The mixture was concentrated, the residue was dissolved in water and the pH value was adjusted to about 7, then the mixture was partitioned between brine (10 mL) and THF (40 mL). The organic phase was concentrated and the residue was purified by Mass Directed AutoPrep to give 4-[3-(2-{3-cyano-4-[(1-methylethyl)oxy]phenyl}-5-pyrimidinyl)-5-fluoro-2-(methyloxy)phenyl]butanoic acid (E6) (19 mg) as white solid. δH (DMSO-d₆, 400 MHz): 1.36 (6H, d), 1.85 (2H, m), 2.31 (2H, t), 2.69 (2H, t), 3.36 (3H, s), 4.92 (1H, m), 7.21 (1H, dd), 7.34 (1H, dd), 7.46 (1H, d), 8.65 (2H, m), 9.09 (2H, s), 12.10 (1H, br s). δF (DMSO-d₆, 376 MHz): −117.8, −73.5. MS (ES): C₂₅H₂₄FN₃O₄ requires 449; found 450.1 (M+H⁺).

### EXAMPLE 7

4-[3-(2-{3-cyano-4-[(1-methylethyl)oxy]phenyl}-5-pyrimidinyl)-2-(methyloxy)phenyl]butanoic acid (E7)

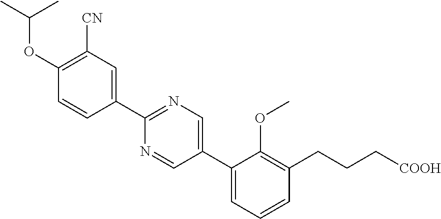 [SMILES: COc1c(CCCC(=O)O)cccc1-c1cnc(-c2ccc(OC(C)C)c(C#N)c2)nc1]

To the mixture of ethyl 4-[3-(2-{3-cyano-4-[(1-methylethyl)oxy]phenyl}-5-pyrimidinyl)-2-(methyloxy)phenyl]butanoate (D30) (60 mg) in isopropanol (2.5 mL) and water (2.500 mL) was added lithium hydroxide (10.96 mg). The mixture was stirred at 79° C. for 2 h. After cooling the reaction, the reaction mixture was neutralized with AcOH and evaporated, the residue was purified by Mass Directed AutoPrep to give 4-[3-(2-{3-cyano-4-[(1-methylethyl)oxy]phenyl}-5-pyrimidinyl)-2-(methyloxy)phenyl]butanoic acid (E7) (38 mg). δH (DMSO-d₆, 400 MHz): 1.32 (6H, d), 1.79 (2H, m), 2.25 (2H, t), 2.63 (2H, t), 3.28 (3H, s), 4.86 (1H, m), 7.18 (1H, t), 7.28 (111, d), 7.35 (111, d), 7.40 (1H, d), 8.80 (2H, m), 9.01 (2H, s), 12.03 (1H, br s). δF (DMSO-d₆, 376 MHz): −73.5. MS (ES): C₂₅H₂₅N₃O₄ requires 431; found 432.1 (M+H⁴).

### EXAMPLE 8

N-[(2-ethyl-3-{2-[4-[(1-methylethyl)oxy]-3-(trifluoromethyl)phenyl]-5-pyrimidinyl}phenyl)methyl]-N-methylglycine (E8)

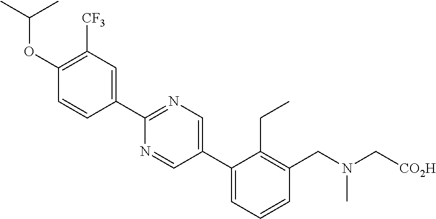 [SMILES: CCc1c(CN(C)CC(=O)O)cccc1-c1cnc(-c2ccc(OC(C)C)c(C(F)(F)F)c2)nc1]

To a mixture of 2-ethyl-3-{2-[4-[(1-methylethyl)oxy]-3-(trifluoromethyl)phenyl]-5-pyrimidinyl}benzaldehyde (D39) (70 mg) and N-methylglycine (150 mg) in ethanol (30.00 mL) at room temperature was added acetic acid (9.67 μL). The reaction mixture was heated to 80° C. for 5 h. The solvent was removed in vacuo and dichloromethane (DCM) (30 mL) was added into the residue followed by sodium triacetoxyborohydride (107 mg). The mixture was stirred at room temperature overnight. Sat. NaHCO₃ (5 mL) was added into the reaction mixture and the resulting solution was stirred for 5 min. The mixture was partitioned between brine (20 mL) and EtOAc (100 mL). The organic phase was concentrated and the residue was purified by Mass Directed AutoPrep to give N-[(2-ethyl-3-{2-[4-[(1-methylethyl)oxy]-3-(trifluoromethyl)phenyl]-5-pyrimidinyl}phenyl)methyl]-N-methylglycine (E8) (7 mg) as a white solid. δH (DMSO-d₆, 400 MHz): 0.86 (3H, t), 1.27 (6H, d), 2.27 (3H, s), 2.44 (2H, s), 2.60 (2H, m), 3.72 (2H, s), 4.86 (1H, m), 7.15 (1H, d), 7.25 (1H, t), 7.41 (2H, m), 8.60 (2H, m), 8.82 (2H, s). δF (DMSO-d₆, 376 MHz): −61.2. MS (ES): C₂₆H₂₈F₃N₃O₃ requires 487; found 488.3 (M+H⁺).

### EXAMPLE 9

N-ethyl-N-[(2-ethyl-3-{2-(4-[(1-methylethyl)oxy]-3-(trifluoromethyl)phenyl]-5-pyrimidinyl}phenyl)methyl]glycine (E9)

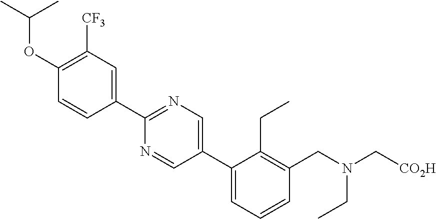 [SMILES: CCc1c(CN(CC)CC(=O)O)cccc1-c1cnc(-c2ccc(OC(C)C)c(C(F)(F)F)c2)nc1]

To a mixture of 2-ethyl-3-{2-[4-[(1-methylethyl)oxy]-3-(trifluoromethyl)phenyl]-5-pyrimidinyl}benzaldehyde (D39) (70 mg) and N-ethylglycine (174 mg) in ethanol (20 mL) at room temperature was added acetic acid (9.67 μL). The reaction mixture was heated to 80° C. for 1 h. The solvent was removed in vacuo and dichloromethane (DCM) (30 mL) was added into the residue followed by sodium triacetoxyborohydride (107 mg). The mixture was stirred at room temperature overnight. Sat. NaHCO₃ (5 mL) was added into the reaction mixture and the resulting solution was stirred for 5 min. The mixture was partitioned between brine (20 mL) and EtOAc (100 mL). The organic phase was concentrated and the residue was purified by Mass Directed AutoPrep to give N-ethyl-N-[(2-ethyl-3-{2-[4-[(1-methylethyl)oxy]-3-(trifluoromethyl)phenyl]-5-pyrimidinyl}phenyl)methyl]glycine (E9) (23 mg) as a white solid. δH (DMSO-d₅, 400 MHz): 0.87 (3H, t), 1.15 (3H, m), 1.28 (6H, d), 2.64 (2H, m), 3.05 (2H, s), 3.84 (2H, m), 4.26 (2H, s), 4.86 (1H, m), 7.28 (1H, d), 7.38 (1H, t), 7.42 (1H, d), 7.56 (1H, d), 8.60 (2H, m), 8.83 (2H, s). δF (DMSO-d₆, 376 MHz): −73.8, −61.2. MS (ES): C₂₇H₃₀F₃N₃O₃ requires 501; found 502.1 (M+H⁺).

### EXAMPLE 10

N-[(2-ethyl-3-{2-[4-[(1-methylethyl)oxy]-3-(trifluoromethyl)phenyl]-5-pyrimidinyl}phenyl)methyl]-b-alanine (E10)

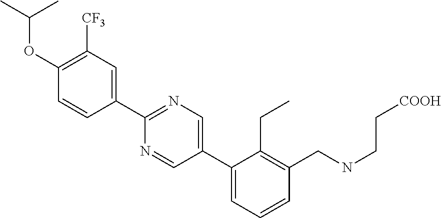 [SMILES: CCc1c(CNCCC(=O)O)cccc1-c1cnc(-c2ccc(OC(C)C)c(C(F)(F)F)c2)nc1]

To a mixture of ethyl N-[(2-ethyl-3-{2-[4-[(1-methylethyl)oxy]-3-(trifluoromethyl)phenyl]-5-pyrimidinyl}phenyl)methyl]-β-alaninate (D40) (50 mg) in isopropanol (20 mL) and water (10 mL) at room temperature was added NaOH (7.76 mg). After stirring for 5 h, the mixture was concentrated, the residue was dissolved in water and the pH value was adjusted to about 6, the resulting solution was concentrated and the residue was purified by Mass Directed AutoPrep to give N-[(2-ethyl-3-{2-[4-[(1-methylethyl)oxy]-3-(trifluoromethyl)phenyl]-5-pyrimidinyl}phenyl)methyl]-β-alanine (E10) (18 mg) as a white solid. δH (DMSO-d₆, 400 MHz): 0.91 (3H, t), 1.28 (6H, d), 2.58 (2H, m), 2.66 (2H, t), 3.25 (2H, s), 4.26 (2H, s), 4.86 (1H, m), 7.30 (1H, t), 7.41 (2H, m), 7.54 (1H, d), 8.58 (2H, m), 8.80 (2H, s), 12.69 (1H, br s). δF (DMSO-d₆, 376 MHz): −73.6, −61.2. MS (ES): C₂₈H₂₈F₃N₃O₃ requires 487; found 488.3 (M+H⁺).

### EXAMPLE 11

1-[(2-ethyl-3-{2-[4-[(1-methylethyl)oxy]-3-(trifluoromethyl)phenyl]-5-pyrimidinyl}phenyl)methyl]-3-azetidinecarboxylic acid (E11)

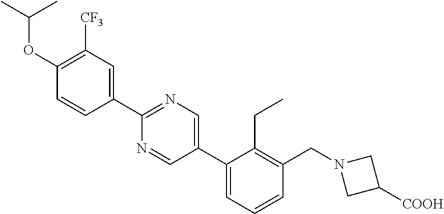 [SMILES: CCc1c(CN2CC(C(=O)O)C2)cccc1-c1cnc(-c2ccc(OC(C)C)c(C(F)(F)F)c2)nc1]

To a mixture of 2-ethyl-3-{2-[4-[(1-methylethyl)oxy]-3-(trifluoromethyl)phenyl]-5-pyrimidinyl}benzaldehyde (D39) (60 mg) and 3-azetidinecarboxylic acid (58.6 mg) in dichloromethane (DCM) (10 mL) at room temperature was added acetic acid (8.29 μL). After stirring for 1 h, sodium triacetoxyborohydride (77 mg) was added to the reaction mixture. The resulting suspension was stirred at room temperature overnight. Sat. NaHCO₃ (5 mL) was added into the reaction mixture and the resulting solution was stirred for 5 min. The mixture was partitioned between brine (20 mL) and EtOAc (100 mL). The organic phase was concentrated and purified by Mass Directed AutoPrep to give 1-[(2-ethyl-3-{2-[4-[(1-methylethyl)oxy]-3-(trifluoromethyl)phenyl}-5-pyrimidinyl}phenyl)methyl]-3-azetidinecarboxylic acid (E11) (14 mg). δH (DMSO-d₆, 400 MHz): 0.89 (3H, t), 1.27 (6H, d), 2.54 (2H, m), 3.20 (5H, m), 3.59 (2H, s), 4.84 (1H, m), 7.10 (1H, d), 7.22 (1H, t), 7.34 (1H, d), 7.42 (1H, d), 8.59 (2H, m), 8.81 (2H, s), 12.26 (1H, br s). δF (DMSO-d₆, 376 MHz): −61.2. MS (ES): C₂₇H₂₈F₃N₃O₃ requires 499; found 500.3 (M+H⁺).

### EXAMPLE 12

1-{[(2-ethyl-3-{2-[4-[(1-methylethyl)oxy]-3-(trifluoromethyl)phenyl]-5-pyrimidinyl}phenyl)methyl]amino}cyclopropanecarboxylic acid (E12)

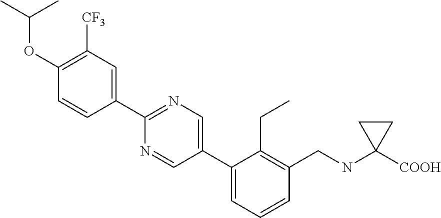 [SMILES: CCc1c(CNC2(C(=O)O)CC2)cccc1-c1cnc(-c2ccc(OC(C)C)c(C(F)(F)F)c2)nc1]

To a mixture of 2-ethyl-3-{2-[4-[(1-methylethyl)oxy]-3-(trifluoromethyl)phenyl]-5-pyrimidinyl}benzaldehyde (D39) (80 mg) and 1-aminocyclopropanecarboxylic acid (78 mg) in ethanol (30.00 mL) at room temperature was added acetic acid (55 μA). After stirring for 20 min, sodium triacetoxyborohydride (102 mg) was added to the reaction mixture. The resulting suspension was stirred at room temperature overnight. Sat. NaHCO₃ (5 mL) was added into the reaction mixture and the resulting solution was stirred for 5 min. The mixture was partitioned between brine (20 mL) and EtOAc (100 mL). The organic phase was concentrated and purified by Mass Directed AutoPrep to give 1-{[(2-ethyl-3-{2-[4-[(1-methylethyl)oxy]-3-(trifluoromethyl)phenyl]-5-pyrimidinyl}phenyl)methyl]amino]cyclopropanecarboxylic acid (E12) (14 mg). δH (DMSO-d₅, 400 MHz): 0.92 (3H, t), 1.28 (6H, d), 1.42 (4H, m), 2.66 (2H, m), 4.33 (2H, s), 4.86 (1H, m), 7.29 (1H, d), 7.37 (1H, t), 7.45 (1H, d), 7.53 (1H, d), 8.59 (2H, m), 8.81 (2H, s). δF (DMSO-d₅, 376 MHz): −73.7, −61.2. MS (ES): C₂₇H₂₈F₃N₃O₃ requires 499; found 500.1 (M+H⁺).

### EXAMPLE 13

1-[(2-ethyl-3-{2-[4-[(1-methylethyl)oxy]-3-(trifluoromethyl)phenyl]-5-pyrimidinyl}phenyl)methyl]-4-piperidinecarboxylic acid (E13)

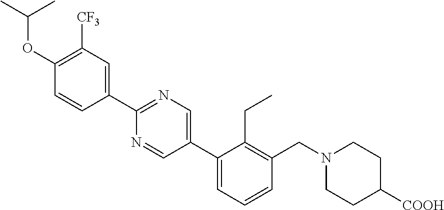 [SMILES: CCc1c(CN2CCC(C(=O)O)CC2)cccc1-c1cnc(-c2ccc(OC(C)C)c(C(F)(F)F)c2)nc1]

To a mixture of ethyl 1-[(2-ethyl-3-{2-[4-(1-methylethyl)oxy]-3-(trifluoromethyl)phenyl]-5-pyrimidinyl}phenyl)methyl]-4-piperidinecarboxylate (D31) (70 mg) in isopropanol (20 mL) and water (8 mL) at room temperature was added NaOH (10.08 mg). After stirring for 3 h, the mixture was concentrated, the residue was dissolved in water and the pH value was adjusted to about 6, the resulting solution was concentrated and the residue was purified by Mass Directed AutoPrep to give 1-[(2-ethyl-3-{2-[4-[(1-methylethyl)oxy]-3-(trifluoromethyl)phenyl]-5-pyrimidinyl}phenyl)methyl]-4-piperidinecarboxylic acid (E13) (30 mg) as a white solid. 5H (DMSO-d₆, 400 MHz): 0.83 (3H, t), 1.28 (6H, d), 1.71 (2H, m), 2.01 (2H, m), 2.63 (2H, m), 3.10 (2H, m), 3.28 (2H, m), 4.36 (2H, m), 4.86 (1H, m), 7.33 (1H, d), 7.42 (2H, m), 7.58 (1H, d), 8.61 (2H, m), 8.85 (2H, s), 9.13 (1H, s), 12.53 (1H, br s). δF (DMSO-d₆, 376 MHz): −73.6, −61.2. MS (ES): C₂₉H₃₂F₃N₃O₃ requires 527; found 528.3 (M+W).

### EXAMPLE 14

4-(3-{2-[3-cyano-4-(2-methylpropyl)phenyl]-5-pyrimidinyl}-2-ethylphenyl) butanoic acid (E14)

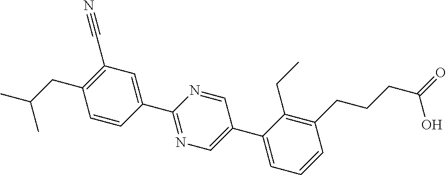 [SMILES: CCc1c(CCCC(=O)O)cccc1-c1cnc(-c2ccc(CC(C)C)c(C#N)c2)nc1]

To a solution of ethyl 4-(3-{2-{3-cyano-4-(2-methylpropyl)phenyl}-5-pyrimidinyl}-2-ethylphenyl)butanoate (D46) (75 mg, 0.165 mmol) in Isopropanol (4 mL) and Water (1.000 mL) stirred under nitrogen at room temperature was added NaOH (0.5 mL, 1.000 mmol) in one charge. The reaction mixture was stirred at room temperature for 2 h. Isopropanol was removed in vacuo. The residue was dissolved in water and acidified with 1N HCl aq. solution to pH=5. The samples were dissolved in THF (6 mL) and purified by Mass Directed AutoPrep. The solvent was freeze dried to afford 4-(3-{2-[3-cyano-4-(2-methylpropyl)phenyl]-5-pyrimidinyl}-2-ethylphenyl)butanoic acid (E14) (16 mg). δH (DMSO-d₆, 400 MHz): 0.93 (9H, m) 1.82 (2H, m) 1.99 (1H, m) 2.34 (2H, t) 2.58 (2H, m) 2.72 (4H, m) 7.12 (1H, d) 7.28 (2H, m) 7.64 (1H, d) 8.66 (2H, m) 8.93 (2H, s) 12.07 (1H, br. s.). MS (ES): C₂₇H₂₉N₃O₂ requires 427.2; found 428.2 (M+H⁺).

### EXAMPLE 15

3-{4-[3-(2-{3-chloro-4-[(1-methylethyl)oxy]phenyl}-5-pyrimidinyl)-2-ethylphenyl]-1-piperidinyl}propanoic acid trifluoroacetate (E15)

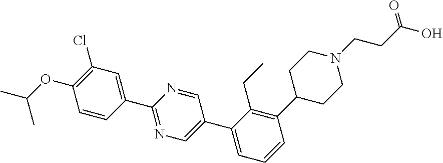 [SMILES: CCc1c(-c2cnc(-c3ccc(OC(C)C)c(Cl)c3)nc2)cccc1C1CCN(CCC(=O)O)CC1]

To a solution of ethyl 3-{4-[3-(2-}3-chloro-4-[(1-methylethyl)oxy]phenyl}-5-pyrimidinyl)-2-ethylphenyl]-1-piperidinyl}propanoate (D53) (120 mg, 0.224 mmol) in Isopropanol and Water stirred under nitrogen at room temperature was added NaOH (179 mg, 0.895 mmol) in one charge. The reaction mixture was stirred at room temperature for 2 h. Isopropanol was removed in vacuo. The residue was dissolved in water and acidified with 1N HCl aq. solution to pH=6. The samples were dissolved in THF (6 mL) and purified by Mass Directed AutoPrep. The solvent was freeze dried to afford 3-{4-[3-(2-{3-chloro-4-[(1-methylethyl)oxy]phenyl}-5-pyrimidinyl)-2-ethylphenyl]-1-piperidinyl}propanoic acid (E15) (70 mg) as a TFA salt. δH (DMSO-d₅, 400 MHz): 1.00 (3H, t), 1.36 (6H, d), 1.94 (4H, m), 2.60 (2H, m), 2.81 (2H, m), 3.20 (3H, m), 3.35 (2H, m), 3.58 (2H, m), 4.83 (1H, m), 7.17 (1H, d), 7.34 (3H, m), 8.39 (2H, m), 8.84 (2H, s), 9.32 (1H, br. s.), 12.81 (1H, br. s.). δf (DMSO-d₅, 376 MHz): −73.5. MS (ES): C₂₉H₃₄ClN₃O₃ requires 507.2; found 508.3 (M+H⁺).

### EXAMPLE 16

4-{4-[3-(2-{3-chloro-4-[(1-methylethyl)oxy]phenyl}-5-pyrimidinyl)-2-ethylphenyl]-1-piperidinyl}butanoic acid trifluoroacetate (E16)

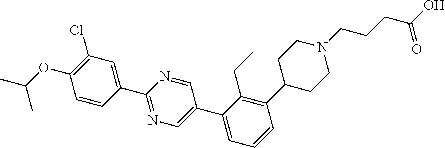 [SMILES: CCc1c(-c2cnc(-c3ccc(OC(C)C)c(Cl)c3)nc2)cccc1C1CCN(CCCC(=O)O)CC1]

To a solution of ethyl 4-{4-[3-(2-{3-chloro-4-[(1-methylethyl)oxy]phenyl}-5-pyrimidinyl)-2-ethylphenyl]-1-piperidinyl}butanoate (D54) (130 mg, 0.236 mmol) in Isopropanol (3 mL) and Water (1.000 mL) stirred under nitrogen at room temperature was added NaOH (189 mg, 0.945 mmol) in one charge. The reaction mixture was stirred at room temperature for 2 h. Isopropanol was removed in vacuo. The residue was dissolved in water and acidified with 1N HCl aq. solution to pH=6. The samples were dissolved in THF 6 mL and purified by Mass Directed AutoPrep. The solvent was freeze dried to afford 4-{4-[3-(2-{3-chloro-4-[(1-methylethyl)oxy]phenyl}-5-pyrimidinyl)-2-ethylphenyl]-1-piperidinyl}butanoic acid (E16) (81 mg) as a TFA salt. δH (DMSO-d₆, 400 MHz): 1.00 (3H, t) 1.35 (6H, d) 1.94 (6H, m) 2.37 (2H, m) 2.60 (2H, d) 3.11 (2H, m) 3.18 (3H, m) 3.60 (2H, m) 4.82 (1H, m) 7.17 (1H, d) 7.34 (3 H, m) 8.36 (1H, d) 8.43 (1H, s) 8.84 (2H, s) 9.30 (1H, br. s.) 12.37 (1H, br. s.). δF (DMSO-d₆, 376 MHz): −73.5, MS (ES): C₃₀H₃₆ClN₃O₃ requires 521.2; found 522.3 (M+H⁺)

### EXAMPLES 17-20

### EXAMPLE 17

N-{[3-(2-{3-chloro-4-[(1-methylethyl)oxy]phenyl}-5-pyrimidinyl)-2-ethylphenyl]methyl}-N-methylglycine trifluoroacetate (E17)

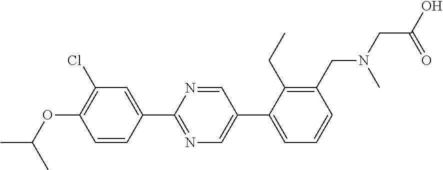 [SMILES: CCc1c(CN(C)CC(=O)O)cccc1-c1cnc(-c2ccc(OC(C)C)c(Cl)c2)nc1]

To a solution of 3-(2-{3-chloro-4-[(1-methylethyl)oxy]phenyl}-5-pyrimidinyl)-2-ethylbenzaldehyde (D55) (300 mg, 0.788 mmol) and sodium acetate (323 mg, 3.94 mmol) in Dichloromethane (DCM) (8 mL) stirred at room temperature was added ethyl N-methylglycinate (461 mg, 3.94 mmol) and acetic acid (0.15 mL, 2.62 mmol) in one charge. The reaction mixture was stirred at room temperature for 15 min. Then sodium triacetoxyborohydride (417 mg, 1.969 mmol) was added to the above mixture. The reaction mixture was stirred for another 2 h. The reaction mixture was partitioned between ethyl acetate and saturated brine. The organic phase was dried over sodium sulphate and evaporated in vacuo to afford ethyl N-{[3-(2-{3-chloro-4-[(1-methylethyl)oxy]phenyl}-5-pyrimidinyl)-2-ethylphenyl]methyl}-N-methylglycinate (436 mg). Then to a solution of ethyl N-{[3-(2-{3-chloro-4-[(1-methylethyl)oxy]phenyl}-5-pyrimidinyl)-2-ethylphenyl]methyl}-N-methylglycinate (436 mg, 0.787 mmol) in isopropanol (6 mL) and Water (2.000 mL) stirred under nitrogen at room temperature was added NaOH (630 mg, 3.15 mmol) in one charge. The reaction mixture was stirred at room temperature for 2 h. Isopropanol was removed in vacuo. The residue was dissolved in water and acidified with 1N HCl aq, solution to pH=5. The samples were dissolved in THF (6 mL) and purified by Mass Directed AutoPrep. The solvent was freeze dried to afford N-{[3-(2-{3-chloro-4-[(1-methylethyl)oxy]phenyl}-5-pyrimidinyl)-2-ethylphenyl]methyl}-N-methylglycine (E17) (202 mg) as a TFA salt. The following examples were prepared using procedures described for Example 17.

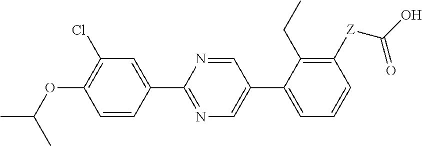

| Example | Z—COOH | characterization |
|---|---|---|
| 17 | 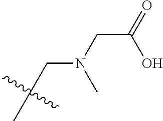 [SMILES: *CN(C)CC(=O)O] | δH (DMSO-d₆, 400 MHz): 0.90 (3 H, t), 1.36 (6 H, d), 2.75 (5 H, m), 4.09 (2 H, m), 4.38 (2 H, m), 4.83 (1 H, dt), 7.41 (3 H, m), 7.63 (1 H, d), 8.37 (1 H, dd), 8.44 (1 H, d), 8.87 (2 H, s). δF (DMSO-d₆, 376 MHz): −73.8. MS (ES): C₂₅H₂₈ClN₃O₃ requires 453.2; found 454.2 (M + H⁺). |
| 18 | 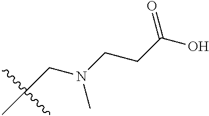 [SMILES: *CN(C)CCC(=O)O] | δH (DMSO-d₆, 400 MHz): 0.92 (3 H, t), 1.35 (6 H, d), 2.15 (3 H, s), 2.42 (3 H, m), 2.66 (4 H, m), 3.56 (2 H, s), 4.82 (1H, dt), 7.18 (1 H, d), 7.28 (1 H, t), 7.38 (2 H, m), 8.37 (1 H, dd), 8.43 (1 H, d), 8.86 (2 H, s). MS (ES): C₂₆H₃₀ClN₃O₃ requires 467.2; found 468.2 (M + H⁺). |
| 19 | 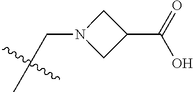 [SMILES: *CN1CC(C(=O)O)C1] | δH (DMSO-d₆, 400 MHz): 0.93 (3 H, t), 1.35 (6 H, d), 2.65 (2 H, m), 3.66 (2 H, m), 4.27 (4 H, m), 4.55 (2 H, m), 4.82 (1 H, dt), 7.39 (3 H, m), 7.51 (1 H, d), 8.37 (1 H, dd), 8.43 (1 H, d), 8.85 (2 H, s). δF (DMSO-d₆, 376 MHz): −73.5. MS (ES): C₂₆H₂₈ClN₃O₃ requires 465.2; found 466.2 (M + H⁺). |
| 20 | 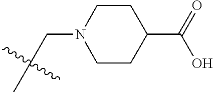 [SMILES: *CN1CCC(C(=O)O)CC1] | δH (DMSO-d₆, 400 MHz): 0.94 (3 H, t), 1.35 (6 H, d), 1.54 (2 H, m), 1.81 (1 H, m), 1.78 (1 H, m), 2.05 (2 H, m), 2.23 (1 H, m), 2.65 (2 H, m), 2.77 (2 H, m), 3.51 (2 H, s), 4.82 (1 H, m), 7.18 (1 H, d), 7.28 (1 H, t), 7.38 (2 H, m), 8.36 (1 H, dd), 8.43 (1 H, d), 8.86 (2 H, s), 12.14 (1 H, br. s.). MS (ES): C₂₆H₃₂ClN₃O₃ requires 493.2; found 494.2 (M + H⁺). |

### EXAMPLE 21-25

### EXAMPLE 21

N-[(3-{2-[3-cyano-4-(2-methylpropyl)phenyl]-5-pyrimidinyl}-2-ethylphenyl)methyl]-N-methylglycine trifluoroacetate (E21)

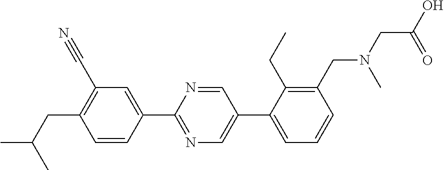 [SMILES: CCc1c(CN(C)CC(=O)O)cccc1-c1cnc(-c2ccc(CC(C)C)c(C#N)c2)nc1]

The mixture of 5-[5-(2-ethyl-3-formylphenyl)-2-pyrimidinyl]-2-(2-methylpropyl)benzonitrile (D56) (200 mg, 0.541 mmol), methyl N-methylglycinate, and acetic acid (0.093 mL, 1.624 mmol) in Dichloromethane (DCM) (4 mL) at it was stirred for 15 min at room temperature. sodium triacetoxyborohydride (229 mg, 1.083 mmol) was added to the solution. After stirring for 3 h at room temperature, the resulting mixture was diluted with EA and washed with water and brine, dried over MgSO4, and filtered, and the solvent was removed in vacuo. Lithium hydroxide was added to the mixture of methyl N-[(3-{2-[3-cyano-4-(2-methylpropyl)phenyl]-5-pyrimidinyl}-2-ethylphenyl)methyl]-N-methylglycinate (247 mg, 0.541 mmol), Isopropanol (2.5 mL) and Water (2.5 mL). The mixture was stirred at room temperature for 2 hrs. The mixture was neutralized with AcOH and evaporated in vacuo, dissolved in DMF and purified by Mass Directed AutoPrep N-[(3-{2-[3-cyano-4-(2-methylpropyl)phenyl]-5-pyrimidinyl}-2-ethylphenyl)methyl]-N-methylglycine (E21) (174 mg) as a TFA salt.

The following examples were prepared using procedures described for Example 21.

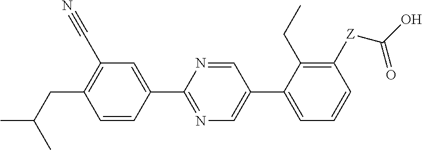

| Example | Z—COOH | characterization |
|---|---|---|
| 21 | 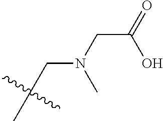 [SMILES: *CN(C)CC(=O)O] | δH (DMSO-d₆, 400 MHz): 0.92 (9 H, m), 2.00 (1 H, dt), 2.76 (7 H, m), 4.10 (2 H, m), 4.39 (2 H, m), 7.43 (2 H, m), 7.65 (2 H, m), 8.68 (2 H, m), 8.96 (2 H, s). δF (DMSO-d₆, 376 MHz): −73.8. MS (ES): C₂₇H₃₀N₄O₂ requires 442.2; found 443.1 (M + H⁺). |
| 22 | 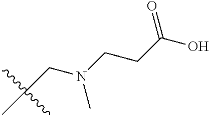 [SMILES: *CN(C)CCC(=O)O] | δH (DMSO-d₆, 400 MHz): 0.92 (9 H, m), 2.00 (1 H, dt), 2.68 (3 H, m), 2.80 (7 H, m), 4.42 (2 H, m), 7.45 (2 H, m), 7.66 (2 H, m), 8.66 (1 H, dd), 8.72 (1 H, d), 8.96 (2 H, s), 9.28 (1 H, br. s.), 12.82 (1 H, br. s.). δF (DMSO-d₆, 376 MHz): −73.6. MS (ES): C₂₈H₃₂N₄O₂ requires 456.2; found 457.3 (M + H⁺). |
| 23 | 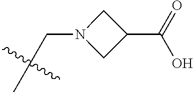 [SMILES: *CN1CC(C(=O)O)C1] | δH (DMSO-d₆, 400 MHz): 0.91 (9 H, m), 2.01 (1 H, dt), 2.65 (2 H, m), 2.78 (2 H, m), 3.66 (1 H, m), 4.30 (4 H, m), 4.57 (2 H, m), 7.42 (2 H, m), 7.52 (1 H, s), 7.67 (1 H, d), 8.66 (1 H, dd), 8.71 (1 H, d), 8.94 (2 H, s), 10.22 (1 H, br. s.), 13.20 (1 H, br. s.). δF (DMSO-d₆, 376 MHz): −73.6. MS (ES): C₂₈H₃₀N₄O₂ requires 454.2; found 455.3 (M + H⁺). |
| 24 | 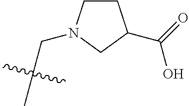 [SMILES: *CN1CCC(C(=O)O)C1] | δH (DMSO-d₆, 400 MHz): 0.94 (9 H, m), 2.00 (3 H, m), 2.33 (1 H, m), 2.53 (1 H, m), 2.69 (6 H, m), 2.94 (1 H, m), 3.67 (2 H, m), 7.20 (1 H, d), 7.30 (1 H, t), 7.45 (1 H, d), 7.66 (1 H, d), 8.66 (1 H, dd), 8.71 (1 H, d), 8.97 (2 H, m). MS (ES): C₂₉H₃₂N₄O₂ requires 468.2; found 469.2 (M + H⁺). |
| 25 | 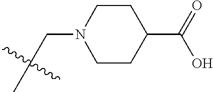 [SMILES: *CN1CCC(C(=O)O)CC1] | δH (DMSO-d₆, 400 MHz): 0.84 (9 H, m), 1.71 (2 H, m), 1.95 (4 H, m), 2.66 (2 H, m), 2.71 (2 H, m), 3.11 (2 H, m), 3.43 (2 H, m), 4.38 (2 H, m), 7.39 (2 H, m), 7.60 (2 H, m), 8.61 (2 H, m), 8.91 (2 H, s), 9.14 (1 H, br. s.), 12.52 (1 H, br. s.). δF (DMSO-d₆, 376 MHz): −73.9. MS (ES): C₃₀H₃₄N₄O₂ requires 482.2; found 483.2 (M + H⁺). |

### EXAMPLE 26

1-{[3-(2-{3-cyano-4-[(1-methylethyl)oxy]phenyl}-5-pyrimidinyl)-2-ethylphenyl]methyl}-3-azetidinecarboxylic acid trifluoroacetate (E26)

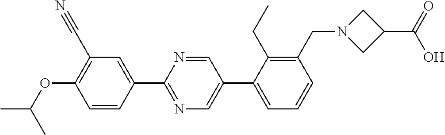 [SMILES: CCc1c(CN2CC(C(=O)O)C2)cccc1-c1cnc(-c2ccc(OC(C)C)c(C#N)c2)nc1]

To a solution of methyl 1-{[3-(2-{3-cyano-4-[(1-methylethyl)oxy]phenyl}-5-pyrimidinyl)-2-ethylphenyl]methyl}-3-azetidinecarboxylate (D59) (315 mg, 0.669 mmol) in Isopropanol (4 mL) and Water (1.000 mL) stirred under nitrogen at room temperature was added NaOH (1.339 mL, 2.68 mmol) in one charge. The reaction mixture was stirred at room temperature for 2 h. Isopropanol was removed in vacuo. The residue was dissolved in water and acidified with 1N HCl aq. solution to pH=5. The samples were dissolved in THF (6 mL) and purified by Mass Directed AutoPrep. The solvent was freeze dried to afford the required product 1-{(3-(2-{3-cyano-4-[(1-methylethyl)oxy]phenyl}-5-pyrimidinyl)-2-ethylphenyl]methyl}-3-azetidinecarboxylic acid (E26) (150 mg) as a TFA salt. δH (DMSO-d₆, 400 MHz): 0.93 (3H, t), 1.37 (6H, d), 2.63 (2H, m), 3.53 (1H, m), 3.93 (2H, m), 4.01 (2H, m), 4.26 (2H, s), 4.92 (1H, m), 7.29 (1H, d), 7.37 (1H, t), 7.46 (2H, m), 8.64 (2H, m), 8.87 (2H, s). δF (DMSO-d₆, 376 MHz): −73.6. MS (ES): C₂₇H₂₈N₄O₃ requires 456.2; found 457.0 (M+H⁺).

### EXAMPLE 27-33

### EXAMPLE 27

N-{2-[3-(2-{3-chloro-4-[(1-methylethyl)oxy]phenyl}-5-pyrimidinyl)-2-ethylphenyl]ethyl}-N-methylglycine (E27)

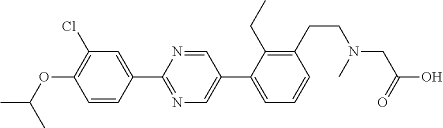 [SMILES: CCc1c(CCN(C)CC(=O)O)cccc1-c1cnc(-c2ccc(OC(C)C)c(Cl)c2)nc1]

To a solution of [3-(2-{3-chloro-4-[(1-methylethyl)oxy]phenyl}-5-pyrimidinyl)-2-ethylphenyl]acetaldehyde (D64) (300 mg, 0.243 mmol), sodium acetate (100 mg, 1.216 mmol) and acetic acid (0.15 mL, 2.62 mmol) in Methanol (10 mL) stirred under nitrogen at room temperature was added methyl N-methylglycinate (170 mg, 1.216 mmol) in one charge. The reaction mixture was stirred for 0.5 h at room temperature. Methanol was evaporated off in vacuo and then the residue was dissolved in Dichloromethane (DCM) (10.00 mL). Sodium triacetoxyborohydride (258 mg, 1.216 mmol) was added to the above mixture. The mixture was stirred overnight at room temperature. The reaction mixture was partitioned between ethyl acetate and water. The organic phase was dried over sodium sulphate and evaporated in vacuoto afford methyl N-{2-[3-(2-{3-chloro-4-[(1-methylethyl)oxy]phenyl}-5-pyrimidinyl)-2-ethylphenyl]ethyl}-N-methylglycinate (117 mg). Then to a solution of methyl N-{2-[3-(2-{3-chloro-4-[(1-methylethyl)oxy]phenyl}-5-pyrimidinyl)-2-ethylphenyl]ethyl}-N-methylglycinate (117 mg, 0.243 mmol) in Isopropanol (3 mL) and Water (0.750 mL) stirred under nitrogen at room temperature was added NaOH (194 mg, 0.971 mmol) in one charge. The reaction mixture was stirred at room temperature for 2 h. Isopropanol was removed in vacuo. The residue was dissolved in water and acidified with 1N HCl eq. solution to pH=5. The samples were dissolved in THF (6 mL) and purified by Mass Directed AutoPrep. The solvent was freeze dried to afford N-{2-[3-(2-{3-chloro-4-[(1-methylethyl)oxy]phenyl}-5-pyrimidinyl)-2-ethylphenyl]ethyl}-N-methylglycine (E27) (10 mg).

The following examples were prepared using procedures described for Example 27.

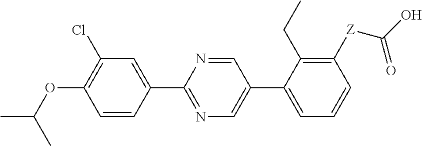

| Example | Z—COOH | characterization |
|---|---|---|
| 27 | 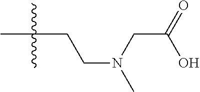 [SMILES: *CCN(C)CC(=O)O] | δH (DMSO-d₆, 400 MHz): 0.96 (3 H, t), 1.35 (6 H, d), 2.56 (4 H, m), 2.92 (3 H, s), 3.35 (4H, m), 4.82 (1 H, m), 7.14 (1 H, dd), 7.26 (3 H, m), 8.37 (1 H, dd), 8.43 (1 H, d), 8.85 (2 H, s). MS (ES): C₂₆H₃₀ClN₃O₃ requires 467.2; found 468.2 (M + H⁺). |
| 28 | 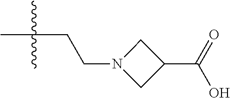 [SMILES: *CCN1CC(C(=O)O)C1] | δH (DMSO-d₆, 400 MHz): 0.94 (3 H, t), 1.35 (6 H, d), 2.54 (2 H, m), 2.65 (2 H, m), 3.17 (3 H, m), 3.42 (4 H, m), 4.82 (1 H, m), 7.11 (1 H, m), 7.24 (1 H, m), 7.30 (1 H, m), 7.35 (1 H, d), 8.36 (1 H, dd), 8.42 (1 H, d), 8.84 (2 H, s). MS (ES): C₂₇H₃₀ClN₃O₃ requires 479.2; found 480.2 (M + H⁺). |
| 29 | 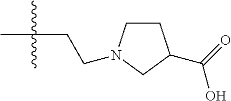 [SMILES: *CCN1CCC(C(=O)O)C1] | δH (DMSO-d₆, 400 MHz): 0.98 (3 H, t), 1.36 (6 H, d), 2.08 (1 H, m), 2.24 (1 H, m), 2.41 (1 H, m), 2.60 (2 H, m), 3.06 (2 H, d), 3.21 (2 H, m), 3.73 (2 H, m), 3.88 (2 H, m), 4.83 (1 H, dt), 7.21 (1 H, m), 7.34 (3 H, m), 8.37 (1 H, dd), 8.43 (1 H, d), 8.85 (2 H, s), 10.00 (1 H, m), 12.98 (1 H, br. s.). δF (DMSO-d₆, 376 MHz): −73.7. MS (ES): C₂₃H₃₂ClN₃O₃ requires 493.2; found 494.0 (M + H⁺). |
| 30 | 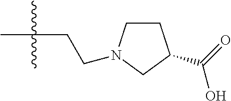 [SMILES: *CCN1CC[C@H](C(=O)O)C1] | δH (DMSO-d₆, 400 MHz): 0.99 (3 H, t), 1.36 (6 H, d), 1.49 (1 H, m), 1.75 (1 H, m), 1.94 (1 H, m), 2.07 (1 H, m), 2.60 (2 H, d), 2.75 (1 H, m), 2.92 (1 H, m), 3.09 (3 H, m), 3.34 (2 H, m), 3.64 (1 H, m), 3.78 (1 H, m), 4.83 (1 H, dt), 7.21 (1 H, m), 7.35 (3 H, m), 8.37 (1 H, dd), 8.43 (1 H, d), 8.85 (2 H, s), 9.45 (1 H, br. s.), 12.94 (1 H, br. s.). MS (ES): C₂₈H₃₂ClN₃O₃ requires 493.2; found 494.2 (M + H⁺). |
| 31 | 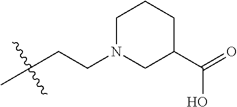 [SMILES: *CCN1CCCC(C(=O)O)C1] | δH (DMSO-d₆, 400 MHz): 0.98 (3 H, t), 1.35 (6 H, d), 1.51 (1 H, m), 1.72 (1 H, m), 1.96 (1 H, m), 2.07 (2 H, m), 2.60 (2 H, m), 2.75 (1 H, m), 2.92 (1 H, m), 3.02 (1 H, m), 3.12 (3 H, d), 3.62 (1 H, m), 3.79 (1 H, m), 4.82 (1 H, dt), 7.21 (1 H, m), 7.35 (3 H, m), 8.37 (1 H, dd), 8.43 (1 H, d), 8.84 (2 H, s), 9.52 (1 H, m). δF (DMSO-d₆, 376 MHz): −73.7. MS (ES): C₂₉H₃₄ClN₃O₃ requires 507.2; found 508.2 (M + H⁺). |
| 32 | 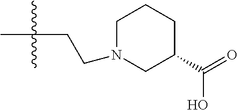 [SMILES: *CCN1CCC[C@H](C(=O)O)C1] | δH (DMSO-d₆, 400 MHz): 0.99 (3 H, t), 1.36 (6 H, d), 1.49 (1 H, d), 1.75 (1 H, m), 1.94 (1 H, m), 2.07 (1 H, m), 2.60 (2 H, m), 2.74 (1 H, m), 2.95 (1 H, m), 3.11 (3 H, m), 3.34 (2 H, m), 3.78 (3 H, m), 3.80 (1 H, m), 4.83 (1 H, dt), 7.21 (1 H, m), 7.36 (3 H, m), 8.37 (1 H, dd), 8.43 (1 H, d), 8.85 (2 H, s), 9.45 (1 H, br. s.), 12.94 (1 H, br. s.). δF (DMSO-d₆, 376 MHz): −73.7. MS (ES): C₂₉H₃₄ClN₃O₃ requires 507.2; found 508.3 (M + H⁺). |
| 33 | 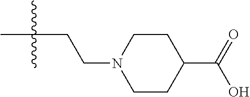 [SMILES: *CCN1CCC(C(=O)O)CC1] | δH (DMSO-d₆, 400 MHz): 0.95 (3 H, t), 1.36 (6 H, m), 1.57 (2 H, m), 1.80 (2 H, m), 2.06 (2 H, m), 2.19 (2 H, m), 2.33 (1 H, m), 2.56 (1 H, m), 2.67 (1 H, m), 2.83 (2 H, m), 2.90 (2 H, m), 4.82 (1 H, dt), 7.11 (1 H, m), 7.25 (1 H, m), 7.33 (2 H, m), 8.36 (1 H, dd), 8.43 (1 H, d), 8.85 (2 H, s). MS (ES): C₂₉H₃₄ClN₃O₃ requires 507.2; found 508.2 (M + H⁺). |

### EXAMPLE 34-40

### EXAMPLE 34

N-[2-(3-{2-[3-cyano-4-(2-methylpropyl)phenyl]-5-pyrimidinyl}-2-ethylphenyl)ethyl]-N-methylglycine trifluoroacetate (E34)

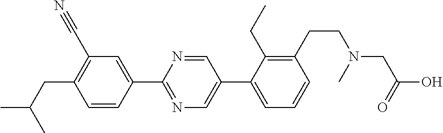 [SMILES: CCc1c(CCN(C)CC(=O)O)cccc1-c1cnc(-c2ccc(CC(C)C)c(C#N)c2)nc1]

The mixture of 5-{5-[2-ethyl-3-(2-oxoethyl)phenyl]-2-pyrimidinyl}-2-(2-methylpropyl)benzonitrile (D66) (100 mg, 0.261 mmol), methyl N-methylglycinate (182 mg, 1.304 mmol), and acetic acid (0.045 mL, 0.782 mmol) in Dichloromethane (DCM) (4 mL) was stirred for 15 min at room temperature. Sodium triacetoxyborohydride (111 mg, 0.522 mmol) was added to the above solution. After stirring 3 h at room temperature, the resulting mixture was diluted with EA and washed with water and brine, dried over MgSO4, and filtered. The solvent was removed in vacuo to afford methyl N-[2-(3-{2-[3-cyano-4-(2-methylpropyl)phenyl]-5-pyrimidinyl}-2-ethylphenyl)ethyl]-N-methylglycinate (123 mg). Lithium hydroxide (14.27 mg, 0.340 mmol) was added to the mixture of methyl N-[2-(3-{2-[3-cyano-4-(2-methylpropyl)phenyl]-5-pyrimidinyl}-2-ethylphenyl)ethyl]-N-methylglycinate (80 mg, 0.170 mmol), Isopropanol (2.5 mL) and Water (2.500 mL). The mixture was stirred at room temperature for 2 hrs. The reaction was neutralized with AcOH and evaporated in vacuo, dissolved in DMF and purified by Mass Directed AutoPrep to afford N-[2-(3-{2-[3-cyano-4-(2-methylpropyl)phenyl]-5-pyrimidinyl}-2-ethylphenyl)ethyl]-N-methylglycine (E34) (20 mg) as a TFA salt.

The following examples were prepared using procedures described for Example 34.

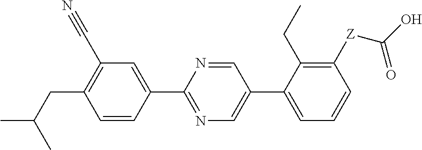

| Example | Z—COOH | characterization |
|---|---|---|
| 34 | 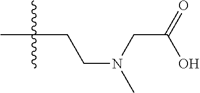 [SMILES: *CCN(C)CC(=O)O] | δH (DMSO-d₆, 400 MHz): 0.96 (9 H, m), 2.00 (1 H, m), 2.63 (2 H, m), 2.78 (2 H, d), 2.95 (3 H, s), 3.11 (4 H, dd), 4.17 (2 H, m), 7.22 (1 H, m), 7.36 (2 H, m), 7.66 (1 H, d), 8.66 (1 H, dd), 8.71 (1 H, d), 8.93 (2 H, s). δF (DMSO-d₆, 376 MHz): −74.7. MS (ES): C₂₈H₃₂N₄O₂ requires 456.3; found 457.3 (M + H⁺). |
| 35 | 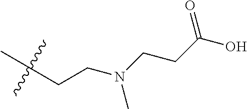 [SMILES: *CCN(C)CCC(=O)O] | δH (DMSO-d₆, 400 MHz): 0.97 (9 H, m), 2.01 (1 H, m), 2.61 (3 H, m), 2.79 (4 H, m), 2.89 (3 H, s), 3.08 (3 H, m), 3.35 (2 H, m), 7.22 (1 H, d), 7.37 (2 H, m), 7.66 (1 H, d), 8.67 (2 H, m), 8.93 (2 H, s). δF (DMSO-d₆, 376 MHz): −73.5. MS (ES): C₂₉H₃₄N₄O₂ requires 470.2; found 471.3 (M + H⁺). |
| 36 | 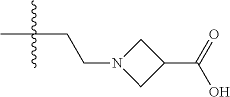 [SMILES: *CCN1CC(C(=O)O)C1] | δH (DMSO-d₆, 400 MHz): 0.95 (9 H, m), 2.00 (1 H, dt), 2.57 (2 H, q), 2.77 (2 H, d), 2.90 (2 H, m), 3.42 (3 H, m), 4.30 (4 H, m), 7.21 (1 H, d), 7.36 (2 H, m), 7.65 (1 H, d), 8.67 (2 H, m), 8.93 (2H, s), 10.36 (1 H, br. s.). δF (DMSO-d₆, 376 MHz): −73.7. MS (ES): C₂₉H₃₂N₄O₂ requires 468.2; found 469.3 (M + H⁺). |
| 37 | 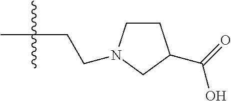 [SMILES: *CCN1CCC(C(=O)O)C1] | δH (DMSO-d₆, 400 MHz): 0.95 (9 H, m), 2.00 (2 H, m), 2.60 (2 H, q), 2.78 (2 H, d), 3.07 (3 H, m), 3.22 (2 H, m), 3.71 (2 H, m), 3.88 (2 H, m), 7.21 (1 H, m), 7.36 (2 H, m), 7.67 (1 H, d), 8.66 (1 H, m), 8.71 (1 H, m), 8.93 (2 H, s). δF (DMSO-d₆, 376 MHz): −73.6. MS (ES): C₃₀H₃₄N₄O₂ requires 482.2; found 483.2 (M + H⁺). |
| 38 | 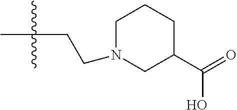 [SMILES: *CCN1CCCC(C(=O)O)C1] | δH (DMSO-d₆, 400 MHz): 0.97 (9 H, m), 1.50 (1 H, m), 1.75 (1 H, m), 2.00 (3 H, m), 2.60 (3 H, m), 2.78 (3 H, m), 2.95 (2 H, m), 3.11 (3 H, m), 3.60 (1 H, m), 3.79 (1 H, m), 7.23 (1 H, m), 7.36 (2 H, m), 7.66 (1 H, d), 8.67 (2 H, m), 8.93 (2 H, s), 9.66 (1 H, br. s.), 12.95 (1 H, br. s.). δF (DMSO-d₆, 376 MHz): −73.6. MS (ES): C₃₁H₃₆N₄O₂ requires 496.3; found 497.3 (M + H⁺). |
| 39 | 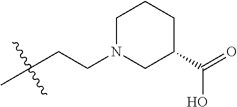 [SMILES: *CCN1CCC[C@H](C(=O)O)C1] | δH (DMSO-d₆, 400 MHz): 0.97 (9 H, m), 1.50 (1 H, m), 1.72 (2 H, m), 2.00 (3 H, tt), 2.62 (2 H, m), 2.78 (4 H, m), 3.10 (4 H, m), 3.61 (1 H, m), 3.76 (1 H, m), 7.22 (1 H, m), 7.36 (2 H, m), 7.66 (1 H, d), 8.67 (2 H, m), 8.93 (2 H, s). δF (DMSO-d₆, 376 MHz): −73.5. MS (ES): C₃₁H₃₆N₄O₂ requires 496.3; found 497.3 (M + H⁺). |
| 40 | 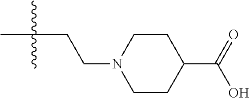 [SMILES: *CCN1CCC(C(=O)O)CC1] | δH (DMSO-d₆, 400 MHz): 0.95 (9 H, m), 1.75 (2 H, m.), 2.02 (4 H, m), 2.59 (3 H, q), 2.77 (2 H, m), 3.06 (5 H, m), 3.54 (2 H, m), 7.21 (1 H, d), 7.35 (2 H, m), 7.66 (1 H, d), 8.67 (2 H, m), 8.93 (2 H, s). δF (DMSO-d₆, 376 MHz): −73.5. MS (ES): C₃₁H₃₆N₄O₂ requires 496.3; found 497.3 (M + H⁺). |

### EXAMPLE 41

4-{[3-(2-{3-chloro-4-[(1-methylethyl)oxy]phenyl}-5-pyrimidinyl)-2-ethylphenyl]oxy}butanoic acid (E41)

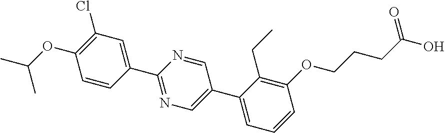 [SMILES: CCc1c(OCCCC(=O)O)cccc1-c1cnc(-c2ccc(OC(C)C)c(Cl)c2)nc1]

To a solution of ethyl 4-{[3-(2-{3-chloro-4-[(1-methylethyl)oxy]phenyl}-5-pyrimidinyl)-2-ethylphenyl]oxy}butanoate (D68) (200 mg, 0.414 mmol) in Isopropanol (3 mL) and Water (1.000 mL) stirred under nitrogen at room temperature was added NaOH (331 mg, 1.656 mmol) in one charge. The reaction mixture was stirred at room temperature for 2 h. Isopropanol was removed in vacuo. The residue was dissolved in water and acidified with 1N HCl aq. solution to pH=6. The samples were dissolved in THF (6 mL) and purified by Mass Directed AutoPrep. The solvent was freeze dried to afford 4-{[3-(2-{3-chloro-4-[(1-methylethyl)oxy]phenyl}-5-pyrimidinyl)-2-ethylphenyl]oxy}butanoic acid (E41) (100 mg). δH (DMSO-d₆, 400 MHz): 1.03 (3H, t), 1.35 (6H, d), 2.00 (2H, m), 2.44 (2H, m), 2.54 (2H, m), 4.06 (2H, t), 4.82 (1H, m), 6.89 (1H, d), 7.07 (1H, d), 7.31 (2H, m), 8.38 (1H, m), 8.42 (1H, m), 8.84 (2H, s), 12.17 (1H, br. s.). MS (ES): C₂₅H₂₇ClN₂O₄ requires 454.2; found 455.0 (M+H⁺).

### EXAMPLE 42

4-{[3-(2-{3-cyano-4-[(1-methylethyl)oxy]phenyl}-5-pyrimidinyl)-2-ethylphenyl]oxy}butanoic acid (E42)

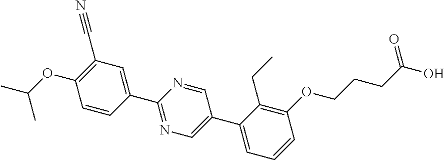 [SMILES: CCc1c(OCCCC(=O)O)cccc1-c1cnc(-c2ccc(OC(C)C)c(C#N)c2)nc1]

Lithium hydroxide (39.0 mg, 0.929 mmol) was added to the mixture of ethyl 4-{[3-(2-{3-cyano-4-[(1-methylethyl)oxy]phenyl}-5-pyrimidinyl)-2-ethylphenyl]oxy}butanoate (D69) (220 mg, 0.465 mmol), Isopropanol (2.5 mL) and Water (2.500 mL). The mixture was stirred at room temperature for 2 hrs. The reaction mixture was neutralized with AcOH and evaporated in vacuo, dissolved in DMF and purified by Mass Directed AutoPrep to afford 4-{[3-(2-{3-cyano-4-[(1-methylethyl)oxy]phenyl}-5-pyrimidinyl)-2-ethylphenyl]oxy}butanoic acid (E42) (46 mg). δH (DMSO-d₆, 400 MHz): 1.03 (3H, t), 1.35 (6H, m), 2.00 (2H, quin), 2.45 (2H, m), 2.53 (2H, m), 4.06 (2H, t), 4.92 (1H, dt), 6.88 (1H, d), 7.07 (1H, d), 7.28 (1H, m), 7.46 (1H, d), 8.65 (2H, m), 8.85 (2H, s), 12.16 (1H, br. s.). δF (DMSO-d₆, 376 MHz): −74.2. MS (ES): C₂₅H₂₇N₃O₄ requires 445.2; found 446.1 (M+H⁺).

### EXAMPLE 43

4-[(3-{2-[3-cyano-4-(2-methylpropyl)phenyl]-5-pyrimidinyl}-2-ethylphenyl)oxy]butanoic acid (E43)

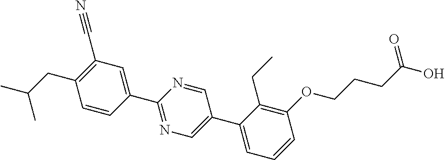 [SMILES: CCc1c(OCCCC(=O)O)cccc1-c1cnc(-c2ccc(CC(C)C)c(C#N)c2)nc1]

Lithium hydroxide (39.2 mg, 0.933 mmol) was added to the mixture of ethyl 4-[(3-{2-[3-cyano-4-(2-methylpropyl)phenyl]-5-pyrimidinyl}-2-ethylphenyl)oxy]butanoate (D70) (220 mg, 0.467 mmol) in Isopropanol (2.5 mL) and Water (2.500 mL). The mixture was stirred at room temperature for 2 hrs. The reaction mixture was neutralized with AcOH and evaporated in vacuo, dissolved in DMF and purified by Mass Directed AutoPrep to afford 4-[(3-{2-[3-cyano-4-(2-methylpropyl)phenyl]-5-pyrimidinyl}-2-ethylphenyl)oxy]butanoic acid (E43) (70 mg). δH (DMSO-d₆, 400 MHz): 0.93 (6H, d), 1.03 (3H, t), 1.99 (3H, do), 2.45 (2H, m), 2.53 (2H, m), 2.75 (2H, d), 4.06 (2H, t), 6.89 (1H, d), 7.08 (1H, d), 7.29 (1H, t), 7.62 (1H, d), 8.65 (2H, m), 8.90 (2H, s), 12.18 (1H, br. s.). δF (DMSO-d₆, 376 MHz): −73.6. MS (ES): C₂₇H₂₉N₃O₃ requires 443.2; found 444.2 (M+H⁺).

### S1P1 Tango Assay

Recombinant EDG1-bla/U2OS cells (contain the human Endothelial Differentiation Gene 1 (EDG1) linked to a TEV protease site and a Gal4-VP16 transcription factor stably integrated into the Tango GPCR-bla U2OS parental cell line) were harvested from growth medium and passaged into assay medium (Invitrogen Freestyle Expression Medium). The cells were starved for 24 hours at 37° C., 5% CO₂, harvested and resuspended in assay medium at a density of ˜200,000 cells/ml. All test compounds were dissolved in DMSO at a concentration of 10 mM and were prepared in 100% DMSO to provide 10 point dose response curves. Test compounds prepared by Bravo (Velocity11) were added to wells in columns 2-11 and 13-22; DMSO was added to wells in columns 12 and 23 as unstimulated controls and assay medium was added to wells in columns 1 and 24 as cell-free controls. An S1P1 agonist was added to wells in row 2, columns 2-11 as stimulated controls and test compounds were added to wells in row 2, columns 13-22 and rows 3-15, columns 2-11/13-22 (row 1 and 16 were empty and not used). Compounds in solution were added to the assay plate (Greiner 781090) using an Echo (Labcyte) dose-response program (50 nl/well). The unstimulated and cell-free controls were loaded with 50 nl/well pure DMSO to ensure that the DMSO concentration was constant across the plate for all assays.

50 μl of the cell suspension was added to each well in columns 2-23 of the plate (˜10,000 cells per well). 50 μl of assay medium was added to each well in the cell-free controls (columns 1 and 24). The cells were incubated overnight at 37° C./5% CO₂. 10 μl of 6× substrate mixture (LiveBLAzer™-FRET B/G substrate (CCF4-AM) Cat #K1096 from Invitrogen, Inc.) was added to each well using Bravo and the plates incubated at room temperature for 2 h in the dark. The plate was finally read on EnVision using one excitation channel (409 nm) and two emission channels (460 nm and 530 nm).

The blue/green emission ratio (460 nm/530 nm) was calculated for each well, by dividing the background-subtracted Blue emission values by the background-subtracted Green emission values. The dose response curve is based on sigmoidal dose-response model. All ratio data was normalized based upon the maximum emission ratio of positive control and minimum emission ratio of negative control (DMSO) on each plate. The intrinsic activity (IA) of each compound would be the normalized percentage of its maximum response after curve fitting.

Exemplified compounds of the invention had a pEC50 ≧6.4. Examples 1-8, 10-11, 13-15, 19-30 and 33-37, 40-43 had a pEC50 ≦7. Examples 1-7, 10, 11, 14-16, 19, 22-28, 30, 33-37 and 40-43 had a pEC50 ≦8. Examples 1, 10, 11, 14-16, 23, 26, 35, 36 and 41-43 had a pEC50 ≦9.

### S1P3 GeneBlazer Assay

GeneBLAzer EDG3-Ga15-NFAT-bla HEK 293T cells (contain the human Endothelial Differentiation G-protein Coupled Receptor 3 (EDG3) and a beta-lactamase reporter gene under control of a NEAT response element and a promiscuous G Protein, Ga15, stably integrated into the GeneBLAzer Ga15-NFAT-bla HEK 293T cell line) were harvested from growth medium and suspended in assay medium (90% DMEM, 10% Charcoal-stripped FBS, 0.1 mM NEAA, 25 mM HEPES (pH 7.3), 100 U/ml penicillin, 100 μg/ml streptomycin) at a density of ˜200,000 cells/ml.

All test compounds were dissolved in DMSO at a concentration of 10 mM and were prepared in 100% DMSO to provide 10 point dose response curves. Test compounds prepared by Bravo (Velocity11) were added to wells in columns 2-11 and 13-22; DMSO was added to wells in columns 12 and 23 as unstimulated controls and assay medium was added to wells in columns 1 and 24 as cell-free controls. An S1P3 agonist was added to wells in row 2, columns 2-11 as stimulated controls and test compounds were added to wells in row 2, columns 13-22 and rows 3-15, columns 2-11/13-22 (row 1 and 16 were empty and not used). Compounds in solution were added to the assay plate (Greiner 781090) using an Echo (Labcyte) dose-response program (50 nl/well). The unstimulated and cell-free controls were loaded with 50 nl/well pure DMSO to ensure that the DMSO concentration was constant across the plate for all assays.

50 μl of the cell suspension was added to each well in columns 2-23 of the plate (˜10,000 cells per well). 50 μl of assay medium was added to each well in the cell-free controls (columns 1 and 24). The cells were incubated overnight at 37° C./5% CO₂.

10 μl of 6× substrate mixture (LiveBLAzer™-FRET B/G substrate (CCF4-AM) Cat # K1096 from Invitrogen, Inc.) was added to each well using Bravo and the plates incubated at room temperature for 2 h in the dark. The plate was finally read on EnVision using one excitation channel (409 nm) and two emission channels (460 nm and 530 nm).

The blue/green emission ratio (460 nm/530 nm) was calculated for each well, by dividing the background-subtracted Blue emission values by the background-subtracted Green emission values. The dose response curve is based on sigmoidal dose-response model. All ratio data was normalized based upon the maximum emission ratio of positive control and minimum emission ratio of negative control (DMSO) on each plate. The intrinsic activity (IA) of each compound would be the normalized percentage of its maximum response after curve fitting.

Exemplified compounds of the invention had a pEC50 <6.3. Exemplified compounds of the invention had a pEC50 <5.7 except Examples 10, 28, 29, 30, 33 and 40. Exemplified compounds of the invention had a pEC50 <5 except Examples 10, 11, 13, 15 and 27-40.

1. A compound of formula (I) or a salt thereof: 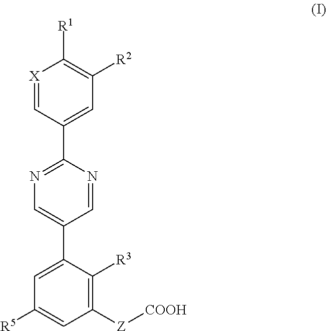
wherein
X is CH or N;
R¹ is C₁₋₆alkoxy or C₁₋₆alkyl;
R² is cyano, CF₃, halogen C₁₋₆alkoxy or CH₂OCH₃;
R³ is C₁₋₆alkoxy or C₁₋₆alkyl;
Z is C₁₋₅alkyl, C₀₋₃alkylOC₁₋₅alkyl or C₀₋₃alkylNR⁴C₀₋₅alkyl, each of which may be optionally substituted by C₁₋₃alkyl;
R⁴ is hydrogen, C₁₋₃alkyl or together with the nitrogen atom to which it is attached forms azetidine, pyrrolidine or piperidine; and
R⁵ is hydrogen, halogen or C₁₋₃alkyl.

2. A compound according to claim 1 selected from:
4-[3-(2-{3-cyano-4-[(1-methylethyl)oxy]phenyl}-5-pyrimidinyl)-2-ethylphenyl]butanoic acid
4-(2-ethyl-3-{2-[4-[(1-methylethyl)oxy]-3-(trifluoromethyl)phenyl]-5-pyrimidinyl}phenyl)butanoic acid
4-(2-ethyl-3-{2-[6-[(1-methylethyl)oxy]-5-(trifluoromethyl)-3-pyridinyl]-5-pyrimidinyl}phenyl)butanoic acid
4-[3-(2-{3-chloro-4-[(1-methylethyl)oxy]phenyl}-5-pyrimidinyl)-2-ethylphenyl]butanoic acid
4-[5-fluoro-3-{2-[4-[(1-methylethyl)oxy]-3-(trifluoromethyl)phenyl]-5-pyrimidinyl}-2-(methyloxy)phenyl]butanoic acid
4-[3-(2-{3-cyano-4-[(1-methylethyl)oxy]phenyl}-5-pyrimidinyl)-5-fluoro-2-(methyloxy)phenyl]butanoic acid
4-[3-(2-{3-cyano-4-[(1-methylethyl)oxy]phenyl}-5-pyrimidinyl)-2-(methyloxy)phenyl]butanoic acid
N-[2-ethyl-3-{2-[4-[(1-methylethyl)oxy]-3-(trifluoromethyl)phenyl]-5-pyrimidinyl}phenyl)methyl]-N-methylglycine
N-ethyl-N-[(2-ethyl-3-{2-[4-[(1-methylethyl)oxy]-3-(trifluoromethyl)phenyl]-5-pyrimidinyl}phenyl)methyl]glycine
N-[2-ethyl-3-{2-[4-[(1-methylethyl)oxy]-3-(trifluoromethyl)phenyl]-5-pyrimidinyl}phenyl)methyl]-b-alanine
1-[(2-ethyl-3-{2-[4-[(1-methylethyl)oxy]-3-(trifluoromethyl)phenyl]-5-pyrimidinyl}phenyl)methyl]-3-azetidinecarboxylic acid
1-{[(2-ethyl-3-{2-[4-[(1-methylethyl)oxy]-3-(trifluoromethyl)phenyl]-5-pyrimidinyl}phenyl)methyl]amino}cyclopropanecarboxylic acid
1-[(2-ethyl-3-{2-[4-[(1-methylethyl)oxy]-3-(trifluoromethyl)phenyl]-5-pyrimidinyl}phenyl)methyl]-4-piperidinecarboxylic acid
4-(3-{2-[3-cyano-4-(2-methylpropyl)phenyl]-5-pyrimidinyl}-2-ethylphenyl)butanoic acid
3-{4-[3-(2-{3-chloro-4-[(1-methylethyl)oxy]phenyl}-5-pyrimidinyl)-2-ethylphenyl]-1-piperidinyl}propanoic acid
4-{4-[3-(2-{3-chloro-4-[(1-methylethyl)oxy]phenyl}-5-pyrimidinyl)-2-ethylphenyl]-1-piperidinyl}butanoic acid
N-{[3-(2-{3-chloro-4-[(1-methylethyl)oxy]phenyl}-5-pyrimidinyl)-2-ethylphenyl]methyl}-N-methylglycine
N-{[3-(2-{3-chloro-4-[(1-methylethyl)oxy]phenyl}-5-pyrimidinyl)-2-ethylphenyl]methyl}-N-methyl-b-alanine
1-{[3-(2-{3-chloro-4-[(1-methylethyl)oxy]phenyl}-5-pyrimidinyl)-2-ethylphenyl]methyl}-3-azetidinecarboxylic acid
1-{[3-(2-{3-chloro-4-[(1-methylethyl)oxy]phenyl}-5-pyrimidinyl)-2-ethylphenyl]methyl}-4-piperidine carboxylic acid
N-[(3-{2-[3-cyano-4-(2-methylpropyl)phenyl]-5-pyrimidinyl}-2-ethylphenyl)methyl]-N-methylglycine
N-[(3-{2-[3-cyano-4-(2-methylpropyl)phenyl]-5-pyrimidinyl}-2-ethylphenyl)methyl]-N-methyl-b-alanine
1-[(3-{2-{3-cyano-4-(2-methylpropyl)phenyl]-5-pyrimidinyl}-2-ethylphenyl)methyl}-3-azetidinecarboxylic acid
1-[(3-{2-[3-cyano-4-(2-methylpropyl)phenyl]-5-pyrimidinyl}-2-ethylphenyl)methyl]-3-pyrrolidinecarboxylic acid
1-[(3-{2-[3-cyano-4-(2-methylpropyl)phenyl]-5-pyrimidinyl}-2-ethylphenyl)methyl]-3-piperidinecarboxylic acid trifluoroacetate
1-{[3-(2-{3-cyano-4-[(1-methylethyl)oxy]phenyl}-5-pyrimidinyl)-2-ethylphenyl]methyl}-3-azetidinecarboxylic acid
N-{2-[3-(2-{3-chloro-4-[(1-methylethyl)oxy]phenyl}-5-pyrimidinyl)-2-ethylphenyl]ethyl}-N-methylglycine
N-{2-[3-(2-{3-chloro-4-[(1-methylethyl)oxy]phenyl}-5-pyrimidinyl)-2-ethylphenyl]ethyl}-3-azetidinecarboxylic acid
1-{2-[3-(2-{3-chloro-4-[(1-methylethyl)oxy]phenyl}-5-pyrimidinyl)-2-ethylphenyl]ethyl}-3-pyrrolidinecarboxylic acid
(3S)-1-{2-[3-(2-{3-chloro-4-[(1-methylethyl)oxy]phenyl}-5-pyrimidinyl)-2-ethylphenyl]ethyl}-3-pyrrolidinecarboxylic acid
1-{2-[3-(2-{3-chloro-4-[(1-methylethyl)oxy]phenyl}-5-pyrimidinyl)-2-ethylphenyl]ethyl}-3-piperidinecarboxylic acid
(3S)-1-{2-[3-(2-{3-chloro-4-[(1-methylethyl)oxy]phenyl}-5-pyrimidinyl)-2-ethylphenyl]ethyl}-3-piperidinecarboxylic acid
1-{2-[3-(2-{3-chloro-4-[(1-methylethyl)oxy]phenyl}-5-pyrimidinyl)-2-ethylphenyl]ethyl}-4-piperidinecarboxylic acid
N-[2-(3-{2-[3-cyano-4-(2-methylpropyl)phenyl]-5-pyrimidinyl}-2-ethylphenyl)ethyl]-N-methylglycine
N-[2-(3-{2-[3-cyano-4-(2-methylpropyl)phenyl]-5-pyrimidinyl}-2-ethylphenyl)ethyl]-N-methyl-b-alanine
1-[2-(3-{2-[3-cyano-4-(2-methylpropyl)phenyl]-5-pyrimidinyl}-2-ethylphenyl)ethyl]-3-azetidinecarboxylic acid
1-[2-(3-{2-[3-cyano-4-(2-methylpropyl)phenyl]-5-pyrimidinyl}-2-ethylphenyl)ethyl]-3-pyrrolidinecarboxylic acid
1-[2-(3-{2-[3-cyano-4-(2-methylpropyl)phenyl]-5-pyrimidinyl}-2-ethylphenyl)ethyl]-3-piperidinecarboxylic acid
(3S)-1-[2-(3-{2-[3-cyano-4-(2-methylpropyl)phenyl]-5-pyrimidinyl}-2-ethylphenyl)ethyl]-3-piperidinecarboxylic acid
1-[2-(3-{2-[3-cyano-4-(2-methylpropyl)phenyl]-5-pyrimidinyl}-2-ethylphenyl)ethyl]-4-piperidinecarboxylic acid
4-{[3-(2-{3-chloro-4-[(1-methylethyl)oxy]phenyl}-5-pyrimidinyl)-2-ethylphenyl]oxy}butanoic acid
4-{[3-(2-{3-cyano-4-[(1-methylethyl)oxy]phenyl}-5-pyrimidinyl)-2-ethylphenyl]oxy}butanoic acid
4-[(3-{2-[3-cyano-4-(2-methylpropyl)phenyl]-5-pyrimidinyl}-2-ethylphenyl)oxy]butanoic acid
and salts thereof.

3. (canceled)

4. (canceled)

5. A process for preparing a pharmaceutical composition comprising mixing a compound according to claim 1 or a pharmaceutically acceptable salt thereof and a pharmaceutically acceptable carrier or excipient.

6. (canceled)

7. A pharmaceutical composition comprising a compound of formula (I) or a pharmaceutically acceptable salt thereof according to claim 1.

8. A method of treatment for conditions or disorders in mammals including humans which can be mediated via the S1P1 receptor, which comprises administering to the sufferer a therapeutically safe and effective amount of a compound of formula (I) or a pharmaceutically acceptable salt thereof according to claim 1.

9. A method of treatment according to claim 8, wherein the condition is multiple sclerosis.
